SEQUENCE LISTING (120 sequences)

SEQ ID NO: 1 - DNA, 25 bp, Artificial sequence
  Primer
gagggaggaagttatggtaggttat

SEQ ID NO: 2 - DNA, 25 bp, Artificial sequence
  Primer
tcctaaccaccttctcaaccttaaa

SEQ ID NO: 3 - DNA, 25 bp, Artificial sequence
  Primer
gagggaggaagttatggtaggttct

SEQ ID NO: 4 - DNA, 25 bp, Artificial sequence
  Primer
gagggaggaagttatggtaggttgt

SEQ ID NO: 5 - DNA, 25 bp, Artificial sequence
  Primer
gagggaggaagttatggtaggtttt

SEQ ID NO: 6 - DNA, 25 bp, Artificial sequence
  Primer
tcctaaccaccttctcaaccttata

SEQ ID NO: 7 - DNA, 25 bp, Artificial sequence
  Primer
tcctaaccaccttctcaaccttaca

SEQ ID NO: 8 - DNA, 25 bp, Artificial sequence
  Primer
tcctaaccaccttctcaaccttaga

SEQ ID NO: 9 - DNA, 21 bp, Artificial sequence
  Probe
agaaggtagtttgaagttggt

SEQ ID NO: 10 - DNA, 18 bp, Artificial sequence
  Primer
ttcgtcgttgtttttcgc

SEQ ID NO: 11 - DNA, 17 bp, Artificial sequence
  Primer
gttaaccgcgaaatccg

SEQ ID NO: 12 - DNA, 15 bp, Artificial sequence
  Probe
aacaacgaatcgcgc

SEQ ID NO: 13 - DNA, 16 bp, Artificial sequence
  Primer
gttggttgttgcggtc

SEQ ID NO: 14 - DNA, 14 bp, Artificial sequence
  Primer
gccaaacccacccg

SEQ ID NO: 15 - DNA, 14 bp, Artificial sequence
  Probe
tccaacacgtccgc

SEQ ID NO: 16 - DNA, 20 bp, Artificial sequence
  Primer
tttcgtttggattcggtaac

SEQ ID NO: 17 - DNA, 15 bp, Artificial sequence
  Primer
cccgaacaaaacgcg

SEQ ID NO: 18 - DNA, 21 bp, Artificial sequence
  Probe
tttattttcgattgagtggat

SEQ ID NO: 19 - DNA, 19 bp, Artificial sequence
  Primer
gtttattcgagtaggacgc

SEQ ID NO: 20 - DNA, 23 bp, Artificial sequence
  Primer
ccgacaacgaaataaaaaaatcg

SEQ ID NO: 21 - DNA, 21 bp, Artificial sequence
  Probe
ttatttagtcggaggtgagga

SEQ ID NO: 22 - DNA, 17 bp, Artificial sequence
  Primer
gggtttaagcggggttc

SEQ ID NO: 23 - DNA, 23 bp, Artificial sequence
  Primer
atttcactctaaaaaatccatcg

SEQ ID NO: 24 - DNA, 14 bp, Artificial sequence
  Probe
tccccgacgactct

SEQ ID NO: 25 - DNA, 18 bp, Artificial sequence
  Primer
gtttcgaggtagtttcgc

SEQ ID NO: 26 - DNA, 22 bp, Artificial sequence
  Primer
ttatatttaccaaacgacaacg

SEQ ID NO: 27 - DNA, 17 bp, Artificial sequence
  Probe
ctcgaaaactcgcgaaa

SEQ ID NO: 28 - DNA, 18 bp, Artificial sequence
  Primer
aaatttaggcggtagtgc

SEQ ID NO: 29 - DNA, 19 bp, Artificial sequence
  Primer
cctattaaaaacacccgcg

SEQ ID NO: 30 - DNA, 16 bp, Artificial sequence
  Probe
ctacgcgccctcacaa

SEQ ID NO: 31 - DNA, 17 bp, Artificial sequence
  Primer
ttttttacgtaggcggc

SEQ ID NO: 32 - DNA, 15 bp, Artificial sequence
  Primer
ccgctaaaaacgccg

SEQ ID NO: 33 - DNA, 14 bp, Artificial sequence
  Probe
cccgtacccgcgcc

SEQ ID NO: 34 - DNA, 21 bp, Artificial sequence
  Primer
ggttttttttagtacgttcgc

SEQ ID NO: 35 - DNA, 23 bp, Artificial sequence
  Primer
cgcaactaaataaaatcatttcg

SEQ ID NO: 36 - DNA, 21 bp, Artificial sequence
  Probe
taaactaactactacgaccgc

SEQ ID NO: 37 - DNA, 15 bp, Artificial sequence
  Primer
ggggttcgagtggtc

SEQ ID NO: 38 - DNA, 15 bp, Artificial sequence
  Primer
cgtcccctaaacgcg

SEQ ID NO: 39 - DNA, 18 bp, Artificial sequence
  Probe
tatacgatcggagcgttt

SEQ ID NO: 40 - DNA, 19 bp, Artificial sequence
  Primer
gtttaggggttttttcggc

SEQ ID NO: 41 - DNA, 17 bp, Artificial sequence
  Primer
ctaactaaaacgccgcg

SEQ ID NO: 42 - DNA, 22 bp, Artificial sequence
  Probe
tagttttgtattgtaggagcgc

SEQ ID NO: 43 - DNA, 15 bp, Artificial sequence
  Primer
gttcgggttttgcgc

SEQ ID NO: 44 - DNA, 18 bp, Artificial sequence
  Primer
ctaaaaactcctccgacg

SEQ ID NO: 45 - DNA, 15 bp, Artificial sequence
  Probe
acgcgaacgcgacgc

SEQ ID NO: 46 - DNA, 21 bp, Artificial sequence
  Primer
cggttttcgttcgttttttcg

SEQ ID NO: 47 - DNA, 20 bp, Artificial sequence
  Primer
gtaacttccgccttctacgc

SEQ ID NO: 48 - DNA, 18 bp, Artificial sequence
  Probe
ctaaaatacccgataaac

SEQ ID NO: 49 - DNA, 24 bp, Artificial sequence
  Primer
tttagtttcggcggttatattttc

SEQ ID NO: 50 - DNA, 22 bp, Artificial sequence
  Primer
ctactctcgaaaaaactcgacg

SEQ ID NO: 51 - DNA, 15 bp, Artificial sequence
  Probe
caccgaatccgcgca

SEQ ID NO: 52 - DNA, 17 bp, Artificial sequence
  Primer
ttagttgcgcgttgatc

SEQ ID NO: 53 - DNA, 14 bp, Artificial sequence
  Primer
ccccgccgaaaacg

SEQ ID NO: 54 - DNA, 13 bp, Artificial sequence
  Probe
tagcgggtcgcgc

SEQ ID NO: 55 - DNA, 19 bp, Artificial sequence
  Primer
ttttgtattgtaggagcgc

SEQ ID NO: 56 - DNA, 12 bp, Artificial sequence
  Primer
acgcccccgacg

SEQ ID NO: 57 - DNA, 15 bp, Artificial sequence
  Probe
tagggttcgggtttc

SEQ ID NO: 58 - DNA, 24 bp, Artificial sequence
  Primer
ggagttatttaagcgtgattgttc

SEQ ID NO: 59 - DNA, 25 bp, Artificial sequence
  Primer
aataaaatacaaaaaacgaatcgcg

SEQ ID NO: 60 - DNA, 17 bp, Artificial sequence
  Probe
aaacttcctcgccgcga

SEQ ID NO: 61 - DNA, 29 bp, Artificial sequence
  Primer
acgttggatggctgcggtaacgcagtgac

SEQ ID NO: 62 - DNA, 29 bp, Artificial sequence
  Primer
acgttggatgcaggagccctcaggcgagt

SEQ ID NO: 63 - DNA, 28 bp, Artificial sequence
  Primer
acgttggatgaagcttcggtcccggtcc

SEQ ID NO: 64 - DNA, 30 bp, Artificial sequence
  Primer
acgttggatgcgcgctgcacttaatagtgg

SEQ ID NO: 65 - DNA, 29 bp, Artificial sequence
  Primer
acgttggatgaccgcgggcgcaatctgag

SEQ ID NO: 66 - DNA, 30 bp, Artificial sequence
  Primer
acgttggatgttacctaagccggcgggact

SEQ ID NO: 67 - DNA, 30 bp, Artificial sequence
  Primer
acgttggatgtatggagggagagttggcta

SEQ ID NO: 68 - DNA, 30 bp, Artificial sequence
  Primer
acgttggatgttttcgtccgagtcttcccc

SEQ ID NO: 69 - DNA, 30 bp, Artificial sequence
  Primer
acgttggatgttagggcccgaaccccagaa

SEQ ID NO: 70 - DNA, 30 bp, Artificial sequence
  Primer
acgttggatgtggagcggagaacggcgttt

SEQ ID NO: 71 - DNA, 30 bp, Artificial sequence
  Primer
acgttggatgagagaaggagaggaaggcag

SEQ ID NO: 72 - DNA, 30 bp, Artificial sequence
  Primer
acgttggatgctcctaatcttcagaaccgc

SEQ ID NO: 73 - DNA, 29 bp, Artificial sequence
  Primer
acgttggatggaggctctgtgctcgcggg

SEQ ID NO: 74 - DNA, 29 bp, Artificial sequence
  Primer
acgttggatgggcgccctataaaacccag

SEQ ID NO: 75 - DNA, 29 bp, Artificial sequence
  Primer
acgttggatgcccgtccgcttaggagact

SEQ ID NO: 76 - DNA, 29 bp, Artificial sequence
  Primer
acgttggatgccaagccaggagctgaggt

SEQ ID NO: 77 - DNA, 28 bp, Artificial sequence
  Primer
acgttggatgtgctcagccgcgctcccc

SEQ ID NO: 78 - DNA, 29 bp, Artificial sequence
  Primer
acgttggatgctcaccccccagcgagcag

SEQ ID NO: 79 - DNA, 29 bp, Artificial sequence
  Primer
acgttggatgagtttccggcgtccggaag

SEQ ID NO: 80 - DNA, 30 bp, Artificial sequence
  Primer
acgttggatgctggaaacggaacactggat

SEQ ID NO: 81 - DNA, 29 bp, Artificial sequence
  Primer
acgttggatgccacagccgctgcggatca

SEQ ID NO: 82 - DNA, 30 bp, Artificial sequence
  Primer
acgttggatgtccaggagagaaagaaagcg

SEQ ID NO: 83 - DNA, 29 bp, Artificial sequence
  Primer
acgttggatgaggaccaggcttgcatggg

SEQ ID NO: 84 - DNA, 29 bp, Artificial sequence
  Primer
acgttggatgatgcggtccggcgtgtcca

SEQ ID NO: 85 - DNA, 29 bp, Artificial sequence
  Primer
acgttggatgaccaagagccccgggccag

SEQ ID NO: 86 - DNA, 29 bp, Artificial sequence
  Primer
acgttggatgtgtcccggaacgtccacag

SEQ ID NO: 87 - DNA, 29 bp, Artificial sequence
  Primer
acgttggatgtctgcaggactgcgggagc

SEQ ID NO: 88 - DNA, 29 bp, Artificial sequence
  Primer
acgttggatgcagcgggctgagattgttg

SEQ ID NO: 89 - DNA, 30 bp, Artificial sequence
  Primer
acgttggatgtcctctaagcgctgggcgat

SEQ ID NO: 90 - DNA, 28 bp, Artificial sequence
  Primer
acgttggatgcgccgccgccccagtgtc

SEQ ID NO: 91 - DNA, 15 bp, Artificial sequence
  Primer
gctgctcttgcgatg

SEQ ID NO: 92 - DNA, 15 bp, Artificial sequence
  Primer
cggcgtggaggaaag

SEQ ID NO: 93 - DNA, 16 bp, Artificial sequence
  Primer
tctgagcccctgccca

SEQ ID NO: 94 - DNA, 16 bp, Artificial sequence
  Primer
ggcggctggtaaccca

SEQ ID NO: 95 - DNA, 17 bp, Artificial sequence
  Primer
ccccagaactcccgagg

SEQ ID NO: 96 - DNA, 17 bp, Artificial sequence
  Primer
ggaaggcagcaatttaa

SEQ ID NO: 97 - DNA, 17 bp, Artificial sequence
  Primer
gggctggggtggcgcgt

SEQ ID NO: 98 - DNA, 18 bp, Artificial sequence
  Primer
gcttagggaactctcctt

SEQ ID NO: 99 - DNA, 19 bp, Artificial sequence
  Primer
agccccctgccctccgcga

SEQ ID NO: 100 - DNA, 19 bp, Artificial sequence
  Primer
gtccaaggaccgagctctt

SEQ ID NO: 101 - DNA, 19 bp, Artificial sequence
  Primer
acgctgcggatcatggtga

SEQ ID NO: 102 - DNA, 20 bp, Artificial sequence
  Primer
ccctccgcccagggtccaaa

SEQ ID NO: 103 - DNA, 20 bp, Artificial sequence
  Primer
ggtcagggccaggaagctgt

SEQ ID NO: 104 - DNA, 21 bp, Artificial sequence
  Primer
cctgccaagccgccctggtga

SEQ ID NO: 105 - DNA, 21 bp, Artificial sequence
  Primer
gggcccggctccgcgcggtcg

SEQ ID NO: 106 - DNA, 76 bp, Artificial sequence
  Competitor
gctgcggtaacgcagtgaccgcgctccaggtccgcgtctcttgcgatggttcccccactcgcctgagggctcctgc

SEQ ID NO: 107 - DNA, 80 bp, Artificial sequence
  Competitor
aagcttcggtcccggtcccccacctcccaccccagagcggcgtggaggaaaggaggagcccactattaagtgcagcgcgc

SEQ ID NO: 108 - DNA, 66 bp, Artificial sequence
  Competitor
accgcgggcgcaatctgagcccctgcccaggcgcagcggcctctcagtcccgccggcttaggtaac

SEQ ID NO: 109 - DNA, 71 bp, Artificial sequence
  Competitor
gttttcgtccgagtcttccccggcagtaggcggtgggttacccgcagccctagccaactctccctccatac

SEQ ID NO: 110 - DNA, 72 bp, Artificial sequence
  Competitor
gttagggcccgaaccccagaactcccgagggagggtgtgtgccgcgcgcgggaaacgccgttctccgctcca

SEQ ID NO: 111 - DNA, 62 bp, Artificial sequence
  Competitor
gagagaaggagaggaaggcagcaatttaagtccctgcggcccgcggttctgaagattaggag

SEQ ID NO: 112 - DNA, 75 bp, Artificial sequence
  Competitor
gaggctctgtgctcgcggggcggacgcggtctcggcggtggtggcgcgtggcgccgctgggttttatagggcgcc

SEQ ID NO: 113 - DNA, 69 bp, Artificial sequence
  Competitor
gcccgtccgcttaggagactctccttggggctttcccggtcggccgcctgacctcagctcctggcttgg

SEQ ID NO: 114 - DNA, 65 bp, Artificial sequence
  Competitor
tgctcagccgcgctccccgccctccgcgagcggttcctcctcccggctgctcgctggggggtgag

SEQ ID NO: 115 - DNA, 60 bp, Artificial sequence
  Competitor
gagtttccggcgtccggaaggaccgagctcttgtcgcggatccagtgttccgtttccagc

SEQ ID NO: 116 - DNA, 78 bp, Artificial sequence
  Competitor
gtccaggagagaaagaaagcgcggcggtgtcggtggcggcgcgcggccccagtcaccatgatccgcagcggctgtggc

SEQ ID NO: 117 - DNA, 81 bp, Artificial sequence
  Competitor
aggaccaggcttgcatggggacgggggtctgcgtccgcgtccagggtccaaaggagggcggtggacacgccggaccgcatc

SEQ ID NO: 118 - DNA, 70 bp, Artificial sequence
  Competitor
accaagagccccgggccaggaagctgtgaggcaggggagggccagcgccagctgtggacgttccgggaca

SEQ ID NO: 119 - DNA, 63 bp, Artificial sequence
  Competitor
gtctgcaggactgcgggagcccagccgccctggtgagagaggacaacaatctcagcccgctgc

SEQ ID NO: 120 - DNA, 61 bp, Artificial sequence
  Competitor
tcctctaagcgctgggcgatcggctccgcgcggtcggagccaggacactggggcggcggcg

(19) United States
(12) Patent Application Publication
(10) Pub. No.: US 2024/0271219 A1
(43) Pub. Date: Aug. 15, 2024

(54) METHOD FOR DETECTING COLORECTAL CANCER DNA METHYLATION AND REAGENT

(71) Applicant: Innovation Biomedical Co., Ltd., Wenzhou, Zhejiang (CN)

(72) Inventors: Chunming Ding, Wenzhou (CN); Shengnan Jin, Wenzhou (CN); Dewen Zhu, Wenzhou (CN); Ju Luan, Wenzhou (CN)

(21) Appl. No.: 17/927,526

(22) PCT Filed: Jul. 14, 2020
(86) PCT No.: PCT/CN2020/101835

§ 371 Date: Nov. 23, 2022

(30) Foreign Application Priority Data
May 25, 2020 (CN) 202010453011.0

(51) Int. Cl. C12Q 1/6886; C12Q 1/6851

(52) U.S. Cl. CPC C12Q 1/6886; C12Q 1/6851; C12Q 2600/118; C12Q 2600/154; C12Q 2600/16

(57) ABSTRACT

The present invention is related to methods and reagents for detecting DNA methylation markers for colorectal cancer. Specifically, the present invention involves methods for diagnosis of presence of colorectal cancer in a subject, prognosis of a colorectal cancer patient after surgery, predicting recurrence for a colorectal cancer patient after surgery, and assessing treatment efficacy for a colorectal cancer patient, through detecting DNA methylation marker(s) in cell-free DNA from a test subject. The present invention also involves the markers, diagnostic kits and reagents for the said methods.

### CROSS-REFERENCE TO RELATED APPLICATIONS

This application is a 35 U.S.C. § 371 national stage filing of International Application No. PCT/CN2020/101835 filed on Jul. 14, 2020, which in turn claims priority to Chinese Application No. 202010453011.0, filed May 25, 2020. The entire contents of each of the foregoing applications are included herein by reference in their entirety.

### TECHNICAL FIELD

The present invention relates to the medical diagnostics field, more specifically it involves the early diagnosis, prognosis and treatment efficacy evaluation of colorectal cancer. Additionally, the present invention also relates to kits and reagents for the aforementioned diagnosis, prognosis and treatment efficacy evaluation.

### BACKGROUND

Worldwide, colorectal cancer (CRC) is among the most common malignancies in the digestive tract. According to the statistics, there are 1.8 million newly diagnosed cases and 860 thousand mortalities [1]. In China, CRC ranks fifth in both incidence and mortality [2]. In recent years, CRC incidence and mortality rates continue to rise [2], while these rates in developed countries such as USA are declining [3]. Such rise in China is worth attention and further analysis. On one hand, life style factors such as high fat diet, smoking and excessive alcohol consumption may increase the risk for CRC. Accordingly, preventive measures may contribute to about 35% in reducing cancer incidence. On the other hand, over 90% of CRC patients are over 50 years old. They tend to ignore early symptoms such as blood in stools and changes in bowel routines, resulting in late diagnosis. Late-stage cancer patients suffer from a low five-year survival rate of less than 15%, far lower than early-stage cancer patients. Data showed that early screening in USA contributes 53% in reducing CRC incidence, while therapy only contributes to 12% [3].

Research shows CRC occurs as the result of a series of genetic and epigenetic changes [4], including functional loss of tumor suppressor genes and activation of oncogenes. These changes are considered as driver mutations since they confer selective growth advantage to the mutated cells and drive cell clonal growth to malignant growth. Typical sporadic CRC may only contain 2-8 drive gene mutations, with other changes such as genomic instability and randomly generated passenger genetic defects. As a result, every CRC patient is unique genetically and epigenetically, which is an important factor to be considered in precision medicine. CRC may be subtyped based on these molecular characteristics, with different subtypes showing different phenotypes and prognosis profiles [5, 6]. Among them, DNA methylation is an epigenetic change that has been studied in depth. DNA methylation is catalyzed by methyltransferase (DNMT) which can transfer the methyl group from SAM to the C-5 position of cytosine in the CpG dinucleotide to produce 5′-methyl cytosine [7]. DNA methylation may suppress the transcription of the corresponding genes (such as tumor suppressor genes), thereby silencing gene expression. Abnormal DNA methylation is frequently observed in tumor development. Compared with genetic changes such as gene mutations, abnormal DNA methylation may occur early, widely and in many different forms in the genome. As such, DNA methylation changes may be used as tumor biomarkers for diagnosis, prognosis and personal therapy [8].

Early detection is the key to improve survival rate and cure rate for CRC as survival rate is correlated with at what stage cancer was diagnosed. Over last few decades, CRC incidence and mortality is on the decline in USA, thanks to the wide adoption of screening at about 60% rate. In China, CRC screening is not yet widely adopted, resulting in a far lower rate in early diagnosis. Current screening methods used in the clinical setting include fecal occult blood test (FOBT), fecal immunochemical test (FIT), colonoscopy, Cologuard and Epi proColon. Each method has pros and cons. FOBT is a non-invasive method. It is easy to perform at low cost. However, it suffers from low sensitivity and may be affected by food and drugs. Colonoscopy is the gold standard as it can detect lesions in the entire large bowel. But it requires good large bowel preparation, is invasive and has risk for bleeding and perforation. As such, patient compliance is low. In many regions it is not widely adopted [9]. This is why developing a non-invasive, sensitive and specific screening method is a hot topic for research.

Surgery and adjuvant therapy is the main therapeutic approach for CRC. However, CRC patients underwent curative therapy may have a recurrence rate of 35% after resection. Most patients (80%) recur within the first two years after surgery [10]. Recurrence and metastasis are often detected at a delayed time. High-risk patients (e.g. stage III CRC patients) may need to receive adjuvant chemotherapy to reduce the risk for relapse and metastasis [11, 12], but not all of these patients may benefit from adjuvant therapy. Clinically, TNM staging (T: tumor; N: node; M: metastasis) is routinely used for minimal residual disease (MRD) evaluation and therapy guidance. However, TNM staging is weakly predictive for some stage II and III patients. After finishing the recommended therapy, follow-up visits are recommended to detect recurrence as early as possible for early intervention. However, in reality many recurrences are detected late [13], with only 10-20% of metachronous metastases are cured [14]. Currently available methods such as CT and endoscopy-guided biopsy [15] for recurrence detection have low sensitivity and additionally may contain radiation or invasive. Carcinoembryonic antigen (CEA) is the only recommended blood biomarker for CRC monitoring [15], but it also suffers from low sensitivity [16]. As such, non-invasive prognosis markers with high sensitivity for MRD detection and accurate prognosis may lead to early intervention, which is critical to improve patient outcome.

Neoadjuvant chemo and radiotherapy prior to surgery is an important component of CRC treatment [17] to improve surgical resectability, maintain anal sphincter function and improve disease free survival. Some patients with locally advanced rectal cancer may benefit from tumor regression and better prognosis after neoadjuvant therapy. However, some patients may not sec tumor regression and they have unnecessarily suffered from adverse reaction and delay in surgery. Currently, there is no definitive markers to predict sensitivity to chemo and radiotherapy. It is difficult to determine whether neoadjuvant therapy has resulted in complete regression, as imaging, colonoscopy, tumor marker analysis and physical examination may be inaccurate. Under the backdrop of individualized medicine, it is important to screen for patients who may benefit from neoadjuvant therapy and assess treatment efficacy.

With the continuous development in molecular biology techniques, liquid biopsy techniques analyzing circulating tumor cells and cell-free nucleic acids in blood is becoming a reality, with its importance being increasingly recognized. In terms of CRC screening, liquid biopsy is non-invasive, simple, economical and high in sensitivity, as compared with colonoscopy and FIT. As such, patient compliance is high. It is thus easy to be widely adopted to improve screening coverage. Compared to the gold standard tissue biopsies, liquid biopsy can overcome tumor heterogeneity to reflect more comprehensive tumor characteristics. It is non-invasive and can offer repeated sampling to monitor tumor dynamic changes and response to treatment in time. Existing blood protein markers are susceptible to environmental factors within the body, less stable, long in half-life and have limited accuracy. Imaging methods are less sensitive and may only detect at a later stage of tumor development. As such, liquid biopsy has good clinical values in tumor screening, diagnosis, prognosis, treatment efficacy prediction and high-risk patient follow-up. Its clinical value is seen to improve patient prognosis and survival by identifying patients for in-time treatment [18, 19, 20]. Cell-free DNA (cfDNA) is present in the peripheral blood from cell lysis. Circulating tumor DNA (ctDNA) is part of cfDNA released from tumor cells into the peripheral blood. CtDNA has short half-time, carries tumor mutations, copy number changes and DNA methylation signals. Several studies have demonstrated that ctDNA may be used for CRC screening, diagnosis, MRD detection, recurrence monitoring and prognosis [21].

Epi proColon is the only FDA approved screening kit based on blood liquid biopsy for CRC. It is based on HeavyMethyl real-time PCR [22, 23], which detects one region in the SEPTIN9 gene and an internal control ACTB in a single qPCR reaction. Firstly, bisulfite conversion is performed with cfDNA extracted from plasma, followed by qPCR by adding primers and probes designed for converted SEPTIN9 and ACTB sequences. The probes for the two sequences were labeled with different fluorescence groups to distinguish from each other. Additionally, a non-extendable oligonucleotide blocker is added in qPCR to bind to the non-methylated sequences of SEPTIN9. The block overlaps with primer binding sites to block the amplification of non-methylated DNA. The interpretation of the results is made from the following three steps: (1) negative and positive control samples are processed and analyzed in parallel to ensure the validity of the test results; (2) signal from the internal control is used for quality control of the DNA template in a single PCR reaction; (3) results from three PCR replicates are used to evaluate the DNA methylation level of the test sample.

There are a few shortcomings to the above method:
1) Only a single target region in the SEPTIN9 gene is tested, resulting in lower sensitivity. In a study with an asymptomatic population the overall sensitivity for CRC was 48.2%, at a specificity of 91.5%. The sensitivity for stage I, II, III and IV patients were 35.0%, 63.0%, 46.0% and 77.4%, respectively [24].
2) False positive or false negative results may occur if the blocker did not specifically bind to unmethylated DNA.
3) Limited clinical utility. The Epi proColon assay was only used for CRC screening or to aid the diagnosis of CRC. There are no known large-scale clinical studies to evaluate the potential utility in MRD detection, prognosis or recurrence detection. A study using SEPTIN9 methylation detection for patients during follow-up showed a sensitivity of 71.4% (15/21) for recurrence detection [25]. Another study was only able to detect 1 out of 4 CRC patients with recurrence based on SEPTIN9 methylation [26], showing serious false negative problem.
4) Detection is qualitative due to low concentration of a single marker, even when fluorescence-based quantitative PCR is used. Test results are interpreted only qualitatively (positive or negative). As a result, it is difficult to assess quantitative changes of ctDNA in plasma, and subsequently difficult to assess dynamic changes of tumor load.

Grail is currently developing blood-based “pan-cancer” early screening methods. They first performed whole genome bisulfite sequencing (WGBS) for a large number of blood and tissue samples to establish a pan-cancer DNA methylation database. A panel of over 100,000 DNA methylation regions were identified by machine learning algorithms. Targeted DNA methylation analysis for the above panel was then used for pan-cancer screening [29]. Using this approach, they analyzed the plasma samples of the participants and demonstrated that their method achieved a specificity of 99.3% (false positive rate ≤1%) and an overall detection rate (sensitivity) of 54.9% ((95% CI: 51.0%-58.8%) for 12 cancer types (all stages included). For CRC, when specificity was set as 99.4%, sensitivity for stage I, II, III, IV patients were about 40-50%, 60-70%, 70% and 80-90%, respectively [29].

The grail method has the following shortcomings:
1) High cost and complex data analysis since it employs WGBS. The cost/benefit ratio is poor for clinical adoption as it does not follow health economics of cancer screening.
2) The method is for pan-cancer screening. The sensitivity for CRC screening is low.

In summary, a method for quantitative analysis of DNA methylation markers for early detection of CRC is still needed.

### DETAILED DESCRIPTION

The present invention discloses methods for detecting multiple DNA methylation markers for CRC detection and screening, evaluation of neoadjuvant radio and chemotherapy efficacy, post-surgery prognosis, MRD detection, dynamic follow-up, early detection of recurrence and metastasis, etc. A total of 4 different DNA methylation panels were designed to target multiple CRC-specific DNA methylation regions. Multiplex quantitative methylation-specific PCR (mqMSP) is the main method for detection. Blood samples from CRC, advanced adenoma, polyps, healthy controls, asymptomatic volunteers, esophageal carcinoma and lung cancer patients were collected to validate the feasibility and utility of the present invention. Additionally, methods based on MALDI-TOF mass spectrometry were developed to simultaneously quantify multiple DNA methylation markers to assist plasma ctDNA quantification.

The present invention took advantage of epigenomics technologies for DNA methylome analysis, literature and databases. A total of 105 samples (30 paired cancer and surrounding normal tissues from CRC patients, 15 non advanced adenoma polyp tissues, 15 advanced adenoma tissues, 15 blood samples from healthy volunteers) were processed for sequencing library construction and bisulfite sequencing. Based on the sequencing results, together with bioinformatics and statistics analysis of data from literature and database search, multiple CRC-specific DNA methylation markers such as ATP8B2, LONRF2, FGF12, CHST10, ELOVL2, HSPA1A were identified.

The present invention uses quantitative real-time PCR for the quantification of the total signal for multiple DNA methylation markers. Primers and probes for quantitative methylation specific PCR are first designed for each DNA methylation marker specific for CRC. PCR reaction conditions are optimized in multiple steps for uniplex assays (analyzing one individual DNA methylation marker) and multiplex assays (analyzing multiple DNA methylation markers) to establish a sensitive and reproducible method capable of quantifying the total signal of multiple DNA methylation markers in a single tube, thereby obtaining the total DNA methylation signal for the multiple markers.

The present invention also uses MALDI-TOF mass spectrometry for nucleic acids analysis to quantify the individual signals of multiple markers. The present invention combines DNA methylation sensitive restriction enzyme digestion, real-competitive technique, single base extension and MALDI-TOF mass spectrometry. PCR and extension primers are designed for the DNA methylation markers. Each reaction can quantify 10-20 markers. Sample DNA is first digested by DNA methylation sensitive restriction enzyme(s), subsequently DNA competitors with known amounts for each marker are added prior to PCR amplification to calculate the amounts of target DNA markers based on the ratios of target DNA markers to their respective competitors. This method can assist the validation of potential biomarkers identified from high-throughput sequencing, or can directly quantify ctDNA in the plasma.

Specifically, the present invention involves the following aspects:

In one aspect, the present invention involves methods for testing an individual for the presence of colorectal cancer, making post-operation prognosis for confirmed CRC patients, predicting post-operation recurrence for CRC patients, or evaluating treatment efficacy for CRC patients. The methods detect the DNA methylation markers in the circulating DNA from an individual to assess the DNA methylation levels of the said markers. If the DNA methylation level is higher than the levels from normal controls, then the tested individual may have CRC, or the tested CRC patient may have poor prognosis, or is more susceptible to recurrence after surgery, or is likely to have poor treatment efficacy. The DNA methylation markers may be one or more markers selected from Table 2, Table 3 and Table 8.

In some embodiments in this regard, the DNA methylation markers are detected by mqMSP, and the DNA methylation markers are:
1) One or more markers selected from MBSF9, MBSF10, MBSF15, MBSR5, MBSR6, MBSR7, MBSR8, MBSR9, MBSR11 and MBSR16;
2) One or more markers selected from MBSF9, MBSF8, MBSR13, MBSR16, NDRG4 and QKI;
3) One or more markers selected from MBSF9, MBSF8, MBSR13, NDRG4, QKI, RD1 and RD2;
4) One or more markers selected from MBSF9, MBSF8, MBSR13, QKI, RD1 and RD2.

Optionally, the aforementioned mqMSP also includes the detection of ACTB gene as an internal control.

In some embodiments in this regard, the aforementioned mqMSP uses primers and probes designed for the aforementioned DNA methylation markers, and primers and probe designed for the internal control ACTB, as listed in Table 4.

In some embodiments in this regard, when the DNA methylation markers are one or more markers selected from MBSF9, MBSF8, MBSR13, QKI, RD1 and RD2, RD2_F primer is not used in mqMSP.

In some embodiments in this regard, the detection of DNA methylation markers is performed by mqMSP where DNA methylation markers may be divided into two or more groups, with each group and the internal control using different fluorescence labels.

In some embodiments in this regard, the DNA methylation markers are divided into two groups. Group 1 consists of MBSF9, MBSR16, MBSF8, MBSR13, NDRG4, NPY and QKI, whereas group 2 consists of MBSF15, MBSR5, MBSR6, MBSR7, MBSR8 and MBSR9, and the internal control gene is ACTB. Primers and probes for group 1 markers are listed in Table 5. Primers and probes for group 2 markers are listed in Table 6. Primers and probe for ACTB are listed in Table 7.

In some embodiments in this regard, MALDI-TOF mass spectrometry is used to analyze the DNA methylation markers and the internal control gene, with PCR primers and extension primers for simultaneously amplify competitor sequences with known amounts for the DNA methylation markers, and uses the signal ratios of the DNA methylation markers and their respective competitors to determine the amounts of the DNA methylation markers. The PCR primers, extension primers and competitors for the DNA methylation markers and the internal control are listed in Table 9, Table 10 and Table 11, respectively.

In some embodiments in this regard, the said sample may be selected from body fluids, blood, serum, plasma, urine, saliva, sweat, sputum, semen, mucus, tear, lymphatic fluid, amniotic fluid, interstitial fluid, pulmonary lavage fluid, cerebrospinal fluid, stool and tissues.

In another aspect, the present invention involves one or more DNA methylation markers selected from Table 2, Table 3 and Table 8, for diagnosing CRC for individuals, or for post-surgery prognosis, predicting recurrence or evaluating treatment efficacy with confirmed CRC patients.

In some embodiments in this regard, the markers are selected from MBSF9, MBSF10, MBSF15, MBSR5, MBSR6, MBSR7, MBSR8, MBSR9, MBSR11, MBSR16, MBSF8, MBSR13, RD1, RD2, NPY, NDRG4, QKI, RRB10, RRB13, RRB14, RRB16, RRB17_1, RRB17_2, RRB20, RRB21_4, RRB26_2, RRB2, RRB30, RRB6_1, RRB6_4 and RRB6_5.

In another aspect, the present invention involves kits for diagnosing CRC for individuals, or for post-surgery prognosis, predicting recurrence or evaluating treatment efficacy with confirmed CRC patients. The kits contain reagents for detecting DNA methylation markers, where the markers are one or more markers selected from Table 2, Table 3 and Table 8.

In some embodiments in this regard, the markers are one or more markers selected from MBSF9, MBSF10, MBSF15, MBSR5, MBSR6, MBSR7, MBSR8, MBSR9, MBSR11, MBSR16, MBSF8, MBSR13, RD1, RD2, NPY, NDRG4 and QKI. Optionally, the kits contain reagents for detecting the internal control gene ACTB.

In some embodiments in this regard, kits for detecting the DNA methylation markers and the internal control gene contain the sequences listed in Table 4.

In some embodiments in this regard, DNA methylation markers are one or more markers selected from RRB10, RRB13, RRB14, RRB16, RRB17_1, RRB17_2, RRB20, RRB21_4, RRB26_2, RRB2, RRB30, RRB6_1, RRB6_4 and RRB6_5. Optionally, the kits contain reagents for detecting the internal control gene ACTB.

In some embodiments in this regard, reagents for detecting the DNA methylation markers and the internal control gene contain the sequences listed in Table 9, Table 10 and Table 11.

In some embodiments in this regard, the DNA methylation markers are:
1) One or more markers selected from MBSF9, MBSF10, MBSF15, MBSR5, MBSR6, MBSR7, MBSR8, MBSR9, MBSR11 and MBSR16;
2) One or more markers selected from MBSF9, MBSF8, MBSR13, MBSR16, NDRG4 and QKI;
3) One or more markers selected from MBSF9, MBSF8, MBSR13, NDRG4, QKI, RD1 and RD2;
4) One or more markers selected from MBSF9, MBSF8, MBSR13, QKI, RD1 and RD2.

In another aspect, the present invention involves polynucleotides selected from SEQ ID NOs:1, 2 and 9-120.

In all aspects of the present invention, the primers, probes and competitor sequences are not limited to the ones listed in the above-mentioned Tables and SEQ ID NOs. Sequences for primers, probes and competitors may be at least 80%, preferably at least 85%, more preferably at least 90%, particularly preferably at least 95%, and further preferably at least 99% identical to the ones listed in the above-mentioned Tables and SEQ ID NOs, while still maintaining their respective desired functions. Preferably, the last 10 nucleotides at the 3′ end of the primers are at least 90%, preferably at least 95%, more preferably at least 99% identical to the ones listed in the above-mentioned Tables and SEQ ID NOs. A skilled person in the art may use routine tools to confirm sequence identity.

In another aspect, the present invention involves utilities for kits for diagnosing CRC for individuals, or for post-surgery prognosis, predicting recurrence or evaluating treatment efficacy with confirmed CRC patients, with one or more DNA methylation markers selected from Table 2. Table 3 and Table 8.

In some embodiments in this regard, diagnosing CRC for individuals, or for post-surgery prognosis, predicting recurrence or evaluating treatment efficacy with confirmed CRC patients are performed by the methods of the present invention as disclosed above.

### FIGURE LEGENDS

FIG. 1. Clustering analysis based on the DNA methylation levels of the candidate genes selected by RRBS. X axis represents the genomic locations of the genes. Y axis represents sample types.

FIG. 2. DNA methylation levels for the candidate genes selected by RRBS. X axis represents candidate marker number, Y axis represents DNA methylation level (Meth %). Unfilled box plots represent normal tissues (N), while grey-filled box plots represent tumor tissues (T).

FIG. 3. Amplification curves for different internal control assays. FAM represents fluorescence signals from DNA methylation markers. VIC represents fluorescence signals from internal controls.

FIG. 4. Evaluation of mqMSP specificity. Bis-CRC represents bisulfite treated tumor tissue DNA. Bis-BC represents bisulfite treated buffy coat DNA. BC represents buffy coat DNA without bisulfite treatment. NTC represents no template blank control.

FIG. 5. Evaluation of mqMSP sensitivity. Lines labeled with values between 0-1% represent fluorescence signals for samples with different methylation levels. VIC represents fluorescence signals from internal controls.

FIG. 6. Sensitivity comparisons between uniplex detection of a single DNA methylation marker and multiplex detection of DNA methylation markers.

FIG. 7. Amplification curves for non-specific signal detection for V2 assay development. Target 1 represents fluorescence signals from DNA methylation markers. Target 2 represents fluorescence signals from internal controls.

FIG. 8. Amplification curves for multiple DNA methylation markers using dual-fluorescence assays. Target 1 represents fluorescence signals from DNA methylation markers. Target 2 represents fluorescence signals from internal controls.

FIG. 9. Amplification curves for multiple DNA methylation markers using triple-fluorescence assays. Target 1 and 2 represent fluorescence signals from DNA methylation markers. Target 3 represents fluorescence signals from internal controls. (original FIG. 9 has a typo—there are two Target 1, but no Target 2)

FIG. 10. Quantitation of multiple DNA methylation biomarkers by MALDI-TOF mass spectrometry. Top panel: mass spectra for DNA methylation marker RRB14 in different samples. Bottom panel: mass spectra for DNA methylation marker RRB17_1 in different samples. E-T1, E-N1 and E-B1 represent tumor tissue DNA after enzymatic digestion, normal tissue DNA after enzymatic digestion and buffy coat DNA after enzymatic digestion, respectively. M-T1, M-N1 and M-B1 represent tumor tissue DNA, normal tissue DNA and buffy coat DNA without enzymatic digestion. Triangles mark the peaks for extension primers. Arrows mark the peaks for extension products from the competitors, with neighboring peaks represent extension products from the DNA samples.

FIG. 11. Representative results for detecting DNA methylation in plasma for a positive sample and a negative sample. FAM represents fluorescence signals from DNA methylation markers. VIC represents fluorescence signals from internal controls.

FIG. 12. Plasma DNA methylation levels and changing patterns as detected by V1 mqMSP in different groups. CRC: colorectal cancer; AA: advanced adenoma; Normal: normal controls. Volunteers: self-proclaimed healthy individuals. Y axis represents DNA methylation levels.

FIG. 13: Plasma DNA methylation levels and changing patterns as detected by V1 mqMSP in different stages of CRC. X axis represents different stages of CRC. Y axis represents DNA methylation levels.

FIG. 14. ROC curve for the results from V1 mqMSP assay.

FIG. 15. Plasma DNA methylation levels and changing patterns as detected by V2 mqMSP in different groups. CRC: colorectal cancer; AA: advanced adenoma; Normal: normal controls. Volunteers: self-proclaimed healthy individuals. Y axis represents DNA methylation levels.

FIG. 16. Plasma DNA methylation levels and changing patterns as detected by V2 mqMSP in different stages of CRC.

FIG. 17. ROC curve for the results from V2 mqMSP assay.

FIG. 18. Plasma DNA methylation levels and changing patterns as detected by V4 mqMSP in different groups. CRC: colorectal cancer; AA: advanced adenoma; GI: gastrointestinal inflammation; ESCC: esophageal squamous cell carcinoma; LC: lung cancer. Y axis represents DNA methylation levels.

FIG. 19. Plasma DNA methylation levels and changing patterns as detected by V4 mqMSP in different stages of CRC. X axis represents different stages of CRC. Y axis represents DNA methylation levels.

FIG. 20. ROC curve for the results from V4 mqMSP assay.

FIG. 21. RFS survival curve for 77 CRC patients with their post-surgery ctDNA analyzed by V1 mqMSP.

FIG. 22. Comparison of ctDNA methylation levels before and after surgery for patients with (A) or without (B) recurrence.

FIG. 23. RFS survival curve for CRC patients with recurrence with their post-surgery ctDNA analyzed by V1 mqMSP.

FIG. 24. Correlation between RFS and post-surgery ctDNA methylation levels for patients with recurrence.

FIG. 25. RFS survival curve for CRC patients with follow-up blood samples tested by V1 mqMSP.

FIG. 26. Dynamic changes in plasma ctDNA methylation levels for a patient with neoadjuvant therapy at different time points of treatment and follow-up.

### EXAMPLES

The present invention discovered and validated multiple CRC-specific DNA methylation markers for detection in clinical samples.

The present invention established a multiple detection method (mqMSP) for specific markers. The mqMSP method detects the total DNA methylation levels of multiple DNA methylation markers, with algorithms for data analysis. Such multiple DNA methylation markers may be grouped in many different ways. The biomarkers are chosen to ensure the combinations having good specificity and sensitivity in detection ctDNA based on certain principles including: no or very low background signal in buffy coat; significantly higher methylation levels in tumor tissues than in the surrounding normal tissues; different markers complement each other in different samples; no or very low non-specific signal derived from interference from detecting different markers.

The present invention also established a mqMSP method using three or more fluorescence channels, with one fluorescence for detecting an internal control, and other fluorescence channels for detecting two or more groups of the methylation markers. Algorithms for combining the different fluorescence signals may be used for dynamically monitoring the quantitative changes of ctDNA.

The present invention also established a method based on MALDI-TOF mass spectrometry for multiplex detection capable of simultaneously quantifying individual signals of multiple DNA methylation markers for ctDNA analysis.

Clinical utility 1: Established multiple mqMSP assays with different combinations of DNA methylation markers for CRC screening (4 different assays). Biomarkers include different combinations of multiple regions from SEPTIN9, NDRG4 and QKI genes. A sample tested positive suggests the individual may have colorectal cancer.

Clinical utility 2: Established mqMSP method for prognosis of CRC patients after surgery. Biomarkers include different combinations of multiple regions from SEPTIN9, NDRG4 and QKI genes. A sample tested positive suggests the CRC patient may have poor prognosis.

Clinical utility 3: Established mqMSP methods for post-surgery monitoring and recurrence prediction for CRC patients. Biomarkers include different combinations of multiple regions from SEPTIN9, NDRG4 and QKI genes. A sample tested positive suggests the CRC patients may suffer from recurrence.

Clinical utility 4: Established mqMSP methods for dynamic monitoring of the entire life cycle of CRC management and treatment including assessing neoadjuvant treatment efficacy, post-surgery evaluation, and post-surgery monitoring. Biomarkers include different combinations of multiple regions from SEPTIN9, NDRG4 and QKI genes. A sample tested positive suggests the patients may have unfavorable neoadjuvant treatment outcome and may need further comprehensive evaluation.

The present invention discovered and validated multiple tumor-specific DNA methylation markers including SEPTIN9, NDRG4, QKI, ATP8B2, LONRF2, FGF12, etc. Some biomarkers such as ATP8B2 and HSPA1A have not been reported in the literature for CRC detection. These biomarkers may be further exploited for clinical diagnosis and treatment of CRC patients.

The present invention took advantages of multiplex detection of several DNA methylation markers. Compared with single marker detection methods such as Epi proColon, the methods in the present invention improved detection sensitivity, detected a higher percentages of early CRC patients, reduced false negative detections for early screening. In multiple cohorts with CRC, advanced adenoma, polyps and normal control samples, the current methods achieved better detection than Epi proColon, with a sensitivity of 42-74.4% for stage I CRC and 74.1-84.2% for stage II CRC.

The biomarkers and detection methods of the present invention expanded the clinical utilities as they can be used for CRC screening, CRC patient prognosis, post-surgery monitoring, recurrence detection, and neoadjuvant therapy efficacy evaluation. In a cohort of 86 CRC patients with follow-up, data showed pre-surgery detection rate of 89.5% (stage I: 80%, II: 90%, III: 90.9% IV 85.7%). For 20 patients that recurred, post-surgery ctDNA detections were positive in 11 patients, while post-surgery CEA detections were positive in only 4 patients. Patients with positive post-surgery ctDNA had significantly shorter recurrence free survival (P=0.008). Taken together, these data demonstrated that the present methods have better sensitivity than CEA and have clinical value in CRC prognosis and recurrence monitoring. Additionally, using the current methods, a CRC patient undergone neoadjuvant therapy was monitored in the entire life-cycle of treatment and follow-up. The detection of ctDNA was positive prior to neoadjuvant therapy, changed to negative after neoadjuvant therapy, and remained negative before and after surgery and in all subsequent follow-up time points, suggesting good prognosis. Imaging examinations also showed no evidence of recurrence. This suggests the current methods have clinical value in assessing neoadjuvant therapy efficacy.

The test cost using the methods of the present invention is reasonable at about CNY 80.

The present invention used MALDI-TOF mass spectrometry and further combined with methylation sensitive restriction enzymes, real-competitive PCR, for quantitation of multiple DNA methylation markers such as ATP8B2, LONRF2, FGF12, CHST10, ELOVL2, HSPA1A. The current method can achieve simultaneous and individual quantification of 10-20 biomarkers in a single reaction system to evaluate the methylation changes for both validation of tumor biomarkers in actual research and ctDNA detection in clinical samples.

Additionally, the primers and probes designed for quantitative real-time PCR in the present invention may also be used in digital droplet PCR platforms. The biomarkers in the present invention may also be used for detections in other digestive tract cancers such as esophageal cancer and stomach cancer.

### Example 1

### Screening for CRC-Specific DNA Methylation Markers

### 1. Methods

(1) Screening for CRC-specific DNA methylation markers using RRBS, a high-throughput DNA methylation analysis method, with the following samples: a total of 60 samples (paired tumor and surrounding normal tissues from 15 stage I, 15 stage II CRC patients), 15 benign polyps (non-advanced adenoma), 15 advanced adenoma and blood samples from 15 healthy volunteers.
(2) Extract genomic DNA from the aforementioned 105 samples, quantify DNA by Qubit, and analyze DNA integrity with agarose gel electrophoresis.
(3) DNA library preparation and sequencing: enrich fragments with CpG sites by MspI digestion of the genomic DNA; end repair and purification; adding A tail and purification; adaptor ligation and ligation product purification; recover DNA fragments with size range between 190 to 320 bp by agarose gel electrophoresis and size selection; bisulfite conversion of the recovered DNA; PCR amplification and purification. One μL of each DNA library was analyzed by Agilent Bioanalyzer 2100 to assess the insert size ranges. The libraries with the expected size ranges were quantified by real-time quantitative PCR. DNA libraries passing these quality control steps were sequenced at high-depth by the Illumina Hiseq X Ten sequence analyzer.
(4) Sequencing data analysis

### A. Initial Screening

1) CpGs satisfying depth at least 10 in at least 5 buffy coat DNA, 10 normal tissue DNA, 5 benign polyps DNA, 5 advanced adenoma DNA and 10 tumor tissue DNA; A total of 2,792,068 CpG sites were identified.
2) Wilcoxon rank sum test was performed for each CpG from the last step, CpG sites with p≤0.05 were selected;
3) Screening for CpGs with average methylation in buffy coat (Avg(BC))≤2%, average methylation in normal tissues (Avg(N))≤10%, and methylation difference between tumor and normal tissues calculated by Avg(T)−Avg(N)≥15%; further requiring distance between the two neighboring CpGs satisfying the conditions above to be no more than 150 bp; still further requiring a differentially methylation region (DMR) to contain at least 3 CpGs satisfying all conditions above. A total of 1666 DMRs were identified.

The above DMRs were further compared with the TCGA DNA methylation database (data generated by the Illumina 450K DNA methylation array) containing data from 33 different tumor types to remove DMRs not present in the TCGA database. As result, 614 DMRs remained. For these 614 DMRs, 500 bp upstream and 500 bp downstream sequences were also included, resulting in 553 DMRs.

### B. Further Screening

Additional requirements:
1) RRBS data: Avg(T)−Avg(N)≥15% where Avg(T) and Avg(N) represent average methylation in tumor and normal tissues, respectively.
2) TCGA database: READ_N≤15%; COAD_N≤15%; LIHC_N≤10%; LIHC_T≤10%; STAD_N≤15%; ESCA_N≤15% where READ_N, COAD_N, LIHC_N, LIHC_T, STAD_N and ESCA_N represent the average methylation levels for normal rectum tissues, normal colon tissues, normal liver tissues, liver hepatocellular carcinoma tissues, normal stomach tissues and normal esophageal tissues.

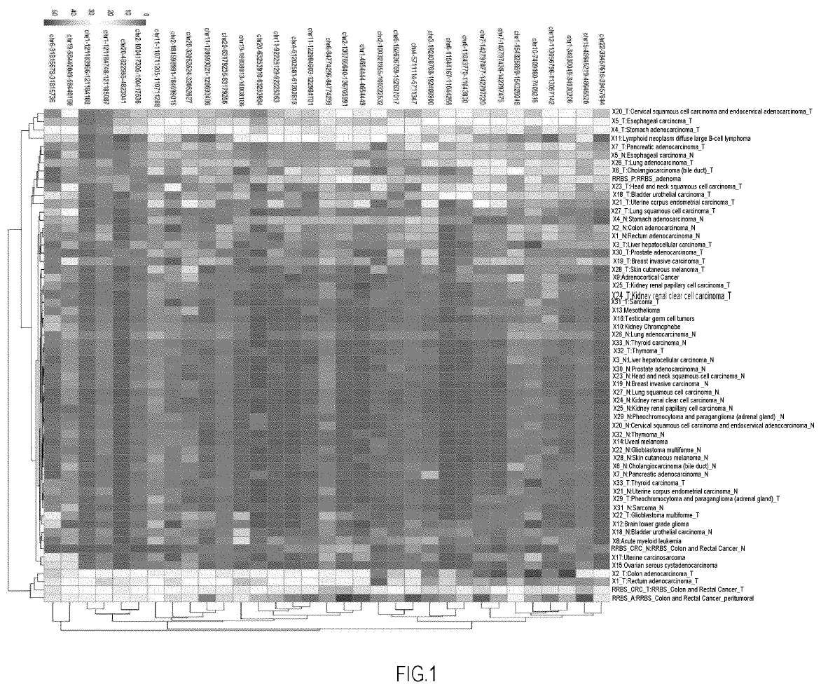

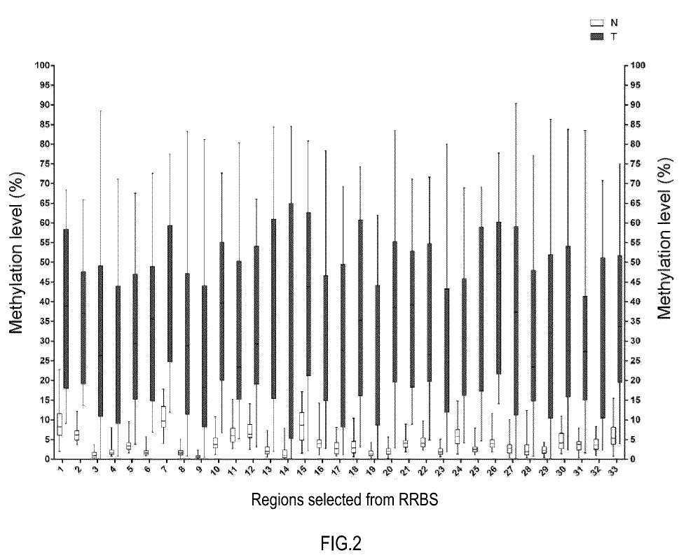

A total of 33 candidate DNA methylation regions (markers) were identified (FIGS. 1 and 2).

### C. The Candidate Regions are Summarized in Table 1.

TABLE 1
Candidate DNA methylation regions

| | | | | | Methylation |
|---|---|---|---|---|---|
| | | | | | difference |
| | | | | | between |
| | | | AUC for CRC | AUC for CRC | CRC and |
| | | | VS normal | VS controls | normal |
| ID | Genomic location | Gene | tissues (RRBS) | (TCGA) | tisues (RRBS) |
| 1 | Chr1: 4654444-4654449 | / | 0.9517 (0.9044, 0.9990) | 0.9622 (0.9458, 0.9786) | 28.48% |
| 2 | Chr1: 34930049-34930206 | | 1.000 (1.000, 1.000) | 0.9587 (0.9409, 0.9766) | 27.39% |
| 3 | Chr1: 121183956-121184188 | FAM72B | 0.8700 (0.7637, 0.9763) | 0.8816 (0.8509, 0.9123) | 27.35% |
| 4 | Chr1: 121184748-121185087 | FAM72B | 0.8656 (0.7614, 0.9697) | 0.8633 (0.8301, 0.8964) | 26.70% |
| 5 | Chr1: 154325859-154326048 | ATP8B2 | 0.9800 (0.9501, 1.010) | 0.9520 (0.9319, 0.9720) | 26.38% |
| 6 | Chr2: 100321955-100322532 | LONRF2 | 1.000 (1.000, 1.000) | 0.9943 (0.9886, 1.000) | 31.72% |
| 7 | Chr2: 136765640-136765991 | THSD7B | 0.9633 (0.9227, 1.004) | 0.9772 (0.9649, 0.9895) | 31.49% |
| 8 | Chr2: 184598991-184599015 | AC096667.1/ | 0.9633 (0.9023, 1.024) | 0.9203 (0.8953, 0.9453) | 28.25% |
| | | ZNF804A | | | |
| 9 | Chr2: 100417305-100417536 | CHST10 | 0.8994 (0.8160, 0.9829) | 0.8617 (0.8251, 0.8983) | 25.41% |
| 10 | Chr3: 192408768-192408900 | FGF12 | 0.9911 (0.9757, 1.007) | 0.9832 (0.9732, 0.9933) | 34.10% |
| 11 | Chr4: 61202581-61202618 | ADGRL3 | 0.9567 (0.9078, 1.006) | 0.9124 (0.8849, 0.9399) | 27.05% |
| 12 | Chr4: 5711114-5711347 | EVC | 0.9222 (0.8386, 1.006) | 0.9557 (0.9372, 0.9742) | 25.51% |
| 13 | Chr6: 11044167-11044255 | ELOVL2 | 0.9589 (0.9116, 1.006) | 0.9108 (0.8831, 0.9386) | 34.49% |
| 14 | Chr6: 31815678-31815736 | HSPA1A | 0.8483 (0.7368, 0.9598) | 0.7598 (0.7185, 0.8010) | 33.74% |
| 15 | Chr6: 84774296-84774299 | / | 0.8756 (0.7664, 0.9847) | 0.9246 (0.9005, 0.9488) | 32.74% |
| 16 | Chr6: 11043770-11043830 | ELOVL2/ | 0.9622 (0.9063, 1.018) | 0.9163 (0.8908, 0.9419) | 28.07% |
| | | ELOVL2-AS1 | | | |
| 17 | Chr6: 152636769-152637017 | SYNE1 | 0.9069 (0.8154, 0.9984) | 0.9502 (0.9309, 0.9695) | 25.95% |
| 18 | Chr7: 142797077-142797220 | TRBJ2-5 | 0.9833 (0.9520, 1.015) | 0.9847 (0.9748, 0.9946) | 35.14% |
| 19 | Chr7: 142797438-142797475 | TRBJ2-7 | 0.9389 (0.8604, 1.017) | 0.9890 (0.9812, 0.9968) | 27.37% |
| 20 | Chr10: 7409160-7409216 | SFMBT2 | 0.9900 (0.9732, 1.007) | 0.9710 (0.9569, 0.9852) | 35.04% |
| 21 | Chr11: 128693021-128693486 | FLI1/ | 0.9989 (0.9952, 1.003) | 0.9719 (0.9578, 0.9861) | 28.96% |
| | | SENCR | | | |
| 22 | Chr11: 92225129-92225363 | / | 0.9789 (0.9490, 1.009) | 0.9552 (0.9368, 0.9736) | 28.61% |
| 23 | Chr11: 110711205-110711288 | ARHGAP20 | 0.9678 (0.9235, 1.012) | 0.9196 (0.8946, 0.9445) | 27.45% |
| 24 | Chr11: 122984603-122984701 | / | 0.9478 (0.8887, 1.007) | 0.9107 (0.8844, 0.9369) | 26.90% |
| 25 | Chr12: 113056796-113057142 | DTX1 | 0.9989 (0.9952, 1.003) | 0.9405 (0.9191, 0.9619) | 33.45% |
| 26 | Chr15: 48645219-48645520 | FBN1 | 1.000 (1.000, 1.000) | 0.9558 (0.9373, 0.9744) | 38.44% |
| 27 | Chr19: 18008013-18008166 | ARRDC2 | 0.8609 (0.7428, 0.9790) | 0.8886 (0.8592, 0.9180) | 31.02% |
| 28 | Chr19: 58440049-58440160 | ZNF132 | 0.9644 (0.9070, 1.022) | 0.9553 (0.9371, 0.9735) | 26.67% |
| 29 | Chr20: 4822955-4823041 | RASSF2 | 0.9111 (0.8177, 1.004) | 0.8963 (0.8678, 0.9247) | 29.38% |
| 30 | Chr20: 63179235-63179266 | AL096828.1 | 0.9656 (0.9088, 1.022) | 0.9139 (0.8881, 0.9397) | 28.51% |
| 31 | Chr20: 32052524-32052627 | HCK | 0.9356 (0.8606, 1.011) | 0.9139 (0.8874, 0.9403) | 26.81% |
| 32 | Chr20: 63253910-63253984 | NKAIN4 | 0.9189 (0.8396, 0.9982) | 0.9187 (0.8935, 0.9439) | 26.05% |
| 33 | Chr22: 39457615-39457644 | MGAT3 | 0.9549 (0.8915, 1.018) | 0.9525 (0.9330, 0.9719) | 26.16% |

### 2. Results

Based on the sequencing data as described above, together with literature data [30-33] and relevant databases, multiple CRC-specific DNA methylation markers such as ATP8B2, LONRF2, FGF12, CHST10, ELOVL2, HSPA1A were discovered, the methylation levels of these markers are significantly higher in CRC than in other sample types (FIGS. 1 and 2).

The methylation markers are listed in Table 2.

TABLE 2
Genes and genomic locations for the DNA methylation
markers discovered in the present invention

| Corresponding gene | Chromosome location |
|---|---|
| SEPTIN9 | Chr17: 77373100-77374054 |
| NDRG4 | Chr16: 58463491-58463554 |
| QKI | Chr6: 163415625-163415707 |
| NPY | Chr7: 24284105-24284197 |
| / | Chr1: 4654444-4654449 |
| / | Chr1: 34930049-34930206 |
| FAM72B | Chr1: 121183956-121184188 |
| FAM72B | Chr1: 121184748-121185087 |
| ATP8B2 | Chr1: 154325859-154326048 |
| LONRF2 | Chr2: 100321955-100322532 |
| THSD7B | Chr2: 136765640-136765991 |
| AC096667.1/ZNF804A | Chr2: 184598991-184599015 |
| CHST10 | Chr2: 100417305-100417536 |
| FGF12 | Chr3: 192408768-192408900 |
| ADGRL3 | Chr4: 61202581-61202618 |
| EVC | Chr4: 5711114-5711347 |
| ELOVL2 | Chr6: 11044167-11044255 |
| HSPA1A | Chr6: 31815678-31815736 |
| / | Chr6: 84774296-84774299 |
| ELOVL2/ELOVL2-AS1 | Chr6: 11043770-11043830 |
| SYNE1 | Chr6: 152636769-152637017 |
| TRBJ2-5 | Chr7: 142797077-142797220 |
| TRBJ2-7 | Chr7: 142797438-142797475 |
| SFMBT2 | Chr10: 7409160-7409216 |
| FLI1/SENCR | Chr11: 128693021-128693486 |
| / | Chr11: 92225129-92225363 |
| ARHGAP20 | Chr11: 110711205-110711288 |
| / | Chr11: 122984603-122984701 |
| DTX1 | Chr12: 113056796-113057142 |
| FBN1 | Chr15: 48645219-48645520 |
| ARRDC2 | Chr19: 18008013-18008166 |
| ZNF132 | Chr19: 58440049-58440160 |
| RASSF2 | Chr20: 4822955-4823041 |
| AL096828.1 | Chr20: 63179235-63179266 |
| HCK | Chr20: 32052524-32052627 |
| NKAIN4 | Chr20: 63253910-63253984 |
| MGAT3 | Chr22: 39457615-39457644 |

### Example 2

### Design and Optimization of Internal Control Assays

An internal control assay targeting the ACTB gene was used as quality control to reflect the sample DNA input amount by coamplifying the internal control gene and the multiple DNA methylation markers in the mqMSP assays. The present invention creatively introduced an artificial mutation in the PCR primers for the internal control gene to reduce the fluorescence signal (VIC) for the internal control assay with the mqMSP reactions to reduce potential inhibition against the signals (FAM) for the DNA methylation markers. The experimental were performed as the following:

### 1. Methods

(1) Seven different primer combinations with primers with or without artificial mutations were designed against the ACTB gene region (chr7:5536826-5536901 (hg38)). The fluorescence probe is labelled by VIC fluorescence.

The seven primer combinations are listed in the following table, with the mutant bases underlined.

| | Forward | Sequence | Reverse | Sequence |
|---|---|---|---|---|
| Combination | primer | (5′ to 3′) | primer | (5′ to 3′) |
| 1 | Fₘᵤₜ₁ | GAGGGAGGAAGTTA | R | TCCTAACCACCTTC |
| | | TGGTAGGTTAT | | TCAACCTTAAA |
| | | (SEQ ID NO: 1) | | (SEQ ID NO: 2) |
| 2 | Fₘᵤₜ₂ | GAGGGAGGAAGTTA | R | TCCTAACCACCTTC |
| | | TGGTAGGTTCT | | TCAACCTTAAA |
| | | (SEQ ID NO: 3) | | (SEQ ID NO: 2) |
| 3 | Fₘᵤₜ₃ | GAGGGAGGAAGTTA | R | TCCTAACCACCTTC |
| | | TGGTAGGTTGT | | TCAACCTTAAA |
| | | (SEQ ID NO: 4) | | (SEQ ID NO: 2) |
| 4 | F | GAGGGAGGAAGTTA | Rₘᵤₜ₁ | TCCTAACCACCTTC |
| | | TGGTAGGTTTT | | TCAACCTTATA |
| | | (SEQ ID NO: 5) | | (SEQ ID NO: 6) |
| 5 | F | GAGGGAGGAAGTTA | Rₘᵤₜ₂ | TCCTAACCACCTTC |
| | | TGGTAGGTTTT | | TCAACCTTACA |
| | | (SEQ ID NO: 5) | | (SEQ ID NO: 7) |
| 6 | F | GAGGGAGGAAGTTA | Rₘᵤₜ₃ | TCCTAACCACCTTC |
| | | TGGTAGGTTTT | | TCAACCTTAGA |
| | | (SEQ ID NO: 5) | | (SEQ ID NO: 8) |
| 7 | F | GAGGGAGGAAGTTA | R | TCCTAACCACCTTC |
| | | TGGTAGGTTTT | | TCAACCTTAAA |
| | | (SEQ ID NO: 5) | | (SEQ ID NO: 2) |

The probe name and sequence are the following:

| Name | Sequence (5′ to 3′) |
|---|---|
| ACTB_probe | 5′VIC-AGAAGGTAGTTTGAAGTT |
| | GGT-3′MGB (SEQ ID NO: 9) |

(2) Ten nanograms of DNA (buffy coat DNA with or without bisulfite conversion, Bis-BC and BC) for each reaction was used for qPCR analysis. No template control (NTC) was used as blank control.
(3) Each of the seven primer combinations for the internal control was mixed with the primers and probes for the methylation markers in the V1 assay targeting the multiple DNA methylation markers (FAM labelled) for testing the Bis-BC, BC and NTC samples.

Each qPCR reaction contains:

| | Final | Volume for one 25 |
|---|---|---|
| Component | Concentration | μL reaction (μL) |
| KAPA PROBE FAST qPCR Master Mix (2x) | 1× | 12.5 |
| V1 primer mix for DNA methylation markers (5 μM) | 0.25 μM | 1.25 |
| V1 probe mix for DNA methylation markers (5 μM) | 0.1 uM | 0.5 |
| ACTB primer mix (5 μM) | 0.06 μM | 0.3 |
| ACTB probe (5 μM) | 0.05 μM | 0.25 |
| H₂O | / | 8.2 |
| DNA template (5 ng/μL) | / | 2 |
| Total volume | / | 25 |

(4) Cycling conditions in a PCR thermocycler (Bio-rad CFX96):

| Channel | FAM/VIC |
|---|---|
| Polymerase activation | 95° C., 3 min |
| Denaturation | 95° C., 3 s |
| Annealing | 60° C., 30 s |
| Extension | 72° C., 30 s |
| Cycle number | 45 |

(5) qPCR data acquisition and data analysis to choose the best internal control assay.

### 2. Results

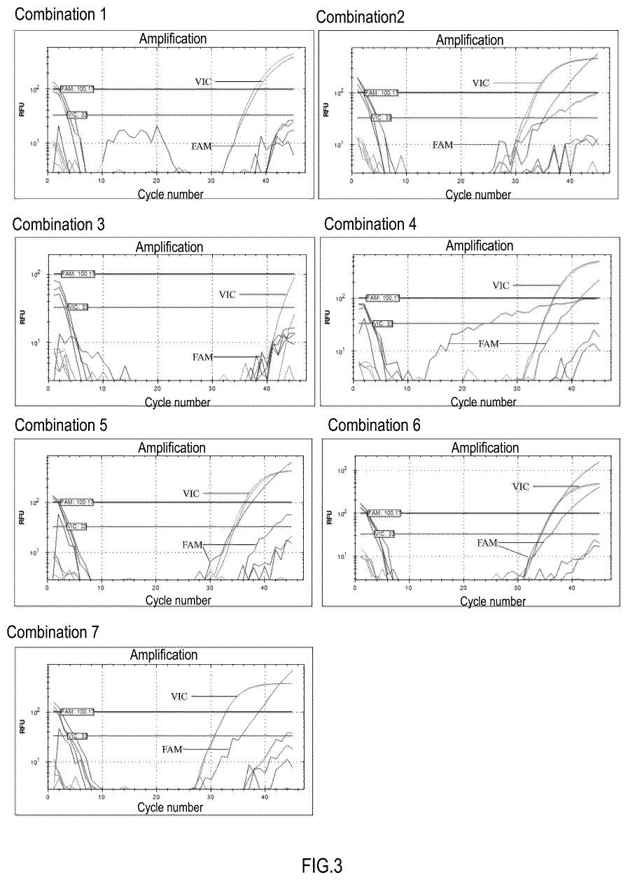

The amplification curves for the seven primer combinations are shown in FIG. 3. The corresponding results are shown below:

| | | | Average | VIC | Average |
|---|---|---|---|---|---|
| Combination | Sample | FAM Cq | FAM Cq | Cq | VIC Cq |
| 1 | NTC1 | N/A | N/A | N/A | N/A |
| | Bis-BC | N/A | N/A | 36.05 | 35.83 |
| | Bis-BC | N/A | | 35.60 | |
| | BC | N/A | N/A | N/A | N/A |
| | BC | N/A | | N/A | |
| 2 | NTC2 | N/A | N/A | N/A | N/A |
| | Bis-BC | N/A | 38.20 | 31.46 | 31.79 |
| | Bis-BC | 38.20 | | 32.12 | |
| | BC | N/A | N/A | N/A | N/A |
| | BC | N/A | | N/A | |
| 3 | NTC3 | N/A | N/A | N/A | N/A |
| | Bis-BC | N/A | N/A | N/A | 42.73 |
| | Bis-BC | N/A | | 42.73 | |
| | BC | N/A | N/A | N/A | N/A |
| | BC | N/A | | N/A | |
| 4 | NTC4 | N/A | N/A | N/A | N/A |
| | Bis-BC | N/A | 41.45 | 34.65 | 34.59 |
| | Bis-BC | 41.45 | | 34.52 | |
| | BC | N/A | 44.65 | N/A | N/A |
| | BC | 44.65 | | N/A | |
| 5 | NTC5 | N/A | N/A | N/A | N/A |
| | Bis-BC | N/A | 37.56 | 34.13 | 34.36 |
| | Bis-BC | 37.56 | | 34.58 | |
| | BC | N/A | N/A | N/A | N/A |
| | BC | N/A | | N/A | |
| 6 | NTC6 | N/A | N/A | N/A | N/A |
| | Bis-BC | 38.97 | 37.58 | 34.04 | 34.15 |
| | Bis-BC | 36.18 | | 34.26 | |
| | BC | N/A | N/A | N/A | N/A |
| | BC | N/A | | N/A | |
| 7 | NTC7 | N/A | N/A | N/A | N/A |
| | Bis-BC | 38.72 | 38.72 | 30.62 | 30.57 |
| | Bis-BC | N/A | | 30.52 | |
| | BC | N/A | N/A | N/A | N/A |
| | BC | N/A | | N/A | |

An ideal internal control assay needs to satisfy the following: (1) no non-specific signal for VIC in BC and NTC samples; (2) appropriate VIC signal in the Bis-BC sample to reflect input DNA amount; (3) does not interfere with the DNA methylation markers to create non-specific FAM signal in the Bis-BC, BC and NTC samples; and (4) does not attenuate the FAM signal for the DNA methylation markers. Based on the results above, the best primer combination for the internal assay is combination 1 with the forward primer containing a T to A mutation at the second last position at the 3′ and the reverse primer contains no mutation.

The primers and probe for the internal control assay are thus the following (the mutant base is underlined):

| Name | Sequence (5′-3′) |
|---|---|
| ACTB_F | GAGGGAGGAAGTTATGGTAGGTTAT (SEQ ID NO: 1) |
| ACTB_R | TCCTAACCACCTTCTCAACCTTAAA (SEQ ID NO: 2) |
| ACTB_probe | 5′VIC-AGAAGGTAGTTTGAAGTTGGT-3′MGB |
| | (SEQ ID NO: 9) |

### Example 3

### Establishment of Quality Control Samples

Negative and positive control samples were used for quality control of mqMSP reactions. The quality control samples were co-processed and analyzed with each batch of cfDNA samples. The test results for the quality control samples were used to evaluate whether the experiment was successful and the credibility of the results.

The positive control sample was a mixture of HCT15 CRC cell line and human buffy coat DNA (mixed at a 1:99 ratio). The negative control was human buffy coat DNA. For each reaction, 20 ng of positive or negative control sample was used.

### 1. Methods

(1) DNA samples were prepared from HCT15 CRC cancer cell line and human buffy coat cells. DNA concentrations were quantified by Qubit and DNA integrity was checked by agarose gel electrophoresis.
(2) Preparation of positive control sample: Mix 5 ng HCT15 DNA and 495 ng of buffy coat DNA, add water to a final volume of 50 μL (final concentration, 10 ng/μL). Aliquot and store the DNA samples at −80° C. Use 20 ng for each reaction.
(3) Preparation of negative control sample: Dilute a total of 500 ng buffy coat to 10 ng/μL. Aliquot and store the DNA samples at −80° C. Use 20 ng for each reaction.
(4) When processing each batch of clinical samples for bisulfite conversion and qPCR analysis, a positive and a negative control sample were processed in parallel with the same conditions. Each sample after bisulfite conversion was equally aliquoted into two for duplicate qPCR analysis of 45 cycles. The FAM and VIC Cq values were tabulated for the duplicate qPCR reactions.

### 2. Results

Different interpretation algorithms for different mqMSP assays are summarized in the following tables. + represents Cq≤45, − represents no amplification, ΔCq=VIC(average Cq)−FAM(average Cq).

When the results for both the positive and negative control samples met the conditions summarized in the following tables, this batch of qPCR reactions were considered reliable.

### V1 Assay:

| Results for controls | FAM Cq | VIC Cq | Additional requirement |
|---|---|---|---|
| Reliable positive control | FAM (+/+) | VIC (+/+) | ΔCq ≥ −1 |
| Reliable negative control | FAM(−/−)or FAM(+/−)or FAM | VIC (+/+) | ΔCq < −1 |
| | (+/+) | | |

### V2/V3/V4 Assay:

| Results for controls | FAM Cq | VIC Cq |
|---|---|---|
| Reliable positive control | FAM (+/+) | VIC (+/+) |
| Reliable negative control | FAM (−/−) | VIC (+/+) |

### Example 4

Four Combinations of Biomarkers for mqMSP (V1, V2, V3 and V4)

A total of 17 candidate DNA methylation regions (listed in Table 3) were identified by MALDI-TOF mass spectrometric analysis of multiple regions with SEPTIN9 and other genes. PCR primers for these regions were designed for quantitative methylation specific PCR (qMSP) analysis.

TABLE 3
Biomarkers and their genomic locations

| Gene | Biomarker name | Chromosome location |
|---|---|---|
| SEPTIN9 | MBSF9 | chr17: 77373456-77373518 |
| SEPTIN9 | MBSF10 | chr17: 77373564-77373617 |
| SEPTIN9 | MBSF15 | chr17: 77373973-77374049 |
| SEPTIN9 | MBSR5 | chr17: 77373985-77374054 |
| SEPTIN9 | MBSR6 | chr17: 77373914-77373986 |
| SEPTIN9 | MBSR7 | chr17: 77373843-77373898 |
| SEPTIN9 | MBSR8 | chr17: 77373747-77373824 |
| SEPTIN9 | MBSR9 | chr17: 77373691-77373745 |
| SEPTIN9 | MBSR11 | chr17: 77373520-77373600 |
| SEPTIN9 | MBSR16 | chr17: 77373100-77373185 |
| SEPTIN9 | MBSF8 | chr17: 77373359-77373422 |
| SEPTIN9 | MBSR13 | chr17: 77373361-77373438 |
| SEPTIN9 | RD1 | chr17: 77373438-77373528 |
| SEPTIN9 | RD2 | chr17: 77373384-77373452 |
| NDRG4 | NDRG4 | chr16: 58463491-58463554 |
| QKI | QKI | chr6: 163415625-163415707 |
| NPY | NPY | chr7: 24284105-24284197 |
| ACTB | ACTB | chr7: 5536826-5536901 |

The candidate DNA methylation markers were tested in multiple clinical samples. Multiple combinations including V1, V2, V3 and V4 were also tested (see Examples 5-9). The established mqMSP methods were 10 times more sensitive than a single marker assay. Data analysis algorithms were established. To be included in a combination, a biomarker must follow certain rules including: no or very low background signal in buffy coat; significantly higher methylation levels in tumor tissues than in the surrounding normal tissues; different markers complement each other in different samples to ensure sensitivity and specificity; no or very low non-specific signal derived from interference from detecting different markers. These biomarkers were further validated in multiple samples with mqMSP and NGS analysis of the mqMSP amplification products with cfDNA. Additionally, markers may be divided into three or more groups using different fluorescence channels. Example 11 will also demonstrate double and triple fluorescence designs. In triple fluorescence design, fluorescence 1 and 2 were used for DNA methylation markers while fluorescence 3 was used for the internal control.

TABLE 4
Primers and probes used in four combinations
of mqMSP analyses, with mutant bases underlined.

| Name | Sequence (5′-3′) |
|---|---|
| MBSF9_F | TTCGTCGTTGTTTTTCGC (SEQ ID NO: 10) |
| MBSF9_R | GTTAACCGCGAAATCCG (SEQ ID NO: 11) |
| MBSF9-probe | 5′FAM-AACAACGAATCGCGC-3′MGB |
| | (SEQ ID NO: 12) |
| MBSF10_F | GTTGGTTGTTGCGGTC (SEQ ID NO: 13) |
| MBSF10_R | GCCAAACCCACCCG (SEQ ID NO: 14) |
| MBSF10-probe | 5′FAM-TCCAACACGTCCGC-3′MGB |
| | (SEQ ID NO: 15) |
| MBSF15_F | TTTCGTTTGGATTCGGTAAC (SEQ ID NO: 16) |
| MBSF15_R | CCCGAACAAAACGCG (SEQ ID NO: 17) |
| MBSF15-probe | 5′FAM-TTTATTTTCGATTGAGTGGAT-3′MGB |
| | (SEQ ID NO: 18) |
| MBSR5_F | GTTTATTCGAGTAGGACGC (SEQ ID NO: 19) |
| MBSR5_R | CCGACAACGAAATAAAAAAATCG |
| | (SEQ ID NO: 20) |
| MBSR5-probe | 5′FAM-TTATTTAGTCGGAGGTGAGGA-3′MGB |
| | (SEQ ID NO: 21) |
| MBSR6_F | GGGTTTAAGCGGGGTTC (SEQ ID NO: 22) |
| MBSR6_R | ATTTCACTCTAAAAAATCCATCG |
| | (SEQ ID NO: 23) |
| MBSR6-probe | 5′FAM-TCCCCGACGACTCT-3′MGB |
| | (SEQ ID NO: 24) |
| MBSR7_F | GTTTCGAGGTAGTTTCGC (SEQ ID NO: 25) |
| MBSR7_R | TTATATTTACCAAACGACAACG |
| | (SEQ ID NO: 26) |
| MBSR7-probe | 5′FAM-CTCGAAAACTCGCGAAA-3′MGB |
| | (SEQ ID NO: 27) |
| MBSR8_F | AAATTTAGGCGGTAGTGC (SEQ ID NO: 28) |
| MBSR8_R | CCTATTAAAAACACCCGCG (SEQ ID NO: 29) |
| MBSR8-probe | 5′FAM-CTACGCGCCCTCACAA-3′MGB |
| | (SEQ ID NO: 30) |
| MBSR9_F | TTTTTTACGTAGGCGGC (SEQ ID NO: 31) |
| MBSR9_R | CCGCTAAAAACGCCG (SEQ ID NO: 32) |
| MBSR9-probe | 5′FAM-CCCGTACCCGCGCC-3′MGB |
| | (SEQ ID NO: 33) |
| MBSR11_F | GGTTTTTTTTAGTACGTTCGC |
| | (SEQ ID NO: 34) |
| MBSR11_R | CGCAACTAAATAAAATCATTTCG |
| | (SEQ ID NO: 35) |
| MBSR11-probe | 5′FAM-TAAACTAACTACTACGACCGC-3′MGB |
| | (SEQ ID NO: 36) |
| MBSR16_F | GGGGTTCGAGTGGTC (SEQ ID NO: 37) |
| MBSR16_R | CGTCCCCTAAACGCG (SEQ ID NO: 38) |
| MBSR16-probe | 5′FAM-TATACGATCGGAGCGTTT-3′MGB |
| | (SEQ ID NO: 39) |
| MBSF8_F | GTTTAGGGGTTTTTTCGGC (SEQ ID NO: 40) |
| MBSF8_R | CTAACTAAAACGCCGCG (SEQ ID NO: 41) |
| MBSF8-probe | 5′FAM-TAGTTTTGTATTGTAGGAGCGC-3′MGB |
| | (SEQ ID NO: 42) |
| MBSR13_F | GTTCGGGTTTTGCGC (SEQ ID NO: 43) |
| MBSR13_R | CTAAAAACTCCTCCGACG (SEQ ID NO: 44) |
| MBSR13-probe | 5′FAM-ACGCGAACGCGACGC-3′MGB |
| | (SEQ ID NO: 45) |
| NDRG4_F | CGGTTTTCGTTCGTTTTTTCG |
| | (SEQ ID NO: 46) |
| NDRG4_R | GTAACTTCCGCCTTCTACGC (SEQ ID NO: 47) |
| NDRG4_probe | 5′FAM-CTAAAATACCCGATAAAC-3′MGB |
| | (SEQ ID NO: 48) |
| QKI-F | TTTAGTTTCGGCGGTTATATTTTC |
| | (SEQ ID NO: 49) |
| QKI-R | CTACTCTCGAAAAAACTCGACG |
| | (SEQ ID NO: 50) |
| QKI_probe | 5′FAM-CACCGAATCCGCGCA-3′MGB |
| | (SEQ ID NO: 51) |
| RD1_F | TTAGTTGCGCGTTGATC (SEQ ID NO: 52) |
| RD1_R | CCCCGCCGAAAACG (SEQ ID NO: 53) |
| RD1-probe | 5′FAM-TAGCGGGTCGCGC-3′MGB |
| | (SEQ ID NO: 54) |
| RD2_F | TTTTGTATTGTAGGAGCGC (SEQ ID NO: 55) |
| RD2_R | ACGCCCCCGACG (SEQ ID NO: 56) |
| RD2-probe | 5′FAM-TAGGGTTCGGGTTTC-3′MGB |
| | (SEQ ID NO: 57) |
| NPY_F | GGAGTTATTTAAGCGTGATTGTTC |
| | (SEQ ID NO: 58) |
| NPY_R | AATAAAATACAAAAAACGAATCGCG |
| | (SEQ ID NO: 59) |
| NPY_probe | 5′FAM-AAACTTCCTCGCCGCGA-3′MGB |
| | (SEQ ID NO: 60) |
| ACTB_F | GAGGGAGGAAGTTATGGTAGGTTAT |
| | (SEQ ID NO: 1) |
| ACTB_R | TCCTAACCACCTTCTCAACCTTAAA |
| | (SEQ ID NO: 2) |
| ACTB_probe | 5′VIC-AGAAGGTAGTTTGAAGTTGGT-3′MGB |
| | (SEQ ID NO: 9) |

### Example 5

### Evaluation of V1 Assay for Specificity and Sensitivity

Based on the principles for inclusion in a combination of biomarkers, 10 markers (MBSF9, MBSF10, MBSF15, MBSR5, MBSR6, MBSR7, MBSR8, MBSR9, MBSR11 and MBSR16) with MGB probes were combined into a single multiplex detection.

The ACTB assay was used in the multiplex detection as internal quality control. Reaction conditions were optimized to increase the detection sensitivity. The resulting V1 assay included the following assays: MBSF9, MBSF10, MBSF15, MBSR5, MBSR6, MBSR7, MBSR8, MBSR9, MBSR11, MBSR16 and ACTB).

### 1. Methods

(1) CRC tumor tissues, surrounding normal tissues and buffy coat were used for genomic DNA extraction and bisulfite conversion.
(2) The biomarkers including MBSF9, MBSF10, MBSF15, MBSR5, MBSR6, MBSR7, MBSR8, MBSR9, MBSR11, MBSR16 and ACTB were used for analysis. Their primer and probe sequences were listed in the table in example 4.
(3) Preparation of primer and probe mixtures for qPCR:

Each primer was initially set at 200 μM, and mixed as the following:

| V1 assay primers for DNA methylation markers | volume |
|---|---|
| MBSF9 forward and reverse primers | 10 μL each |
| MBSF10 forward and reverse primers | 10 μL each |
| MBSF15 forward and reverse primers | 10 μL each |
| MBSR5 forward and reverse primers | 10 μL each |
| MBSR6 forward and reverse primers | 10 μL each |
| MBSR7 forward and reverse primers | 10 μL each |
| MBSR8 forward and reverse primers | 10 μL each |
| MBSR9 forward and reverse primers | 10 μL each |
| MBSR11 forward and reverse primers | 10 μL each |
| MBSR16 forward and reverse primers | 10 μL each |
| DNase/RNase free water | 200 μL |

| Internal control primers | Volume |
|---|---|
| ACTB forward and reverse primers | 10 μL each |
| DNase/RNase free water | 380 μL |

Each probe was initially set as 100 μM, and mixed as the following:

| V1 assay probes for DNA methylation markers | volume |
|---|---|
| MBSF9 probe | 10 μL |
| MBSF10 probe | 10 μL |
| MBSF15 probe | 10 μL |
| MBSR5 probe | 10 μL |
| MBSR6 probe | 10 μL |
| MBSR7 probe | 10 μL |
| MBSR8 probe | 10 μL |
| MBSR9 probe | 10 μL |
| MBSR11 probe | 10 μL |
| MBSR16 probe | 10 μL |
| DNase/RNase free water | 100 μL |

| Internal control probe | volume |
|---|---|
| ACTB probe | 10 μL |
| DNase/RNase free water | 190 μL |

(4) The reaction components for qPCR

| | | Volume for one 25 |
|---|---|---|
| Component | Final concentration | μL reaction (μL) |
| KAPA PROBE FAST qPCR Master Mix (2×) | 1× | 12.5 |
| Primer mix for DNA methylation markers (5 μM) | 0.25 μM | 1.25 |
| Probe mix for DNA methylation markers (5 μM) | 0.1 μM | 0.5 |
| ACTB primer mix (5 μM) | 0.06 μM | 0.3 |
| ACTB probe (5 μM) | 0.05 μM | 0.25 |
| H₂O | / | 0.2 |
| DNA template | / | 10 |
| Total volume | / | 25 |

qPCR Thermocycling Condition:

| Channel | FAM/VIC |
|---|---|
| Polymerase activation | 95° C., 3 min |
| Denaturation | 95° C., 3 s |
| Annealing | 60° C., 30 s |
| Extension | 72° C., 30 s |
| Cycle number | 45 |

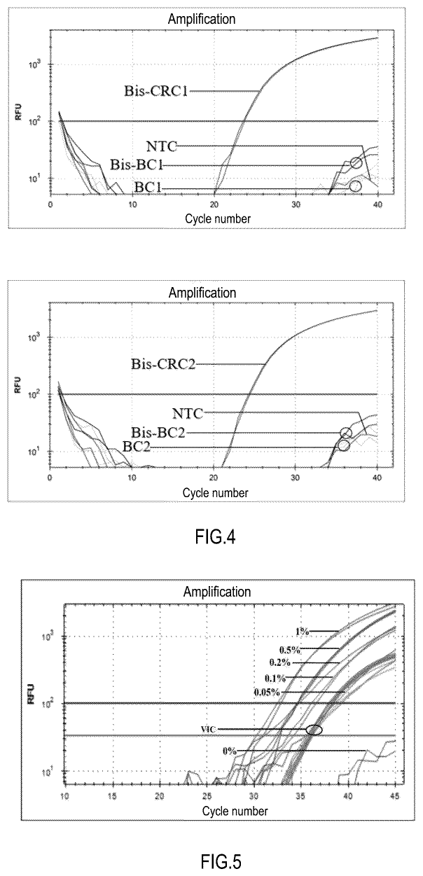

(5) Evaluation of specificity: No template control, buffy coat DNA without bisulfite conversion (BC), buffy coat DNA with bisulfite conversion (Bis-BC), and CRC tumor tissue DNA with bisulfite conversion (Bis-CRC) were analyzed by the V1 mqMSP assay. For each type of sample, 40 ng DNA was added as template. The results are shown in FIG. 4.
(6) Evaluation of sensitivity: Bisulfite converted CRC tissue DNA (as the methylated DNA for the biomarkers) and bisulfite converted buffy coat DNA (as the unmethylated DNA for the biomarkers) were mixed at various different ratios (1%, 0.5%, 0.2%, 0.1%, 0.05% and 0% of methylated DNA) to mimic samples of variable DNA methylation levels. A total of 10 ng DNA for each DNA mixture was used for mqMSP. The results are shown in FIG. 5.

### 2. Results

(1) As shown in FIG. 4, no amplification signal was produced for no template control, buffy coat DNA without bisulfite conversion (BC), or buffy coat DNA with bisulfite conversion (Bis-BC). Only CRC tumor tissue DNA with bisulfite conversion (Bis-CRC) produced clear specific amplification signal. Such results were reproduced in two sets of samples, suggesting good specificity of the assay.

The corresponding data are also shown below:

| Sample | FAM Cq |
|---|---|
| BC1 | N/A |
| BC1 | N/A |
| Bis-BC1 | N/A |
| Bis-BC1 | N/A |
| Bis-CRC1 | 23.49 |
| Bis-CRC1 | 23.65 |
| BC2 | N/A |
| BC2 | N/A |
| Bis-BC2 | N/A |
| Bis-BC2 | N/A |
| Bis-CRC2 | 24.26 |
| Bis-CRC2 | 24.4 |
| NTC | N/A |

(2) As shown in FIG. 5, Cq values for the amplification curves increased with decreasing methylation levels. Amplification curves for samples with different DNA methylation levels (1%, 0.5%, 0.2%, 0.1%, 0.05% and 0%) were from left to right and distinguishable. The most left (strongest) FAM signal was from the sample with 1% methylation, while the most right (weakest) FAM signal was from the sample with 0% methylation. The results here demonstrated that the current method can detect a sample with methylation level as low as 0.05%.

The corresponding data are also shown below:

| Sample | FAM Cq | VIC Cq |
|---|---|---|
| 1% | 32.78 | 35.50 |
| 1% | 32.40 | 36.00 |
| 1% | 32.04 | 35.56 |
| 0.5% | 34.50 | 35.69 |
| 0.5% | 34.52 | 35.90 |
| 0.5% | 34.08 | 35.84 |
| 0.2% | 34.72 | 36.02 |
| 0.2% | N/A | 36.01 |
| 0.2% | 34.51 | 35.58 |
| 0.1% | 36.04 | 35.86 |
| 0.1% | 36.51 | 35.98 |
| 0.1% | 36.98 | 35.94 |
| 0.1% | 39.25 | 35.99 |
| 0.05% | 39.23 | 36.17 |
| 0.05% | 38.55 | 36.12 |
| 0.05% | 35.71 | 35.92 |
| 0.05% | 36.88 | 35.88 |
| 0% | NA | 36.08 |
| 0% | NA | 36.07 |
| 0% | NA | 36.11 |

### Example 6

### Comparison of Sensitivity Between Single Marker and Multiple Marker Detection

### 1. Methods

(1) Primers and probe for a single marker were:

### DNA Methylation Primers and Probe

| Name | Sequence (5′-3′) |
|---|---|
| MBSF9_F | TTCGTCGTTGTTTTTCGC (SEQ ID NO: 10) |
| MBSF9_R | GTTAACCGCGAAATCCG (SEQ ID NO: 11) |
| MBSF9-probe | 5′FAM-AACAACGAATCGCGC-3′MGB |
| | (SEQ ID NO: 12) |

Primers and probe for the internal control gene: with the mutant base underlined

| Name | Sequence (5′-3′) |
|---|---|
| ACTB_F | GAGGGAGGAAGTTATGGTAGGTTAT (SEQ ID NO: 1) |
| ACTB_R | TCCTAACCACCTTCTCAACCTTAAA (SEQ ID NO: 2) |
| ACTB_probe | 5′VIC-AGAAGGTAGTTTGAAGTTGGT-3′MGB |
| | (SEQ ID NO: 9) |

The reaction components were:

| | Initial | |
|---|---|---|
| Component | concentration | Volume |
| KAPA PROBE FAST qPCR Master Mix | 2× | 12.5 μL |
| MBSF9 primer mix | 5 μM | 1.25 μL |
| MBSF9_probe | 5 μM | 0.5 μL |
| ACTB primer mix | 5 μM | 0.3 μL |
| ACTB_probe | 5 μM | 0.25 μL |
| DNA sample | / | 10 μL |
| DNase/RNase free water | / | Add to a final |
| | | volume of 25 μL |

The thermocycling condition was:

| Channel | FAM/VIC |
|---|---|
| Polymerase activation | 95° C., 3 min |
| Denaturation | 95° C., 3 s |
| Annealing | 60° C., 30 s |
| Extension | 72° C., 30 s |
| Cycle number | 45 |

(2) The multiplex detection method for multiple DNA methylation markers was as described in example 5 (V1 mqMSP assay).
(3) Bisulfite-converted CRC tumor tissue DNA and bisulfite-converted buffy coat DNA were mixed at a 1:99 ratio (1% methylation assuming tumor tissue DNA is fully methylated and buffy coat DNA is fully unmethylated for the methylation markers). For the same sample, 10 ng DNA was used for both single-marker and multiple-marker detections.

### 2. Results

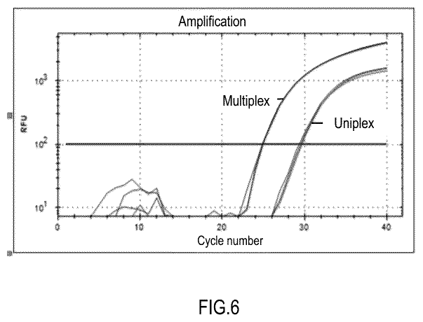

As shown in FIG. 6, there was a Cq difference of about 3-4 between the single-marker and multiple-marker assays for the same sample, suggesting approximately 10 times higher sensitivity for multiplex detection than the single marker detection.

### Example 7

### Establishment and Validation of the V2 Assay

Different combinations of DNA methylation markers may perform differently. To explore assays with better sensitivity and specificity, we designed and validated the V2 assay based on what we learned from the V1 assay. The main steps were:
(1) Based on the literature reports and our own data, multiple DNA methylation markers including SEPTIN9, NDRG4, QKI, NPY, etc were selected as candidate DNA methylation markers and qMSP primers and MGB probes were designed for each region.
(2) Each candidate DNA methylation marker was tested by probe-based qPCR in multiple sample types (multiple tumor tissues, normal tissues and buffy coat).
(3) Analyze results for each biomarker in the multiple sample types. Based on the principles for combining multiple biomarkers, we selected MBSF9, MBSR16, MBSF8, MBSR13, NDRG4, NPY, QKI for one multiplex detection. FAM fluorescence probes were used for these methylation markers, while a VIC probe was used for the internal control ACTB. We tested cfDNA from multiple different groups (CRC, polyps, advanced adenoma patients, and healthy controls) and quality control samples for mqMSP.
(4) The above multiplex assay generated false positive signals for healthy controls, negative quality control, and no template control. We used no template control to test different combinations of biomarkers to tease out the biomarker responsible for the non-specific signal.
(5) After removing the NPY assay responsible for the non-specific signal, the V2 assay including MBSF9, MBSR16, MBSF8, MBSR13, NDRG4, QKI and ACTB was established.

Details are provided below:

### 1. Methods

(1) Based on the literature reports and our own data, multiple DNA methylation markers including SEPTIN9, NDRG4, QKI, NPY, etc were selected as candidate DNA methylation markers and qMSP primers and MGB probes were designed for each region.
(2) DNA samples were prepared from tumor tissue (T), normal tissue (N) and buffy coat (B). For each sample, 1 μg of DNA was used for bisulfite conversion.
(3) Probe-based qPCR analyses were performed with the bisulfite converted tumor DNA (T), normal tissue DNA (N) and buffy coat DNA (B), using the same reaction and thermocycling condition as the V1 assay (as specified in example 5).
(4) Based on the results above and according to the principles for combining multiple biomarkers, we selected MBSF9, MBSR16, MBSF8, MBSR13, NDRG4, NPY, QKI for one multiplex detection. The methylation markers were detected by FAM fluorescence, while the internal control ACTB was detected by VIC fluorescence. We tested cfDNA from multiple different groups (CRC, polyps, advanced adenoma patients, and healthy controls) and quality control samples for mqMSP.
(5) The above multiplex assay generated false positive signals for healthy controls, negative quality control, and no template control. We used no template control to tease out the biomarker(s) responsible for the non-specific signal. The combinations tested were:

| Combination | Biomarkers |
|---|---|
| 1 | Target 1: MBSF9, MBSR16, MBSF8, MBSR13, NDRG4, |
| | NPY |
| | Target 2: ACTB |
| 2 | Target 1: MBSF9, MBSR16, MBSF8, MBSR13, NDRG4, |
| | QKI |
| | Target 2: ACTB |
| 3 | Target 1: MBSF9, MBSR16, MBSF8, MBSR13, NPY, QKI |
| | Target 2: ACTB |
| 4 | Target 1: MBSF9, MBSR16, MBSF8, NDRG4, NPY, QKI |
| | Target 2: ACTB |
| 5 | Target 1: MBSF9, MBSR16, MBSR13, NDRG4, NPY, QKI |
| | Target 2: ACTB |

(6) Based on the test results above, we identified that the non-specific signal was derived from NPY. We further compared with sensitivity of the combinations with and without NPY using the 1% methylation DNA, no significant difference was observed.
(7) Accordingly, after removing NPY, the V2 assay including MBSF9, MBSR16, MBSF8, MBSR13, NDRG4, QKI and ACTB was established. The sequences for primers and probes were listed in the table in example 4.
(8) Preparations of primer and probe mixtures for the V2 assay:

Each primer was initially set at 200 μM, and mixed as the following:

| V2 assay primers for DNA methylation markers | volume |
|---|---|
| MBSF9 forward and reverse primers | 10 μL each |
| MBSF8 forward and reverse primers | 10 μL each |
| MBSR13 forward and reverse primers | 10 μL each |
| MBSR16 forward and reverse primers | 10 μL each |
| NDRG4 forward and reverse primers | 10 μL each |
| QKI forward and reverse primers | 10 μL each |
| DNase/RNase free water | 40 μL |

| Internal control primers | Volume |
|---|---|
| ACTB forward and reverse primers | 10 μL each |
| DNase/RNase free water | 380 μL |

Each probe was initially set as 100 μM, and mixed as the following:

| V2 assay probes for | |
|---|---|
| DNA methylation markers | volume |
| MBSF9 probe | 10 μL |
| MBSF8 probe | 10 μL |
| MBSR13 probe | 10 μL |
| MBSR16 probe | 10 μL |
| NDRG4 probe | 10 μL |
| QKI probe | 10 μL |
| DNase/RNase free water | 40 μL |

| Internal control probe | volume |
|---|---|
| ACTB probe | 10 μL |
| DNase/RNase free water | 190 μL |

(9) The reaction components for qPCR

| | | Volume for one 25 |
|---|---|---|
| Component | Final concentration | μL reaction (μL) |
| KAPA PROBE FAST qPCR Master Mix (2x) | 1× | 12.5 |
| Primer mix for DNA methylation markers (12.5 μM) | 0.25 μM | 0.5 |
| Probe mix for DNA methylation markers (10 μM) | 0.1 μM | 0.25 |
| ACTB primer mix (5 μM) | 0.06 μM | 0.3 |
| ACTB probe (5 μM) | 0.05 μM | 0.25 |
| 50x ROX Low | 1× | 0.5 |
| DNA template | / | 10 |
| H₂O | | 0.7 |
| Total volume | / | 25 |

qPCR Thermocycling Condition:

| Channel | FAM/VIC |
|---|---|
| Polymerase activation | 95° C., 3 min |
| Denaturation | 95° C., 3 s |
| Annealing | 60° C., 30 s |
| Extension | 72° C., 30 s |
| Cycle number | 45 |

### 2. Results

(1) Individual DNA methylation markers were tested in multiple DNA sample types (multiple tumor tissue DNA (T), normal tissue DNA (N) and buffy coat DNA (B)). Data for some of the markers are summarized below. Sample P represents a positive DNA sample with 100% DNA methylation. DNA methylation levels were represented by ΔCq, where ΔCq=VIC(average Cq)−FAM(average Cq).

### The Results for the Tumor Samples are Summarized Below:

| | RD1 | RD2 | MBSF8 | MBSR13 | NDRG4 | QKI | MBSF9 | MBSR16 |
|---|---|---|---|---|---|---|---|---|
| Sample ID | ΔCq | ΔCq | ΔCq | ΔCq | ΔCq | ΔCq | ΔCq | ΔCq |
| P | 2.25 | 1.50 | / | / | / | / | 7.12 | 5.45 |
| T1 | / | / | 2.83 | 3.51 | 2.95 | 5.75 | 4.98 | 3.63 |
| T2 | / | / | 1.43 | 2.07 | 4.51 | 6.42 | 4.95 | −10.99 |
| T3 | / | / | 2.41 | 2.93 | 4.03 | 4.96 | 5.34 | 2.55 |
| T4 | / | / | 1.48 | 2.14 | −0.33 | 4.46 | 3.60 | 1.71 |
| T5 | / | / | 3.26 | 4.00 | −2.98 | −5.64 | 5.80 | 0.84 |
| T6 | / | / | −3.20 | −3.16 | −3.48 | 0.48 | −0.91 | −11.38 |
| T7 | / | / | 0.47 | 1.39 | 1.80 | 2.73 | 2.99 | −2.90 |
| T8 | / | / | 1.28 | 2.16 | 4.35 | 6.43 | 5.50 | −11.45 |
| T9 | / | / | −4.55 | −3.32 | −10.73 | −10.69 | −1.79 | −3.72 |
| T10 | / | / | 3.06 | 4.02 | 4.41 | 5.14 | 5.60 | 4.27 |
| T11 | 3.76 | 3.73 | / | / | 4.25 | 4.50 | 5.05 | 3.55 |
| T12 | 3.84 | 4.09 | / | / | 3.49 | 6.19 | 4.95 | −11.35 |
| T13 | 3.33 | 3.43 | / | / | 3.90 | 4.76 | 4.53 | 3.65 |
| T14 | 3.52 | 3.49 | / | / | 3.77 | 5.28 | 4.84 | 3.67 |
| T15 | −1.41 | 0.65 | / | / | −1.39 | 4.12 | 1.52 | −10.74 |
| T16 | 3.47 | 3.51 | / | / | / | / | 4.77 | 3.57 |
| T17 | 4.02 | 3.99 | / | / | / | / | 5.51 | 0.06 |
| T18 | −9.14 | 3.56 | / | / | / | / | 0.66 | −11.25 |
| T19 | 2.42 | 2.28 | / | / | / | / | 2.97 | −11.20 |
| T20 | 4.99 | 5.23 | / | / | / | / | 5.87 | −3.95 |
| T21 | / | / | / | / | / | / | 6.21 | 4.58 |
| T22 | / | / | / | / | / | / | 4.79 | 3.60 |
| T23 | / | / | / | / | / | / | 6.48 | −9.58 |
| T24 | / | / | / | / | / | / | 4.65 | 3.78 |
| T25 | / | / | / | / | / | / | −0.41 | −2.69 |
| T26 | / | / | / | / | / | / | 7.14 | 5.14 |
| T27 | / | / | / | / | / | / | 4.97 | 3.42 |
| T28 | / | / | / | / | / | / | 6.00 | 3.31 |
| T29 | / | / | / | / | / | / | 3.48 | −11.80 |
| T30 | / | / | / | / | / | / | 4.47 | 3.23 |
| T31 | / | / | / | / | / | / | 3.20 | −10.62 |
| T32 | / | / | / | / | / | / | 5.47 | 0.98 |
| T33 | / | / | / | / | / | / | 2.27 | −5.85 |
| T34 | / | / | / | / | / | / | 5.72 | −5.40 |
| T35 | / | / | / | / | / | / | 6.83 | 3.32 |
| T36 | / | / | / | / | 3.28 | 4.80 | 5.35 | 1.28 |
| T37 | / | / | / | / | 4.01 | 5.45 | 4.28 | 2.45 |
| T38 | / | / | / | / | 5.35 | 7.87 | 6.39 | 5.16 |
| T39 | / | / | / | / | 3.45 | 5.27 | 2.61 | −11.08 |
| T40 | / | / | / | / | 3.94 | 4.86 | 4.22 | 1.72 |

### Results for Normal Tissue Samples:

| | RD1 | RD2 | MBSF8 | MBSR13 | NDRG4 | QKI | MBSF9 | MBSR16 |
|---|---|---|---|---|---|---|---|---|
| Sample ID | ΔCq | ΔCq | ΔCq | ΔCq | ΔCq | ΔCq | ΔCq | ΔCq |
| P | 2.25 | 1.50 | / | / | / | / | 7.12 | 5.45 |
| N1 | / | / | −2.39 | −0.56 | −2.10 | 2.60 | 0.41 | −11.24 |
| N2 | / | / | −1.79 | −1.12 | −2.03 | 0.36 | −0.90 | −11.65 |
| N3 | / | / | −6.78 | −3.57 | −3.21 | −1.38 | −1.75 | −7.05 |
| N4 | / | / | −3.21 | −1.83 | −0.48 | 3.33 | −0.29 | −10.82 |
| N5 | / | / | −3.00 | −1.68 | −4.11 | 0.63 | −0.64 | −11.23 |
| N6 | / | / | −3.49 | −4.37 | −6.45 | −0.56 | −2.08 | −9.96 |
| N7 | / | / | −4.81 | −1.45 | −1.06 | 2.05 | −0.94 | −11.27 |
| N8 | / | / | −11.82 | −3.29 | −3.75 | 0.34 | −4.69 | −11.67 |
| N9 | / | / | −10.48 | −8.58 | −3.63 | 0.47 | −3.09 | −10.54 |
| N10 | / | / | −5.28 | −2.94 | −1.46 | 1.48 | −0.87 | −10.87 |
| N11 | −2.30 | 0.19 | / | / | −2.51 | 0.16 | 0.58 | −11.35 |
| N12 | −4.40 | −1.23 | / | / | −3.61 | 0.93 | −1.86 | −11.42 |
| N13 | −3.70 | 0.14 | / | / | −2.47 | 1.77 | 0.47 | −10.63 |
| N14 | −3.95 | 1.20 | / | / | −5.02 | 1.97 | 0.81 | −10.48 |
| N15 | −6.37 | −3.21 | / | / | −1.69 | 1.88 | −1.65 | −10.53 |
| N16 | −5.28 | −2.91 | / | / | / | / | −1.49 | −2.83 |
| N17 | −2.35 | −5.02 | / | / | / | / | −0.59 | −7.38 |
| N18 | −4.41 | −2.26 | / | / | / | / | −2.62 | −10.78 |
| N19 | −3.68 | −1.72 | / | / | / | / | −2.42 | −10.32 |
| N20 | −3.99 | −3.00 | / | / | / | / | −2.84 | −10.52 |
| N21 | / | / | / | / | / | / | −0.23 | 3.38 |
| N22 | / | / | / | / | / | / | −5.73 | −10.71 |
| N23 | / | / | / | / | / | / | −1.85 | −10.78 |
| N24 | / | / | / | / | / | / | −1.95 | −2.98 |
| N25 | / | / | / | / | / | / | 0.06 | −9.75 |
| N26 | / | / | / | / | / | / | −4.64 | −3.52 |
| N27 | / | / | / | / | / | / | −0.71 | −6.13 |
| N28 | / | / | / | / | / | / | 0.17 | −10.02 |
| N29 | / | / | / | / | / | / | −0.59 | −10.28 |
| N30 | / | / | / | / | / | / | −1.59 | −3.65 |
| N31 | / | / | / | / | / | / | −3.58 | −11.51 |
| N32 | / | / | / | / | / | / | −1.27 | −11.02 |
| N33 | / | / | / | / | / | / | −1.11 | −11.29 |
| N34 | / | / | / | / | / | / | −2.31 | −11.28 |
| N35 | / | / | / | / | / | / | −0.53 | −3.42 |
| N36 | / | / | / | / | −2.07 | 0.81 | −0.45 | −7.34 |
| N37 | / | / | / | / | −0.49 | 1.20 | −0.66 | −10.27 |
| N38 | / | / | / | / | −6.00 | −1.01 | −2.73 | −11.58 |
| N39 | / | / | / | / | −2.53 | 0.17 | −1.89 | −11.02 |
| N40 | / | / | / | / | −2.97 | −0.39 | −2.19 | −10.83 |

### Results for Buffy Coat Samples:

| | RD1 | RD2 | MBSF8 | MBSR13 | NDRG4 | QKI | MBSF9 | MBSR16 |
|---|---|---|---|---|---|---|---|---|
| Sample ID | ΔCq | ΔCq | ΔCq | ΔCq | ΔCq | ΔCq | ΔCq | ΔCq |
| P | 2.25 | 1.50 | / | / | / | / | 7.12 | 5.45 |
| B1 | / | / | −11.29 | −11.55 | −11.51 | −11.14 | −11.11 | −10.71 |
| B2 | / | / | −11.60 | −11.80 | −1.85 | −11.48 | −11.36 | −11.43 |
| B3 | / | / | −11.75 | −11.82 | −4.09 | −11.42 | −11.55 | −11.22 |
| B4 | / | / | −11.60 | −11.71 | −6.17 | −11.26 | −11.38 | −11.07 |
| B5 | −10.14 | −10.17 | / | / | / | −9.63 | −10.46 | −10.27 |
| B6 | / | / | −10.88 | −10.96 | −10.91 | −10.74 | −10.63 | −10.50 |
| B7 | / | / | −11.51 | −11.81 | −4.81 | −3.21 | −11.28 | −11.16 |
| B8 | / | / | −11.22 | −11.48 | −11.27 | −11.07 | −11.14 | −10.79 |
| B9 | / | / | −11.58 | −12.10 | −4.72 | −11.54 | −11.38 | −11.36 |
| B10 | / | / | −11.80 | −11.94 | −11.77 | −11.60 | −11.49 | −11.34 |
| B11 | −11.84 | −11.82 | −11.96 | −12.23 | −12.11 | −11.81 | −11.94 | −11.73 |
| B12 | −5.89 | −11.62 | / | / | −4.75 | −11.61 | −11.64 | −11.70 |
| B13 | −5.90 | −11.68 | / | / | −11.76 | −11.72 | −11.75 | −11.75 |
| B14 | −11.59 | −11.65 | / | / | −7.09 | −11.75 | −11.42 | −11.40 |
| B15 | −11.45 | −11.48 | / | / | −5.15 | −11.61 | −11.34 | −11.27 |
| B16 | −11.71 | −11.95 | / | / | −5.68 | −11.99 | −11.92 | −11.70 |
| B17 | −11.74 | −11.67 | / | / | −4.27 | −3.56 | −11.88 | −11.64 |
| B18 | −11.77 | −11.72 | / | / | −6.97 | −11.98 | −11.84 | −11.86 |
| B19 | −11.63 | −11.62 | / | / | −4.57 | −11.83 | −11.62 | −11.62 |
| B20 | / | / | / | / | −11.71 | −11.62 | −11.73 | −11.63 |

(2) Considering the results above and according to the principles for including biomarkers for multiplex detection, seven biomarkers including MBSF9, MBSR16, MBSF8, MBSR13, NDRG4, NPY, QKI were included in the multiplex detection assay. These markers showed no or very low background signal in buffy coat; significantly higher methylation levels in tumor tissues than the surrounding normal tissues; and different markers complement each other in different samples. The DNA methylation markers were labelled with FAM fluorescence while the internal control ACTB was labeled with VIC fluorescence. We tested cfDNA from multiple different groups (CRC, polyps, advanced adenoma patients, and healthy controls) and quality control samples with mqMSP. The results are shown below (an example of failed multiplex combination):

| | Plasma DNA | cfDNA amount | | | | |
|---|---|---|---|---|---|---|
| Sample type | ID | (ng) | Average FAM Cq | FAM Cq Std. Dev | Average VIC Cq | VIC Cq Std. Dev |
| CRC | 869R | 23.88 | 32.26 | 0.05 | 35.42 | 0.14 |
| CRC | 891R | 27.81 | 25.87 | 0.04 | 30.66 | 0.09 |
| CRC | 168R | 21.32 | 33.02 | N/A | 35.61 | N/A |
| CRC | 584R | 52.61 | 26.13 | 0.02 | 30.65 | 0.22 |
| CRC | 559R | 50.61 | 24.49 | 0.19 | 28.84 | 0.02 |
| Benign polyps | 420Y | 50.48 | 34.71 | 0.51 | 34.50 | 0.08 |
| Advanced | AA-224Y | 38.91 | 35.28 | 0.52 | 35.02 | 0.22 |
| adenoma | | | | | | |
| Healthy control | 698Y | 20.00 | 34.13 | 0.28 | 35.85 | 0.22 |
| Healthy control | 662Y | 32.07 | 35.87 | 0.41 | 35.02 | 0.19 |
| Healthy control | 661Y | 18.00 | 35.19 | 0.34 | 35.65 | 0.02 |
| Positive quality | 1% Meth DNA | 10.00 | 33.49 | 0.43 | 36.21 | 0.26 |
| control | | | | | | |
| Negative quality | BC DNA | 10.00 | 40.09 | 0.46 | 35.38 | 0.44 |
| control | | | | | | |
| Blank control | NTC | / | 40.12 | N/A | N/A | N/A |

(3) The results above showed non-specific signals from healthy controls, negative quality control and blank control. We thus performed analyses using NTC (no template control) to tease out biomarker(s) responsible for non-specific signals. The combinations tested are list below:

| Combination | Biomarkers |
|---|---|
| 1 | Target 1: MBSF9, MBSR16, MBSF8, MBSR13, NDRG4, |
| | NPY |
| | Target 2: ACTB |
| 2 | Target 1: MBSF9, MBSR16, MBSF8, MBSR13, NDRG4, |
| | QKI |
| | Target 2: ACTB |
| 3 | Target 1: MBSF9, MBSR16, MBSF8, MBSR13, NPY, QKI |
| | Target 2: ACTB |
| 4 | Target 1: MBSF9, MBSR16, MBSF8, NDRG4, NPY, QKI |
| | Target 2: ACTB |
| 5 | Target 1: MBSF9, MBSR16, MBSR13, NDRG4, NPY, QKI |
| | Target 2: ACTB |

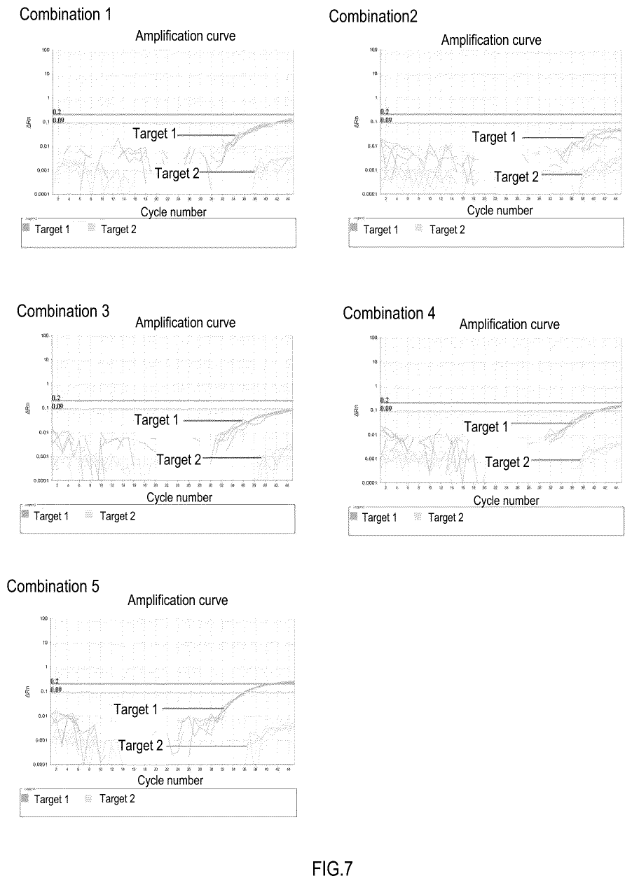

The amplification curves are shown in FIG. 7, with corresponding data summarized below:

| | FAM | Average | VIC | Average |
|---|---|---|---|---|
| Sample | Cq | FAM Cq | Cq | VIC Cq |
| Combination1-NTC | N/A | N/A | N/A | N/A |
| Combination1-NTC | N/A | | N/A | |
| Combination1-NTC | N/A | | N/A | |
| Combination1-NTC | N/A | | N/A | |
| Combination1-NTC | N/A | | N/A | |
| Combination2-NTC | N/A | N/A | N/A | N/A |
| Combination2-NTC | N/A | | N/A | |
| Combination2-NTC | N/A | | N/A | |
| Combination2-NTC | N/A | | N/A | |
| Combination2-NTC | N/A | | IN/A | |
| Combination3-NTC | N/A | N/A | N/A | N/A |
| Combination3-NTC | N/A | | N/A | |
| Combination3-NTC | N/A | | N/A | |
| Combination3-NTC | N/A | | N/A | |
| Combination3-NTC | N/A | | N/A | |
| Combination4-NTC | N/A | N/A | N/A | N/A |
| Combination4-NTC | N/A | | N/A | |
| Combination4-NTC | N/A | | N/A | |
| Combination4-NTC | N/A | | \|N/A | |
| Combination4-NTC | N/A | | N/A | |
| Combination5-NTC | 41.55 | 41.27 | N/A | N/A |
| Combination5-NTC | 41.25 | | N/A | |
| Combination5-NTC | 41.24 | | N/A | |
| Combination5-NTC | 40.66 | | N/A | |
| Combination5-NTC | 41.64 | | N/A | |

According to the results in FIG. 7. lowest background signal in combination 2 was seen in which NPY was removed, we thus reasoned that the non-specific signal was largely derived from NPY.
(4) We further compared with sensitivity of the combinations with and without NPY using the 1% methylation DNA, no significant difference was observed. The results are summarized below:

| | With NPY | | | | Without NPY | | | |
|---|---|---|---|---|---|---|---|---|
| Sample | Fluor | Cq | Average Cq | Cq Std. Dev | Fluor | Cq | Average Cq | Cq Std. Dev |
| 1% Meth DNA | FAM | 36.89 | 36.21 | 0.48 | FAM | 36.86 | 36.43 | 0.33 |
| 1% Meth DNA | FAM | 35.87 | | | FAM | 36.14 | | |
| 1% Meth DNA | FAM | 36.20 | | | FAM | 36.53 | | |
| 1% Meth DNA | FAM | 35.88 | | | FAM | 36.19 | | |
| 1% Meth DNA | VIC | 36.07 | 36.34 | 0.19 | VIC | 35.47 | 35.78 | 0.25 |
| 1% Meth DNA | VIC | 36.40 | | | VIC | 35.69 | | |
| 1% Meth DNA | VIC | 36.52 | | | VIC | 35.95 | | |
| 1% Meth DNA | VIC | 36.36 | | | VIC | 36.00 | | |

The V2 assay including MBSF9, MBSR16, MBSF8, MBSR13, NDRG4, QKI and ACTB was thus established.

### Example 8

### Establishment and Validation of the V3 Assay

Different combinations of DNA methylation markers may perform differently. To explore assays with better sensitivity and specificity, we designed and validated the V3 assay based on what we learned from the V2 assay. The main steps were:
(1) Based on the literature reports and our own data, multiple DNA methylation markers including SEPTIN9, NDRG4, QKI, NPY, SDC2, etc were selected as candidate DNA methylation markers and qMSP primers and MGB probes were designed for each region.
(2) Each candidate DNA methylation marker was tested by its respective uniplex assay in multiple sample types (multiple tumor tissues, normal tissues and buffy coat) with reference to the V2 assay.
(3) Based on the principles for combining multiple biomarkers, we established the V3 multiplex assay targeting MBSF9, MBSF8, MBSR13, NDRG4, QKI, RD1, RD2, and ACTB (internal control). Sequences for the primers and probes are listed in the table in example 4

The PCR reaction system and the thermocycling conditions were the same as the V2 assay.
(4) Testing the sensitivity and specificity of the V3 assay.

Details are provided below:

### 1. Methods

(1) Based on the literature reports and our own data, multiple DNA methylation markers including SEPTIN9, NDRG4, QKI, NPY, SDC2 etc were selected as candidate DNA methylation markers and qMSP primers and MGB probes were designed for each region.
(2) Multiple sample types (multiple tumor tissue DNA (T), normal tissue DNA (N) and buffy coat DNA (B)) were analyzed using the same reaction system and thermocycling condition as the V2 assay.
(3) Based on the principles for combining multiple biomarkers, we selected MBSF9, MBSF8, MBSR13, NDRG4, QKI, RD1, RD2, and ACTB (internal control) for the V3 assay. The sequences for the primers and probes are listed in the table in example 4.
(4) Preparations of primer and probe mixtures for the V3 assay:

Each primer was initially set at 200 μM, and mixed as the following:

| V3 assay primers for DNA methylation markers | volume |
|---|---|
| MBSF9 forward and reverse primers | 10 μL each |
| MBSF8 forward and reverse primers | 10 μL each |
| MBSR13 forward and reverse primers | 10 μL each |
| NDRG4 forward and reverse primers | 10 μL each |
| QKI forward and reverse primers | 10 μL each |
| RD1 forward and reverse primers | 10 μL each |
| RD2 forward and reverse primers | 10 μL each |
| DNase/RNase free water | 20 μL |

| Internal control primers | Volume |
|---|---|
| ACTB forward and reverse primers | 10 μL each |
| DNase/RNase free water | 380 μL |

Each probe was initially set as 100 μM, and mixed as the following:

| V3 assay probes for DNA methylation markers | volume |
|---|---|
| MBSF9 probe | 10 μL |
| MBSF8 probe | 10 μL |
| MBSR13 probe | 10 μL |
| NDRG4 probe | 10 μL |
| QKI probe | 10 μL |
| RD1 probe | 10 μL |
| RD2 probe | 10 μL |
| DNase/RNase free water | 30 μL |

| Internal control probe | volume |
|---|---|
| ACTB probe | 10 μL |
| DNase/RNase free water | 190 μL |

(5) The reaction components for qPCR

| | | Volume for |
|---|---|---|
| | Final | one 25 μL |
| Component | concentration | reaction (μL) |
| KAPA PROBE FAST | 1× | 12.5 |
| qPCR Master Mix (2×) | | |
| Primer mix for DNA methylation | 0.25 μM | 0.5 |
| markers (12.5 μM) | | |
| Probe mix for DNA methylation | 0.1 μM | 0.25 |
| markers (10 μM) | | |
| ACTB primer mix (5 μM) | 0.06 μM | 0.3 |
| ACTB probe (5 μM) | 0.05 μM | 0.25 |
| 50× ROX Low | 1× | 0.5 |
| DNA template | / | 10 |
| H₂O | | 0.7 |
| Total volume | / | 25 |

qPCR Thermocycling Condition:

| Channel | FAM/VIC |
|---|---|
| Polymerase activation | 95° C., 3 min |
| Denaturation | 95° C., 3 s |
| Annealing | 60° C., 30 s |
| Extension | 72° C., 30 s |
| Cycle number | 45 |

(6) Sensitivity of the V3 assay was analyzed using DNA samples with 1%, 0.5%, 0.2% and 0% methylation levels, with 10 ng DNA for each qPCR reaction.
(7) Specificity of the V3 assay was analyzed by using eight different buffy coat samples (610B, 624B, 630B, 642B, 646B, 662B, 671B, 625B), with 25 ng DNA for each qPCR reaction.

### 2. Results

### (1) Sensitivity Test

Among the samples with different methylation levels, the sample with 1% DNA methylation has the highest methylation level, and accordingly its fluorescence signal for the DNA methylation markers (Average FAM Cq value) was the strongest, followed by samples with 0.5% and 0.2% methylation levels. The sample with 0% methylation showed no signal for the DNA methylation markers. Taken together, the data showed that the V3 assay has good sensitivity capable of detecting methylation level as low as 0.2%.

| | | FAM | Average | VIC | Average | | Average |
|---|---|---|---|---|---|---|---|
| Sample | Replicate | Cq | FAM Cq | Cq | VIC Cq | ΔCq | ΔCq |
| 1% | 1 | 35.92 | 34.89 | 37.13 | 36.71 | 1.21 | 1.82 |
| Meth | 2 | 33.87 | | 36.29 | | 2.42 | |
| DNA | | | | | | | |
| 0.5% | 1 | 36.17 | 37.56 | 36.44 | 36.84 | 0.27 | −0.72 |
| Meth | 2 | 38.95 | | 37.23 | | −1.71 | |
| DNA | | | | | | | |
| 0.2% | 1 | 39.41 | 39.61 | 36.64 | 37.30 | −2.77 | −2.31 |
| Meth | 2 | 39.82 | | 37.96 | | −1.86 | |
| DNA | | | | | | | |
| 0% | 1 | NA | NA | 36.54 | 36.79 | NA | NA |
| Meth | 2 | NA | | 37.05 | | NA | |
| DNA | | | | | | | |

### (2) Specificity Test

Since buffy coat DNA samples are expected to be either not methylated or have low methylation levels for the chosen methylation biomarkers, the corresponding fluorescence signals for the DNA methylation markers (Average FAM Cq) are theoretically either below detection or very low. Using the V3 assay on the multiple buffy coat DNA samples, we observed low signals in all samples except for samples 610B and 624B, suggesting the V3 assay can distinguish tumor tissue DNA and buffy coat DNA with good specificity.

| | | FAM | Average | VIC | Average | | Average |
|---|---|---|---|---|---|---|---|
| Sample | Replicate | Cq | FAM Cq | Cq | VIC Cq | ΔCq | ΔCq |
| 610B | 1 | 35.62 | 35.81 | 35.15 | 35.24 | −0.48 | −0.56 |
| | 2 | 35.99 | | 35.34 | | −0.65 | |
| 624B | 1 | 37.32 | 37.45 | 34.52 | 34.51 | −2.80 | −2.94 |
| | 2 | 37.58 | | 34.50 | | −3.08 | |
| 630B | 1 | N/A | N/A | 34.31 | 34.36 | N/A | N/A |
| | 2 | N/A | | 34.41 | | N/A | |
| 642B | 1 | 37.77 | 41.04 | 34.38 | 34.18 | −3.39 | −6.86 |
| | 2 | 44.32 | | 33.99 | | −10.33 | |
| 646B | 1 | N/A | N/A | 34.93 | 35.00 | N/A | N/A |
| | 2 | N/A | | 35.07 | | N/A | |
| 662B | 1 | 42.24 | 41.35 | 34.03 | 33.96 | −8.22 | −7.39 |
| | 2 | 40.45 | | 33.88 | | −6.57 | |
| 671B | 1 | 41.06 | 41.06 | 33.95 | 33.93 | −7.11 | −7.13 |
| | 2 | N/A | | 33.92 | | N/A | |
| 625B | 1 | IN/A | N/A | 35.11 | 34.88 | N/A | N/A |
| | 2 | N/A | | 34.64 | | N/A | |

### Example 9

### Establishment and Validation of the V4 Assay

Different combinations of DNA methylation markers may perform differently. To explore assays with better sensitivity and specificity, we designed and established the V4 assay based on what we learned from the V3 assay. The main steps were:
(1) We removed the NDRG4 assay and the RD2_F primer from the V3 assay to obtain the V4 assay which includes MBSF9, MBSF8, MBSR13, QKI, RD1, RD2 (without the RD2_F primer), and ACTB (internal control). Sequences for the primers and probes are listed in the table in example 4.

The PCR reaction system and the thermocycling conditions were the same as the V2 assay.
(2) Testing the sensitivity and specificity of the V4 assay.

Details are provided below:

### 1. Methods

(1) Based on the results from the V3 assay, there were false positive signals for buffy coat DNA samples. We thus removed the NDRG4 assay and the RD2_F primer from the V3 assay to obtain the V4 assay which includes MBSF9, MBSF8, MBSR13, QKI, RD1, RD2 (without the RD2_F primer), and ACTB (internal control). Sequences for the primers and probes are listed in the table in example 4.
(2) Preparations of primer and probe mixtures for the V4 assay:

Each primer was initially set at 200 μM, and mixed as the following:

| V4 assay primers for DNA methylation markers | volume |
|---|---|
| MBSF9 forward and reverse primers | 10 μL each |
| MBSF8 forward and reverse primers | 10 μL each |
| MBSR13 forward and reverse primers | 10 μL each |
| QKI forward and reverse primers | 10 μL each |
| RD1 forward and reverse primers | 10 μL each |
| RD2 reverse primer | 10 μL |
| DNase/RNase free water | 50 μL |

| Internal control primers | Volume |
|---|---|
| ACTB forward and reverse primers | 10 μL each |
| DNase/RNase free water | 380 μL |

Each probe was initially set as 100 μM, and mixed as the following:

| V4 assay probes for DNA methylation markers | volume |
|---|---|
| MBSF9 probe | 10 μL |
| MBSF8 probe | 10 μL |
| MBSR13 probe | 10 μL |
| QKI probe | 10 μL |
| RD1 probe | 10 μL |
| RD2 probe | 10 μL |
| DNase/RNase free water | 40 μL |

| Internal control probe | volume |
|---|---|
| ACTB probe | 10 μL |
| DNase/RNase free water | 190 μL |

(3) The reaction components for qPCR:

| | Final | Volume for one 25 |
|---|---|---|
| Component | concentration | μL reaction (μL) |
| KAPA PROBE FAST qPCR | 1× | 12.5 |
| Master Mix (2×) | | |
| Primer mix for DNA methylation | 0.25 μM | 0.5 |
| markers (12.5 μM) | | |
| Probe mix for DNA methylation | 0.1 μM | 0.25 |
| markers (10 μM) | | |
| ACTB primer mix (5 μM) | 0.06 μM | 0.3 |
| ACTB probe (5 μM) | 0.05 μM | 0.25 |
| 50× ROX Low | 1× | 0.5 |
| DNA template | / | 10 |
| H₂O | | 0.7 |
| Total volume | / | 25 |

qPCR Thermocycling Condition:

| Channel | FAM/VIC |
|---|---|
| Polymerase activation | 95° C., 3 min |
| Denaturation | 95° C., 3 s |
| Annealing | 60° C., 30 s |
| Extension | 72° C., 30 s |
| Cycle number | 45 |

(4) Specificity of the V4 assay was analyzed by using eight different buffy coat samples (610B, 624B, 630B, 642B, 646B, 662B, 671B, 625B), with 25 ng DNA for each qPCR reaction.
(5) Sensitivity of the V4 assay was analyzed by using 10 DNA mixtures (10 CRC tumor tissue DNA samples from stage I or II patients mixed with buffy coat DNA from a healthy individual at a 1:9 ratio, representing samples with DNA methylation levels between 0.1-0.8%, with 10 ng DNA mixture for each qPCR reaction.

### 2. Results

### (1) Specificity Test

Since buffy coat DNA samples are expected to be either not methylated or have low methylation levels for the chosen methylation biomarkers, the corresponding fluorescence signals for the DNA methylation markers (Average FAM Cq) are theoretically either below detection or very low. Using the V4 assay on the multiple buffy coat DNA samples, we observed either no or low signals in all buffy coat samples, suggesting the V4 assay can distinguish tumor tissue DNA and buffy coat DNA with good specificity.

| | | FAM | Average | VIC | Average | | Average |
|---|---|---|---|---|---|---|---|
| Sample | Replicate | Cq | FAM Cq | Cq | VIC Cq | ΔCq | ΔCq |
| 610B | 1 | N/A | N/A | 35.05 | 35.11 | N/A | N/A |
| | 2 | N/A | | 35.18 | | N/A | |
| 624B | 1 | N/A | N/A | 34.54 | 34.54 | N/A | N/A |
| | 2 | N/A | | 34.53 | | N/A | |
| 630B | 1 | N/A | N/A | 34.15 | 34.17 | N/A | N/A |
| | 2 | N/A | | 34.19 | | N/A | |
| 642B | 1 | N/A | N/A | 34.35 | 34.18 | N/A | N/A |
| | 2 | N/A | | 34.01 | | N/A | |
| 646B | 1 | N/A | N/A | 34.67 | 34.86 | N/A | N/A |
| | 2 | N/A | | 35.05 | | N/A | |
| 662B | 1 | N/A | N/A | 33.67 | 33.71 | N/A | N/A |
| | 2 | N/A | | 33.75 | | N/A | |
| 671B | 1 | 42.37 | 42.37 | 33.77 | 33.81 | −8.59 | −8.56 |
| | 2 | N/A | | 33.84 | | N/A | |
| 625B | 1 | N/A | N/A | 34.92 | 34.71 | N/A | N/A |
| | 2 | N/A | | 34.51 | | N/A | |

### (2) Sensitivity Test

Fluorescence signals (Average FAM Cq) for the DNA methylation markers were stably detected in all mixture samples with methylation levels between 0.1-0.8%, demonstrating that the V4 assay has good sensitivity and can detect methylation levels as low as 0.1%.

| | FAM | Average | VIC | Average | Average ΔCq |
|---|---|---|---|---|---|
| Sample | Cq | FAM Cq | Cq | VIC Cq | (Cq_{VIC}-Cq_{FAM}) |
| T1 mixture | 34.92 | 35.46 | 36.34 | 37.14 | 1.67 |
| | 36.01 | | 37.93 | | |
| T2 mixture | 34.33 | 34.32 | 36.67 | 36.51 | 2.19 |
| | 34.31 | | 36.35 | | |
| T3 mixture | 31.95 | 31.88 | 35.70 | 35.59 | 3.71 |
| | 31.82 | | 35.47 | | |
| T4 mixture | 31.75 | 31.91 | 36.04 | 36.31 | 4.40 |
| | 32.07 | | 36.58 | | |
| T5 mixture | 31.28 | 31.45 | 35.66 | 36.23 | 4.79 |
| | 31.61 | | 36.80 | | |
| T6 mixture | 34.24 | 34.11 | 36.24 | 36.24 | 2.14 |
| | 33.97 | | 36.25 | | |
| T7 mixture | 32.11 | 32.14 | 35.64 | 35.51 | 3.37 |
| | 32.17 | | 35.38 | | |
| T8 mixture | 32.57 | 32.43 | 35.69 | 35.82 | 3.39 |
| | 32.29 | | 35.95 | | |
| T9 mixture | 33.23 | 33.33 | 36.04 | 36.23 | 2.90 |
| | 33.43 | | 36.43 | | |
| T10 mixture | 37.35 | 39.97 | 37.45 | 38.11 | −1.86 |
| | 42.60 | | 38.77 | | |

### Example 10

Amplicon Sequencing for cfDNA mqMSP Products

### 1. Methods

(1) To analyze the amplification efficiency and signal differences for V1 and V2 mqMSP assays in detecting cfDNA from different sample types, we performed NGS analysis for mqMSP amplification products from 83 different samples including: M.SssI treated buffy coat DNA (as 100% methylation control), 1% methylation DNA mixture sample, and plasma cfDNA samples from CRC, advanced adenoma, benign polyps, healthy individuals and volunteers. These 83 samples included 28 samples and 55 samples analyzed by V2 and V1 assays, respectively, as shown below:

| mqMSP products from | mqMSP products from |
|---|---|
| V2 assay (28) | V1 assay (55) |
| mqMSP product from M.SssI | mqMSP product from M.SssI |
| treated buffy coat DNA (1) | treated buffy coat DNA (1) |
| mqMSP products from | mqMSP products from |
| 1% Meth DNA (4) | 1% Meth DNA (4) |
| (a mixture of HCT15 CRC cell line | (a mixture of HCT15 CRC cell line |
| DNA [symbol] buffy coat DNA) | DNA [symbol] buffy coat DNA) |
| mqMSP products from | mqMSP products from |
| CRC cfDNA (20) | CRC cfDNA (28) |
| mqMSP product from advanced | mqMSP products from advanced |
| adenoma cfDNA (1) | adenoma cfDNA (7) |
| mqMSP product from | mqMSP products from |
| benign polyp cfDNA (1) | benign polyp cfDNA (6) |
| mqMSP product from | mqMSP products from healthy |
| volunteer cfDNA (1) | individual cfDNA (5) |
| | mqMSP products from |
| | volunteer cfDNA (4) |

(2) DNA library preparation and NGS analysis for the mqMSP amplification products: The mqMSP products for the aforementioned samples were purified by Zymo Oligo Clean & Concentrator kit. One microliter of Purified DNA was quantified by Qubit. The rest of purified DNA was phosphorylated by T4 PNK, followed by adaptor ligation by adding adaptors and T4 DNA Ligase. The ligated products were purified by Zymo Oligo Clean & Concentrator kit, quantified by Qubit and analyzed by Agilent 2100 Bioanalyzer. The DNA libraries were mixed for NGS analysis.
(3) Sequence alignment and analysis.

### 2. Results

Analysis of sequencing data: We first tallied the sequencing depth (N) for each CpG site within the amplicons for the selected samples analyzed by the V1 and V2 assays. For CpG sites with no coverage, N was assigned a value of 1. The N values were log 2 transformed to obtain x (x=log₂ N). The depth of an amplicon (X) is calculated by averaging all x values for the CpG sites within the specified amplicon. X values are shown in the table below. Each X value represents the signal strength for the effective amplification of the specific biomarker in the mqMSP assay, which represents the DNA methylation level of that specific biomarker. The color gradient in the table below visually shows the X values with darker color represents stronger signal and higher DNA methylation level. Each row in the table represents a single amplicon in different samples while each column represents all amplicons in a single sample.

Further explanations on results for some representative samples:
(1) Analyses of biomarkers in the V1 assay: MBSR5, MBSR6, MBSR7 showed strong signals in most CRC samples, covering about 70% of CRC patients (assuming X values more than 14 as the threshold for preferred good detection). Other markers complemented these three markers for the patients not covered by MBSR5, MBSR6 or MBSR7. For example, MBSF10 complemented the three markers in CRC patients 22 and 26, while MBSF9 complemented the three markers in CRC patient 17. Additionally, overall, these biomarkers yielded significantly higher signals in CRC patients than in healthy individuals. These data further confirmed the principles and appropriateness for the biomarker combination in the V1 assay.

| | Sample type | | | | | | | | | | | | | |
|---|---|---|---|---|---|---|---|---|---|---|---|---|---|---|
| | CRC | CRC | CRC | CRC | CRC | CRC | CRC | CRC | CRC | CRC | CRC | CRC | CRC | |
| | Sample ID | | | | | | | | | | | | | |
| | 2 | 3 | 4 | 5 | 6 | 7 | 8 | 9 | 10 | 11 | 12 | 13 | 14 | |
| MBSR16 | 0.00 | 6.75 | 7.48 | 13.09 | 12.07 | 12.77 | 11.21 | 12.53 | 11.38 | 0.00 | 0.00 | 5.70 | 11.74 | |
| MBSF9 | 16.58 | 16.67 | 11.58 | 16.52 | 16.59 | 16.72 | 10.24 | 16.12 | 12.29 | 12.81 | 15.22 | 12.72 | 15.67 | |
| MBSR11 | 11.23 | 12.22 | 8.03 | 11.21 | 11.17 | 13.31 | 11.93 | 11.87 | 11.66 | 10.44 | 9.78 | 7.15 | 12.73 | |
| MBSF10 | 15.41 | 15.85 | 14.39 | 15.50 | 15.56 | 15.78 | 14.00 | 14.30 | 12.47 | 12.83 | 14.64 | 13.99 | 14.76 | |
| MBSR9 | 9.76 | 9.85 | 9.08 | 9.48 | 9.25 | 9.53 | 7.96 | 9.57 | 7.29 | 7.52 | 7.58 | 9.30 | 9.01 | |
| MBSR8 | 6.21 | 10.75 | 11.21 | 9.75 | 8.82 | 10.50 | 8.13 | 9.68 | 9.60 | 8.02 | 0.00 | 10.58 | 8.58 | |
| MBSR7 | 8.41 | 17.09 | 13.79 | 16.20 | 15.77 | 17.33 | 15.91 | 16.70 | 15.46 | 14.90 | 15.69 | 15.82 | 16.48 | |
| MBSR6 | 15.86 | 17.03 | 15.27 | 16.16 | 15.80 | 17.06 | 14.90 | 16.64 | 15.89 | 14.88 | 14.90 | 16.27 | 15.82 | |
| MBSF15 | 11.70 | 13.15 | 8.12 | 12.49 | 13.01 | 13.44 | 6.66 | 12.38 | 11.14 | 10.02 | 6.29 | 11.23 | 12.17 | |
| MBSR5 | 16.19 | 16.93 | 12.10 | 16.06 | 15.84 | 16.46 | 13.47 | 16.62 | 15.54 | 14.90 | 15.58 | 15.96 | 16.15 | |
| | Sample type | | | | | | | | | | | | | |
| | CRC | CRC | CRC | CRC | CRC | CRC | CRC | CRC | CRC | CRC | CRC | CRC | CRC | CRC |
| | Sample ID | | | | | | | | | | | | | |
| | 15 | 16 | 17 | 18 | 19 | 20 | 21 | 22 | 23 | 24 | 25 | 26 | 27 | 28 |
| MBSR16 | 0.00 | 0.00 | 5.86 | 0.00 | 12.76 | 6.21 | 6.69 | 6.38 | 6.60 | 0.00 | 10.32 | 6.17 | 5.64 | 0.00 |
| MBSF9 | 15.78 | 9.83 | 14.94 | 14.11 | 11.32 | 15.34 | 16.36 | 11.32 | 10.31 | 6.21 | 5.93 | 9.34 | 9.44 | 9.29 |
| MBSR11 | 12.14 | 12.15 | 7.62 | 12.13 | 12.79 | 13.31 | 9.05 | 8.11 | 8.88 | 11.68 | 11.64 | 7.76 | 9.14 | 11.19 |
| MBSF10 | 15.29 | 14.66 | 10.07 | 14.16 | 15.77 | 15.44 | 11.19 | 14.94 | 10.64 | 13.94 | 13.26 | 14.54 | 13.52 | 14.02 |
| MBSR9 | 8.29 | 8.12 | 0.00 | 6.66 | 9.81 | 8.34 | 6.27 | 0.00 | 8.43 | 6.60 | 8.32 | 5.67 | 8.30 | 0.00 |
| MBSR8 | 11.03 | 11.94 | 0.00 | 8.91 | 9.06 | 8.01 | 6.25 | 5.86 | 9.32 | 10.83 | 9.18 | 0.00 | 0.00 | 6.00 |
| MBSR7 | 16.83 | 17.50 | 11.82 | 15.61 | 17.31 | 17.01 | 12.86 | 12.55 | 16.81 | 14.50 | 14.84 | 12.30 | 14.43 | 15.55 |
| MBSR6 | 14.24 | 16.16 | 12.84 | 16.04 | 15.00 | 16.99 | 15.86 | 11.45 | 11.33 | 7.97 | 7.94 | 13.57 | 15.03 | 15.42 |
| MBSF15 | 11.70 | 11.44 | 6.51 | 7.40 | 7.63 | 12.24 | 12.11 | 6.93 | 11.30 | 0.00 | 0.00 | 9.93 | 6.43 | 6.86 |
| MBSR5 | 16.60 | 16.54 | 13.11 | 16.44 | 17.50 | 17.14 | 12.79 | 12.32 | 16.23 | 7.97 | 10.14 | 13.11 | 15.65 | 16.06 |

| | Sample type | | | | | | | | |
|---|---|---|---|---|---|---|---|---|---|
| | Healthy | Healthy | Healthy | Healthy | Healthy | Volunteer | Volunteer | Volunteer | Volunteer |
| | Sample ID | | | | | | | | |
| | 1 | 2 | 3 | 4 | 5 | 6 | 7 | 8 | 9 |
| MBSR16 | 5.81 | 0.00 | 0.00 | 6.11 | 6.39 | 0.00 | 0.00 | 0.00 | 0.00 |
| MBSF9 | 10.92 | 14.44 | 9.17 | 9.75 | 10.25 | 14.71 | 0.00 | 8.92 | 13.53 |
| MBSR11 | 6.43 | 13.29 | 7.30 | 9.94 | 11.13 | 0.00 | 9.68 | 7.23 | 12.09 |
| MBSF10 | 10.07 | 14.20 | 13.92 | 14.22 | 11.68 | 6.74 | 9.47 | 10.33 | 14.72 |
| MBSR9 | 0.00 | 0.00 | 6.64 | 8.11 | 9.15 | 8.29 | 0.00 | 0.00 | 0.00 |
| MBSR8 | 0.00 | 0.00 | 0.00 | 7.40 | 7.29 | 6.98 | 9.07 | 10.10 | 7.52 |
| MBSR7 | 15.93 | 16.62 | 12.31 | 15.34 | 16.27 | 15.93 | 13.90 | 14.50 | 15.28 |
| MBSR6 | 14.87 | 16.53 | 11.09 | 11.91 | 13.23 | 13.41 | 12.00 | 11.98 | 15.07 |
| MBSF15 | 10.52 | 0.00 | 6.23 | 6.87 | 7.44 | 0.00 | 0.00 | 5.93 | 6.39 |
| MBSR5 | 15.03 | 16.45 | 15.71 | 15.84 | 12.91 | 16.07 | 13.72 | 15.35 | 15.62 |

(2) Analyses of biomarkers in the V2 assay: QKI showed a strong signal in most CRC patients, covering about 90% of CRC patients (assuming X values more than 10 as the threshold for preferred good detection). Other biomarkers complemented QKI for the patients not covered by QKI. For example, NDRG4 provided the complementation for CRC patient 16. MBSF9, QKI and NDRG4 can be used as complementary markers for CRC patients 6, 13, 14, 15 and 16. These data further confirmed the principles and appropriateness for the biomarker combination in the V2 assay.

| | Sample type | | | | | | | | | |
|---|---|---|---|---|---|---|---|---|---|---|
| | CRC | CRC | CRC | CRC | CRC | CRC | CRC | CRC | CRC | CRC |
| | Sample ID | | | | | | | | | |
| | 1 | 2 | 3 | 4 | 5 | 6 | 7 | 8 | 9 | 10 |
| QKI | 15.43 | 17.67 | 16.55 | 16.50 | 10.09 | 10.23 | 10.30 | 18.04 | 16.56 | 17.17 |
| NDRG4 | 11.29 | 8.89 | 13.35 | 12.65 | 0.00 | 11.55 | 13.28 | 6.51 | 12.47 | 12.89 |
| MBSF8 | 6.63 | 11.68 | 10.61 | 8.86 | 11.02 | 0.00 | 9.54 | 7.30 | 6.13 | 8.85 |
| MBSR13 | 0.00 | 11.90 | 11.05 | 10.66 | 11.15 | 10.59 | 11.67 | 9.95 | 0.00 | 11.18 |
| MBSR16 | 0.00 | 8.41 | 0.00 | 0.00 | 0.00 | 0.00 | 0.00 | 6.25 | 6.00 | 6.13 |
| MBSF9 | 10.67 | 13.04 | 12.22 | 11.38 | 13.72 | 7.53 | 12.28 | 11.48 | 11.22 | 12.43 |

| | Sample type | | | | | | | | | |
|---|---|---|---|---|---|---|---|---|---|---|
| | CRC | CRC | CRC | CRC | CRC | CRC | CRC | CRC | CRC | Volunteer |
| | Sample ID | | | | | | | | | |
| | 11 | 12 | 13 | 14 | 15 | 16 | 17 | 18 | 19 | 1 |
| QKI | 13.07 | 15.24 | 15.95 | 11.32 | 15.48 | 6.54 | 18.19 | 17.32 | 17.14 | 16.27 |
| NDRG4 | 5.73 | 5.91 | 7.55 | 10.00 | 10.57 | 11.40 | 13.25 | 13.98 | 6.51 | 6.38 |
| MBSF8 | 9.70 | 0.00 | 6.98 | 0.00 | 7.67 | 0.00 | 12.81 | 11.12 | 11.34 | 0.00 |
| MBSR13 | 11.00 | 0.00 | 9.52 | 0.00 | 9.54 | 0.00 | 13.04 | 11.35 | 12.03 | 0.00 |
| MBSR16 | 6.46 | 0.00 | 0.00 | 0.00 | 0.00 | 0.00 | 9.63 | 6.66 | 7.79 | 0.00 |
| MBSF9 | 11.64 | 11.33 | 9.39 | 0.00 | 8.89 | 9.87 | 14.15 | 12.98 | 12.70 | 7.22 |

### Example 11

Methods with Double and Triple Fluorescence for Quantifying Multiple DNA Methylation Markers

### 1. Methods

(1) Double fluorescence mqMSP: The MGB probes for the multiple DNA methylation markers (MBSF9, MBSR16, MBSF8, MBSR13, NDRG4, QKI) were labeled with FAM fluorescence group. The MGB probe for the internal control assay was labeled with VIC fluorescence group. The sequences are summarized in the table of example 4. Methods for detection are described in example 7 (V2 assay).
(2) Triple fluorescence mqMSP: The DNA methylation markers were divided into two groups. FAM labeled MGB probes were used for group 1 including MBSF9, MBSR16, MBSF8, MBSR13, NDRG4, NPY and QKI, while VIC labeled MGB probes were used for group 2 including MBSF15, MBSR5, MBSR6, MBSR7, MBSR8 and MBSR9. The BHQ probe for the internal control assay was labeled with CY5 fluorescence group.

TABLE 5
Primers and probes for group 1 markers

| Name | Sequence (5′-3′) |
|---|---|
| MBSF9_F | TTCGTCGTTGTTTTTCGC (SEQ ID NO: 10) |
| MBSF9_R | GTTAACCGCGAAATCCG (SEQ ID NO: 11) |
| MBSF9-probe | 5′FAM-AACAACGAATCGCGC-3′MGB |
| | (SEQ ID NO: 12) |
| MBSF8_F | GTTTAGGGGTTTTTTCGGC (SEQ ID NO: 40) |
| MBSF8_R | CTAACTAAAACGCCGCG (SEQ ID NO: 41) |
| MBSF8-probe | 5′FAM-TAGTTTTGTATTGTAGGAGCGC-3′MGB |
| | (SEQ ID NO: 42 |
| MBSR13_F | GTTCGGGTTTTGCGC (SEQ ID NO: 43) |
| MBSR13_R | CTAAAAACTCCTCCGACG (SEQ ID NO: 44) |
| MBSR13-probe | 5′FAM-ACGCGAACGCGACGC-3′MGB |
| | (SEQ ID NO: 45) |
| MBSR16_F | GGGGTTCGAGTGGTC (SEQ ID NO: 37) |
| MBSR16_R | CGTCCCCTAAACGCG (SEQ ID NO: 38) |
| MBSR16-probe | 5′FAM-TATACGATCGGAGCGTTT-3′MGB |
| | (SEQ ID NO: 39) |
| NDRG4_F | CGGTTTTCGTTCGTTTTTTCG |
| | (SEQ ID NO: 46) |
| NDRG4_R | GTAACTTCCGCCTTCTACGC |
| | (SEQ ID NO: 47) |
| NDRG4_probe | 5′FAM-CTAAAATACCCGATAAAC-3′MGB |
| | (SEQ ID NO: 48) |
| NPY_F | GGAGTTATTTAAGCGTGATTGTTC |
| | (SEQ ID NO: 58) |
| NPY_R | AATAAAATACAAAAAACGAATCGCG |
| | (SEQ ID NO: 59) |
| NPY_probe | 5′FAM-AAACTTCCTCGCCGCGA-3′MGB |
| | (SEQ ID NO: 60) |
| QKI-F | TTTAGTTTCGGCGGTTATATTTTC |
| | (SEQ ID NO: 49) |
| QKI-R | CTACTCTCGAAAAAACTCGACG |
| | (SEQ ID NO: 50) |
| QKI_probe | 5′FAM-CACCGAATCCGCGCA-3′MGB |
| | (SEQ ID NO: 51) |

TABLE 6
Primers and probes for group 2 markers

| Name | Sequence (5′-3′) |
|---|---|
| MBSF15_F | TTTCGTTTGGATTCGGTAAC |
| | (SEQ ID NO: 16) |
| MBSF15_R | CCCGAACAAAACGCG (SEQ ID NO: 17) |
| MBSF15-probe | 5′VIC-TTTATTTTCGATTGAGTGGAT-3′MGB |
| | (SEQ ID NO: 18) |
| MBSR5_F | GTTTATTCGAGTAGGACGC (SEQ ID NO: 19) |
| MBSR5_R | CCGACAACGAAATAAAAAAATCG |
| | (SEQ ID NO: 20) |
| MBSR5-probe | 5′VIC-TTATTTAGTCGGAGGTGAGGA-3′MGB |
| | (SEQ ID NO: 21) |
| MBSR6_F | GGGTTTAAGCGGGGTTC (SEQ ID NO: 22) |
| MBSR6_R | ATTTCACTCTAAAAAATCCATCG |
| | (SEQ ID NO: 23) |
| MBSR6-probe | 5′VIC-TCCCCGACGACTCT-3′MGB |
| | (SEQ ID NO: 24) |
| MBSR7_F | GTTTCGAGGTAGTTTCGC (SEQ ID NO: 25) |
| MBSR7_R | TTATATTTACCAAACGACAACG |
| | (SEQ ID NO: 26) |
| MBSR7-probe | 5′VIC-CTCGAAAACTCGCGAAA-3′MGB |
| | (SEQ ID NO: 27) |
| MBSR8_F | AAATTTAGGCGGTAGTGC (SEQ ID NO: 28) |
| MBSR8_R | CCTATTAAAAACACCCGCG (SEQ ID NO: 29) |
| MBSR8-probe | 5′VIC-CTACGCGCCCTCACAA-3′MGB |
| | (SEQ ID NO: 30) |
| MBSR9_F | TTTTTTACGTAGGCGGC (SEQ ID NO: 31) |
| MBSR9_R | CCGCTAAAAACGCCG (SEQ ID NO: 32) |
| MBSR9-probe | 5′VIC-CCCGTACCCGCGCC-3′MGB |
| | (SEQ ID NO: 33) |

TABLE 7
Primers and probe for internal control gene
(underlined base represents the mutant base)

| Name | Sequence (5′-3′) |
|---|---|
| ACTB_F | GAGGGAGGAAGTTATGGTAGGTTAT (SEQ ID NO: 1) |
| ACTB_R | TCCTAACCACCTTCTCAACCTTAAA (SEQ ID NO: 2) |
| ACTB_probe | 5′CY5-AGAAGGTAGTTTGAAGTTGGT-3′BHQ |
| | (SEQ ID NO: 9) |

The reaction mixture contains the following:

| | Initial | Final | Volume |
|---|---|---|---|
| Component | concentration | concentration | (μL) |
| KAPA PROBE FAST | 2× | 1× | 12.5 |
| qPCR Master Mix | | | |
| Group 1 primer mix | 12.5 μM | 0.25 μM | 0.5 |
| Group 1 probe mix | 10 μM | 0.1 μM | 0.25 |
| Group 2 primer mix | 12.5 μM | 0.25 μM | 0.5 |
| Group 2 probe mix | 10 μM | 0.1 μM | 0.25 |
| ACTB primer mix | 5 μM | 0.05 μM | 0.25 |
| ACTB probe | 30 μM | 0.06 μM | 0.05 |
| ROX Low | 50× | 1× | 0.5 |
| H₂O | / | / | 0.2 |
| DNA template | / | / | 10 |
| Total volume | / | / | 25 |

### Thermocycling Condition on ABI 7500:

| Setting/Block | All channel |
|---|---|
| Polymerase activation | 95° C., 3 min |
| Denaturation | 95° C., 3 s |
| Annealing | 60° C., 30 s |
| Extension | 72° C., 30 s |
| GOTO 2 | 45 cycles |

(3) Sensitivity test for triple fluorescence mqMSP: 2%, 1%, 0.5% and 0.1% Bis-Meth DNA samples were prepared by mixing cell line DNA and buffy coat DNA. 10 ng/reaction was used to test the sensitivity of the triple fluorescence assay.
(4) cfDNA detection by triple fluorescence mqMSP: cfDNA samples were extracted from different sample types (CRC, benign polyps, advanced adenoma and healthy controls). 200 ng carrier DNA was added to each extracted cfDNA, followed by bisulfite conversion and triple fluorescence mqMSP detection.
(5) Comparison between double and triple fluorescence assays: 200 ng carrier DNA was added to each cfDNA sample from CRC tissues, followed by bisulfite conversion and double and triple fluorescence mqMSP detection.

### 2. Results

(1) The results for the sensitivity test for triple fluorescence mqMSP assay was summarized below: DNA methylation signals (FAM and VIC fluorescence signals) were detected for mimic samples with different methylation levels, up to 0.1% methylation. This demonstrates good sensitivity of the assay.

| | FAM | Average | Cq Std. | VIC | Average | Cq Std. | CY5 | Average | Cq Std. |
|---|---|---|---|---|---|---|---|---|---|
| Sample | Cq | FAM Cq | Dev | Cq | VIC Cq | Dev | Cq | CY5 Cq | Dev |
| 2% Meth DNA | 35.84 | 36.06 | 0.32 | 37.62 | 36.63 | 1.41 | 42.89 | 42.89 | N/A |
| 2% Meth DNA | 36.29 | | | 35.63 | | | N/A | | |
| 1% Meth DNA | 38.26 | 38.51 | 0.35 | 37.72 | 37.86 | 0.20 | 43.55 | 43.55 | N/A |
| 1% Meth DNA | 38.76 | | | 38.00 | | | N/A | | |
| 0.5% Meth DNA | 38.05 | 38.90 | 1.20 | 38.29 | 37.95 | 0.49 | 42.97 | 42.32 | 0.91 |
| 0.5% Meth DNA | 39.75 | | | 37.60 | | | 41.68 | | |
| 0.1% Meth DNA | 40.33 | 38.97 | 1.92 | N/A | 42.45 | N/A | 42.56 | 42.41 | 0.21 |
| 0.1% Meth DNA | 37.62 | | | 42.45 | | | 42.27 | | |

(2) Results for triple fluorescence mqMSP for cfDNA samples from different sample types: DNA methylation signals (FAM and VIC fluorescence signals) were detected for cfDNA from CRC, while cfDNA from healthy control samples showed very low methylation levels with almost non-detectable signal.

| | | | | | FAM | | VIC | | CY5 |
|---|---|---|---|---|---|---|---|---|---|
| | Plasma | | cfDNA | Average | Cq | | Cq | Average | Cq |
| | DNA | Clinical | amount | FAM | Std. | Average | Std. | CY5 | Std. |
| Sample type | ID | stage | (ng) | Cq | Dev | VIC Cq | Dev | Cq | Dev |
| CRC | 869R | 3 | 23.88 | 33.23 | 0.01 | 33.46 | 0.42 | 36.41 | 0.63 |
| CRC | 891R | 4 | 27.81 | 26.12 | 0.06 | 26.50 | 0.03 | N/A | N/A |
| CRC | 168R | 4 | 21.32 | 36.91 | 0.50 | 37.32 | 1.19 | 37.17 | 0.01 |
| CRC | 584R | 4 | 52.61 | 26.16 | 0.18 | 26.68 | 0.05 | 43.64 | 0.65 |
| CRC | 559R | 4 | 50.61 | 25.12 | 0.06 | 24.82 | 0.02 | N/A | N/A |
| Benign polyp | 420Y | / | 50.48 | N/A | N/A | N/A | N/A | 42.17 | 3.28 |
| Advanced | 224Y | / | 38.91 | N/A | N/A | N/A | N/A | IN/A | N/A |
| adenoma | | | | | | | | | |
| Healthy | 662Y | / | 32.07 | N/A | N/A | N/A | N/A | 38.58 | 0.86 |
| control | | | | | | | | | |
| Healthy | 661Y | / | 18.00 | N/A | N/A | N/A | N/A | 40.08 | 0.92 |
| control | | | | | | | | | |
| Healthy | 698Y | / | 20.00 | N/A | N/A | 36.22 | N/A | 38.31 | 0.77 |
| control | | | | | | | | | |

(3) Results for comparing the double and triple fluorescence mqMSP assays:

As shown in FIG. 8, for double fluorescence mqMSP, FAM signal (target1) represents the DNA methylation levels of the markers while VIC signal (target2) represents the internal control to reflect the input DNA amount.

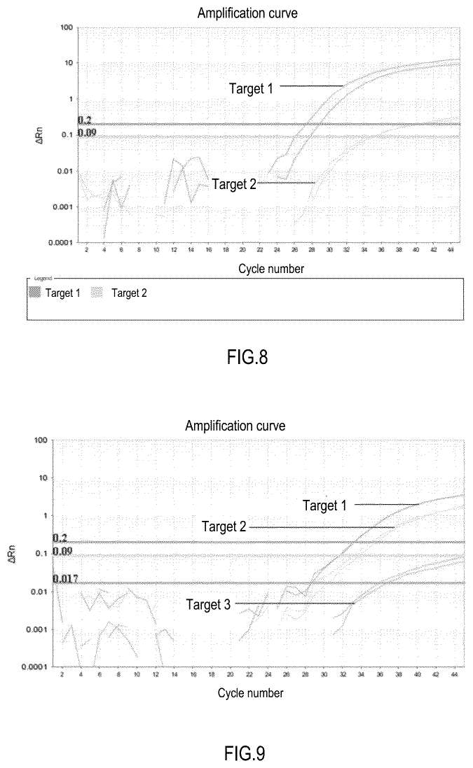

In FIG. 9, for triple fluorescence mqMSP, FAM (target1) and VIC (target2) represent the DNA methylation levels of the two groups of markers, while CY5 (target3) represents the internal control to reflect the input DNA amount.

Results showed both methods can detect the DNA methylation levels of the CRC cfDNA samples.

### Example 12

### Multiple DNA Methylation Markers Quantified by MALDI-TOF Mass Spectrometry

In this example, DNA methylation quantification was achieved by combining methylation-sensitive restriction enzymes, real-competitive PCR, single base extension and MALDI-TOF mass spectrometry.

### 1. Methods

(1) DNA samples were extracted from one CRC tumor tissue, one normal tissue and one buffy coat. One microliter of each extracted DNA was quantified by Thermo Qubit® dsDNA HS kit.
(2) One microgram of DNA from the above samples were fragmented into smaller sizes (about 170 bp) using the Bioruptor sonicator with the following setting:

| Cycling condition (on/off time) | Cycle number |
|---|---|
| 30″/30″ | 13 |

(3) Purification of fragmented DNA: Zymo Oligo Clean & Concentrator kit was used for DNA purification after sonication to remove very small fragments and concentrate the DNA. Each DNA was eluted in 32 μL. Multiple fragmented DNA from the same original sample were pooled into a single tube and quantified by Qubit by taking 1 μL of DNA.
(4) Enzyme digestion: Four methylation sensitive restriction enzymes including HpaII, HhaI, AciI and BstUI (all from New England Biolabs) were used to digest the DNA samples in a 50 μL digestion system with 20 U of each enzyme and 100 ng fragmented DNA. HpaII, HhaI and AciI were added first to perform the digestion at 37° C. for 16 hours, followed by adding 2 μL BstUI to further digest at 60° C. for 6 hours.

The digestion system is:

| Component | Volume |
|---|---|
| H₂O | 31.6 μL |
| 10X Cutsmart buffer | 5 μL |
| Hpa II (50 U/μL) | 0.4 μL |
| Hha I (20 U/μL) | 1 μL |
| Aci I (10 U/μL) | 2 μL |
| DNA (10 ng/μL) | 10 μL |
| Total Volume | 50 μL |
| Reaction condition | 37° C. for 16 h, followed by adding |
| | BstUI and incubate at 60° C. for 6 h |

### Mock Control System (Without Adding Enzyme):

| Component | Volume |
|---|---|
| H₂O | 35 μL |
| 10X Cutsmart buffer | 5 μL |
| DNA (10 ng/μL) | 10 μL |
| Total volume | 50 μL |
| Reaction condition | 37° C. for 16 h, followed by 60° C. for 6 h |

(5) Purification of digestion product: Zymo Oligo Clean & Concentrator kit was used for purification of the 50 μL enzyme digestion product to remove impurities and concentrate DNA. DNA was eluted in a final volume of 12 μL. One microliter was used for quantification by Qubit and the remaining DNA was used for subsequent detection.
(6) Real-competitive PCR analysis:

Based on the RRBS results, a total of 14 genomic regions including FGF12, ELOVL2 and HSPA1A were shown to have significantly higher DNA methylation levels in CRC tumor tissues than in other tissue samples and buffy coat samples, and thus may serve as tumor-specific DNA methylation markers. As such, PCR primers and extension primers were designed for multiple regions within the genes including FGF12, ELOVL2 and HSPA1A, following the principle that each amplicon must contain at least three cutting sites for the aforementioned methylation sensitive restriction enzymes. An internal control assay targeting the ACTB gene with almost no methylation in all samples involved was used for quality control.

TABLE 8
Biomarker information

| Gene | Biomarker name | Chromosome location |
|---|---|---|
| ACTB | QC | chr7: 5530563-5530637 |
| FGF12 | RRB10 | chr3: 192408801-192408861 |
| ELOVL2 | RRB13 | chr6: 11044173-11044248 |
| HSPA1A | RRB14 | chr6: 31815656-31815713 |
| ELOVL2/ELOVL2-AS1 | RRB16 | chr6: 11043728-11043798 |
| SYNE1 | RRB17_1 | chr6: 152636778-152636838 |
| SYNE1 | RRB17_2 | chr6: 152636910-152636974 |
| SFMBT2 | RRB20 | chr10: 7409123-7409201 |
| FLI1/SENCR | RRB21_4 | chr11: 128693445-128693513 |
| FBN1 | RRB26_2 | chr15: 48645387-48645456 |
| / | RRB2 | chr1: 34930091-34930158 |
| AL096828.1 | RRB30 | chr20: 63179214-63179293 |
| LONRF2 | RRB6_1 | chr2: 100322008-100322068 |
| LONRF2 | RRB6_4 | chr2: 100322347-100322411 |
| LONRF2 | RRB6_5 | chr2: 100322420-100322494 |

### A. PCR Primers Designed for the DNA Methylation Markers:

TABLE 9
PCR primers used in the experiments

| Primer | |
|---|---|
| name | Sequence (5′ to 3′) |
| RRB6_5-F | ACGTTGGATGGCTGCGGTAACGCAGTGAC |
| | (SEQ ID NO: 61) |
| RRB6_5-R | ACGTTGGATGCAGGAGCCCTCAGGCGAGT |
| | (SEQ ID NO: 62) |
| RRB20-F | ACGTTGGATGAAGCTTCGGTCCCGGTCC |
| | (SEQ ID NO: 63) |
| RRB20-R | ACGTTGGATGCGCGCTGCACTTAATAGTGG |
| | (SEQ ID NO: 64) |
| RRB6_4-F | ACGTTGGATGACCGCGGGCGCAATCTGAG |
| | (SEQ ID NO: 65) |
| RRB6_4-R | ACGTTGGATGTTACCTAAGCCGGCGGGACT |
| | (SEQ ID NO: 66) |
| RRB21_4-F | ACGTTGGATGTATGGAGGGAGAGTTGGCTA |
| | (SEQ ID NO: 67) |
| RRB21_4-R | ACGTTGGATGTTTTCGTCCGAGTCTTCCCC |
| | (SEQ ID NO: 68) |
| RRB16-F | ACGTTGGATGTTAGGGCCCGAACCCCAGAA |
| | (SEQ ID NO: 69) |
| RRB16-R | ACGTTGGATGTGGAGCGGAGAACGGCGTTT |
| | (SEQ ID NO: 70) |
| RRB10-F | ACGTTGGATGAGAGAAGGAGAGGAAGGCAG |
| | (SEQ ID NO: 71) |
| RRB10-R | ACGTTGGATGCTCCTAATCTTCAGAACCGC |
| | (SEQ ID NO: 72) |
| QC-F | ACGTTGGATGGAGGCTCTGTGCTCGCGGG |
| | (SEQ ID NO: 73) |
| QC-R | ACGTTGGATGGGCGCCCTATAAAACCCAG |
| | (SEQ ID NO: 74) |
| RRB2-F | ACGTTGGATGCCCGTCCGCTTAGGAGACT |
| | (SEQ ID NO: 75) |
| RRB2-R | ACGTTGGATGCCAAGCCAGGAGCTGAGGT |
| | (SEQ ID NO: 76) |
| RRB17_2-F | ACGTTGGATGTGCTCAGCCGCGCTCCCC |
| | (SEQ ID NO: 77) |
| RRB17_2-R | ACGTTGGATGCTCACCCCCCAGCGAGCAG |
| | (SEQ ID NO: 78) |
| RRB14-F | ACGTTGGATGAGTTTCCGGCGTCCGGAAG |
| | (SEQ ID NO: 79) |
| RRB14-R | ACGTTGGATGCTGGAAACGGAACACTGGAT |
| | (SEQ ID NO: 80) |
| RRB13-F | ACGTTGGATGCCACAGCCGCTGCGGATCA |
| | (SEQ ID NO: 81) |
| RRB13-R | ACGTTGGATGTCCAGGAGAGAAAGAAAGCG |
| | (SEQ ID NO: 82) |
| RRB30-F | ACGTTGGATGAGGACCAGGCTTGCATGGG |
| | (SEQ ID NO: 83) |
| RRB30-R | ACGTTGGATGATGCGGTCCGGCGTGTCCA |
| | (SEQ ID NO: 84) |
| RRB26_2-F | ACGTTGGATGACCAAGAGCCCCGGGCCAG |
| | (SEQ ID NO: 85) |
| RRB26_2-R | ACGTTGGATGTGTCCCGGAACGTCCACAG |
| | (SEQ ID NO: 86) |
| RRB17_1-F | ACGTTGGATGTCTGCAGGACTGCGGGAGC |
| | (SEQ ID NO: 87) |
| RRB17_1-R | ACGTTGGATGCAGCGGGCTGAGATTGTTG |
| | (SEQ ID NO: 88) |
| RRB6_1-F | ACGTTGGATGTCCTCTAAGCGCTGGGCGAT |
| | (SEQ ID NO: 89) |
| RRB6_1-R | ACGTTGGATGCGCCGCCGCCCCAGTGTC |
| | (SEQ ID NO: 90) |

TABLE 10
Extension primers used in the experiments

| Primer name | Sequence (5′ to 3′) |
|---|---|
| RRB6_5-U | GCTGCTCTTGCGATG (SEQ ID NO: 91) |
| RRB20-U | CGGCGTGGAGGAAAG (SEQ ID NO: 92) |
| RRB6_4-U | TCTGAGCCCCTGCCCA (SEQ ID NO: 93) |
| RRB21_4-U | GGCGGCTGGTAACCCA (SEQ ID NO: 94) |
| RRB16-U | CCCCAGAACTCCCGAGG (SEQ ID NO: 95) |
| RRB10-U | GGAAGGCAGCAATTTAA (SEQ ID NO: 96) |
| QC-U | gGGCTGGGGTGGCGCGT (SEQ ID NO: 97) |
| RRB2-U | GCTTAGGGAACTCTCCTT (SEQ ID NO: 98) |
| RRB17_2-U | aGCCCCCTGCCCTCCGCGA (SEQ ID NO: 99) |
| RRB14-U | gtccAAGGACCGAGCTCTT (SEQ ID NO: 100) |
| RRB13-U | aCGCTGCGGATCATGGTGA (SEQ ID NO: 101) |
| RRB30-U | CCCTCCGCCCAGGGTCCAAA (SEQ ID NO: 102) |
| RRB26_2-U | ggtcaGGGCCAGGAAGCTGT (SEQ ID NO: 103) |
| RRB17_1-U | CCTGCCAAGCCGCCCTGGTGA (SEQ ID NO: 104) |
| RRB6_1-U | gggccCGGCTCCGCGCGGTCG (SEQ ID NO: 105) |

Competitors were designed by introducing single base mutations next to the 3′ of the extension primers based on the sequences of the amplicons. In the following table, the introduced mutation was underlined.

TABLE 11
Competitor sequences

| Name | Sequence (5′ to 3′) |
|---|---|
| RRB6_5_C | GCTGCGGTAACGCAGTGACCGCGCTCCAGGTCCGCGTCTC |
| | TTGCGATGGTTCCCCCACTCGCCTGAGGGCTCCTGC |
| | (SEQ ID NO: 106) |
| RRB20_C | AAGCTTCGGTCCCGGTCCCCCACCTCCCACCCCAGAGCGG |
| | CGTGGAGGAAAGGAGGAGCCCACTATTAAGTGCAGCGCGC |
| | (SEQ ID NO: 107) |
| RRB6_4_C | ACCGCGGGCGCAATCTGAGCCCCTGCCCAGGCGCAGCGGC |
| | CTCTCAGTCCCGCCGGCTTAGGTAAC |
| | (SEQ ID NO: 108) |
| RRB21_4_C | GTTTTCGTCCGAGTCTTCCCCGGCAGTAGGCGGTGGGTTA |
| | CCCGCAGCCCTAGCCAACTCTCCCTCCATAC |
| | (SEQ ID NO: 109) |
| RRB16_C | GTTAGGGCCCGAACCCCAGAACTCCCGAGGGAGGGTGTGT |
| | GCCGCGCGCGGGAAACGCCGTTCTCCGCTCCA |
| | (SEQ ID NO: 110) |
| RRB10_C | GAGAGAAGGAGAGGAAGGCAGCAATTTAAGTCCCTGCGGC |
| | CCGCGGTTCTGAAGATTAGGAG (SEQ ID NO: 111) |
| QC_C | GAGGCTCTGTGCTCGCGGGGCGGACGCGGTCTCGGCGGTG |
| | GTGGCGCGTGGCGCCGCTGGGTTTTATAGGGCGCC |
| | (SEQ ID NO: 112) |
| RRB2_C | GCCCGTCCGCTTAGGAGACTCTCCTTGGGGCTTTCCCGGT |
| | CGGCCGCCTGACCTCAGCTCCTGGCTTGG |
| | (SEQ ID NO: 113) |
| RRB17_2_C | TGCTCAGCCGCGCTCCCCGCCCTCCGCGAGCGGTTCCTCC |
| | TCCCGGCTGCTCGCTGGGGGGTGAG |
| | (SEQ ID NO: 114) |
| RRB14_C | GAGTTTCCGGCGTCCGGAAGGACCGAGCTCTTGTCGCGGA |
| | TCCAGTGTTCCGTTTCCAGC (SEQ ID NO: 115) |
| RRB13_C | GTCCAGGAGAGAAAGAAAGCGCGGCGGTGTCGGTGGCGGC |
| | GCGCGGCCCCAGTCACCATGATCCGCAGCGGCTGTGGC |
| | (SEQ ID NO: 116) |
| RRB30_C | AGGACCAGGCTTGCATGGGGACGGGGGTCTGCGTCCGCGT |
| | CCAGGGTCCAAAGGAGGGCGGTGGACACGCCGGACCGCAT |
| | C (SEQ ID NO: 117) |
| RRB26_2_C | ACCAAGAGCCCCGGGCCAGGAAGCTGTGAGGCAGGGGAGG |
| | GCCAGCGCCAGCTGTGGACGTTCCGGGACA |
| | (SEQ ID NO: 118) |
| RRB17_1_C | GTCTGCAGGACTGCGGGAGCCCAGCCGCCCTGGTGAGAGA |
| | GGACAACAATCTCAGCCCGCTGC (SEQ ID NO: 119) |
| RRB6_1_C | TCCTCTAAGCGCTGGGCGATCGGCTCCGCGCGGTCGGAGC |
| | CAGGACACTGGGGCGGCGGCG (SEQ ID NO: 120) |

### B. PCR Primer Mixture (1 μM for Each Primer):

PCR primer mixture

| Component | Starting concentration | Volume | Final concentration |
|---|---|---|---|
| RRB6_5 | 100 μM each | 2 μL each | 1 μM each |
| RRB20 | 100 μM each | 2 μL each | 1 μM each |
| RRB6_4 | 100 μM each | 2 μL each | 1 μM each |
| RRB21_4 | 100 μM each | 2 μL each | 1 μM each |
| RRB16 | 100 μM each | 2 μL each | 1 μM each |
| RRB10 | 100 μM each | 2 μL each | 1 μM each |
| QC | 100 μM each | 2 μL each | 1 μM each |
| RRB2 | 100 μM each | 2 μL each | 1 μM each |
| RRB17_2 | 100 μM each | 2 μL each | 1 μM each |
| RRB14 | 100 μM each | 2 μL each | 1 μM each |
| RRB13 | 100 μM each | 2 μL each | 1 μM each |
| RRB30 | 100 μM each | 2 μL each | 1 μM each |
| RRB26_2 | 100 μM each | 2 μL each | 1 μM each |
| RRB17_1 | 100 μM each | 2 μL each | 1 μM each |
| RRB6_1 | 100 μM each | 2 μL each | 1 μM each |
| H₂O | / | 140 μL | / |

| | |
|---|---|
| Final volume | 200 μL |

The above primer mixture was further diluted by adding 50 μL ddH₂O to 50 μL of the above mixture to obtain a working mixture concentration of 0.5 μM for each primer.

Preparation of competitors: Each competitor in powder form was dissolved in a final concentration of 1 μM. The concentrations were further determined by the Thermo Qubit® ssDNA kit. The actual concentrations in copy numbers were calculated by taking into account of the molecular weights of the competitors. The competitors were further diluted and mixed so that the amounts of the competitors added to the subsequent PCR reactions satisfied the following:

### Mock Digestion Samples (Tumor Tissue DNA/Normal Tissue DNA/Buffy Coat DNA):

| | Competitors added to samples without digestion |
|---|---|
| Each target | 6600 copies/PCR reaction |
| QC | 3300 copies/PCR reaction |

### Digestion Samples (Normal Tissue DNA/Buffy Coat DNA)

| N/BC | Competitors added to samples with digestion |
|---|---|
| Each target | 66 copies/PCR reaction |
| QC | 33 copies/PCR reaction |

### Digestion Samples T (Tumor Tissue DNA)

| T | Competitors added to samples with digestion |
|---|---|
| Each target | 3300 copies/PCR reaction |
| QC | 33 copies/PCR reaction |

### Mixture for Extension Primers:

Each unextended extension primer (UEP) in powder form was first dissolved to a final concentration of 200 μM, and mixed according to the following:

| | Starting | UEP | Volume |
|---|---|---|---|
| Assay | concentration [μM] | Molecular weight | added [μl] |
| RRB6_5-U | 200 | 4575 | 1.15 |
| RRB20-U | 200 | 4707.1 | 1.87 |
| RRB6_4-U | 200 | 4778.1 | 1.87 |
| RRB21_4-U | 200 | 4907.2 | 1.87 |
| RRB16-U | 200 | 5125.3 | 1.87 |
| RRB10-U | 200 | 5267.5 | 2.37 |
| QC-U | 200 | 5339.4 | 2.37 |
| RRB2-U | 200 | 5465.6 | 1.92 |
| RRB17_2-U | 200 | 5670.7 | 2.01 |
| RRB14-U | 200 | 5788.8 | 2.01 |
| RRB13-U | 200 | 5868.8 | 2.08 |
| RRB30-U | 200 | 6007.9 | 1.75 |
| RRB26_2-U | 200 | 6223 | 2.87 |
| RRB17_1-U | 200 | 6368.1 | 2.87 |
| RRB6_1-U | 200 | 6441.1 | 3.11 |
| | H₂O | | 18.01 |
| | | Total | 50 |

C. PCR Reactions: PCR System for Purified DNA After Either Enzymatic Digestion or Mock Digestion, Together with Competitors:

| | Final | | Volume per |
|---|---|---|---|
| Regents | concentration | | reaction [μL] |
| Water, HPLC grade | NA | | 7.8 |
| 10 × PCR buffer with 20 mM MgCl2 | 2 | mM MgCl2 | 2.5 |
| 25 mM dNTP Mix | 500 | μM | 0.5 |
| 25 mM MgCl2 | 2 | mM | 2 |
| 0.5 μM primer mixture | 0.1 | μM | 5 |
| 5 U/μl PCR polymerase | 1 | U/rxn | 0.2 |

| | | |
|---|---|---|
| (Mock) Digestion product (4 ng/μL) | / | 5 |
| Competitor mix | / | 2 |
| Total volume [μL] | / | 25 |

The samples were loaded into each reaction.

PCR thermocycling condition was set as the following:

| | | |
|---|---|---|
| 95° C. | 2 min | |
| 95° C. | 30 sec | 45 cycles |
| 56° C. | 30 sec | |
| 72° C. | 1 min | |
| 72° C. | 5 min | |
| 4° C. | Keep | |

### D. Take 5 μL of PCR Product and Add 2 μL of Shrimp Alkaline Phosphatase (SAP) for SAP Reaction, Using the Reaction System as the Following:

| | Final | Volume for |
|---|---|---|
| Reagent | concentration (in 7 μl) | each reaction [μL] |
| Ultrapure water treated with | NA | 1.53 |
| high-pressure steam | | |
| SAP buffer (10X) | 0.243x | 0.17 |
| SAP (1.7 U/μL) | 0.51 U/rnx | 0.3 |
| Total volume [μL] | / | 2 |

### Reaction Condition:

| | | |
|---|---|---|
| 37° C. | 40 min | |
| 85° C. | 5 min | 1 cycle |
| 4° C. | keep | |

### E. Extension Reaction: Two μL of Extension Mixture (See Table Below) was Added to the 7 μL SAP Reaction Product.

| | Final concentration | Volume for |
|---|---|---|
| Reagent | (in 9 μL) | each reaction [μL] |
| Ultrapure water | NA | 0.62 |
| IPLEX buffer | 0.222X | 0.2 |
| IPLEX terminator mix | 0.222X | 0.2 |
| Mixture of extension | 0.4-1.95 μM | 0.94 |
| primers | | |
| IPLEX enzyme | 0.142 U/μL | 0.04 |
| Total volume [μL] | | 2 |

### Reaction Condition:

| | | | |
|---|---|---|---|
| 95° C. | 30 sec | | |
| 95° C. | 5 sec | | 40 cycles |
| 54° C. | 5 sec | 5 cycles | |
| 80° C. | 5 sec | | |
| 72° C. | 3 min | | |
| 4° C. | Keep | | |

### F. Spotting of Samples and Analysis by MALDI-TOF Mass Spectrometry to Obtain Data.

### G. Analysis Algorithm.

The peak signals for the targets and their respective competitors were collected and their ratios were calculated. “call” represents a peak signal was produced from the primer extension, “0” represents peak signal of 0 and no primer extension occurred. The analysis was performed according to the following algorithm:

The copy numbers of the competitors added to each reaction were the following:

For PCR reactions with samples with no digestion (20 ng of either tumor tissue DNA, normal tissue DNA or buffy coat DNA):

| | Competitors added |
|---|---|
| Each target | 6600 copies /PCR reaction |
| QC | 3300 copies/PCR reaction |

For PCR Reactions with Samples with Digestion (20 ng of Either Normal Tissue Coat or Buffy Coat DNA):

| N/BC | Competitors added |
|---|---|
| Each target | 66 copies/PCR reaction |
| QC | 33 copies/PCR reaction |

For PCR Reactions with Samples with Digestion (20 ng of Tumor Tissue DNA):

| T | Competitors added |
|---|---|
| Each target | 3300 copies/PCR reaction |
| QC | 33 copies/PCR reaction |

Interpretation of Results for Samples with No Digestion

| Target DNA | Competitor | Target/ | |
|---|---|---|---|
| AvgFreq | AvgFreq | Competitor | Result |
| 0 | 0 | NA | Quality control failed |
| 1 | 0 | NA | Quality control failed |
| 0 | 1 | 0 | Quality control failed |
| Call 1 | Call 2 | Call 1/ | Quality control passed; |
| | | Call 2 | Target gene copy number = |
| | | | copy number of competitor × |
| | | | (Call 1/Call 2) |

Interpretation of Results for Samples (Normal Tissue DNA and Buffy Coat DNA) with Enzymatic Digestion:

| Target DNA | Competitor | Target/ | |
|---|---|---|---|
| AvgFreq | AvgFreq | Competitor | Result |
| 0 | 0 | NA | Quality control failed |
| 1 | 0 | NA | Quality control passed; |
| | | | Target gene copy number > (copy |
| | | | number of competitor) × 20 |
| 0 | 1 | 0 | Quality control passed; |
| | | | Target gene copy number < (copy |
| | | | number of competitor) × 1/21 |
| Call 1 | Call 2 | Call 1/ | Quality control passed; |
| | | Call 2 | Target gene copy number = copy |
| | | | number of competitor × (Call 1/ |
| | | | Call 2) |

Interpretation of Results for Samples (Tumor Tissue DNA) with Enzymatic Digestion:

| Target DNA | Competitor | Target/ | |
|---|---|---|---|
| AvgFreq | AvgFreq | Competitor | Result |
| 0 | 0 | NA | Quality control failed |
| 1 | 0 | NA | Quality control failed |
| 0 | 1 | 0 | Quality control passed; |
| | | | Target gene copy number < (copy |
| | | | number of competitor) × 1/21 |
| Call 1 | Call 2 | Call 1/ | Quality control passed; |
| | | Call 2 | Target gene copy number = copy |
| | | | number of competitor × (Call 1/ |
| | | | Call 2) |

(7) Plasma cfDNA detection using the methods with MALDI-TOF mass spectrometry as described above: We selected a total of 28 plasma cfDNA samples (15 CRC, 4 advanced adenoma, 4 polyps, and 5 healthy controls) for quantification of candidate DNA methylation markers using the methods described above.

### 2. Results

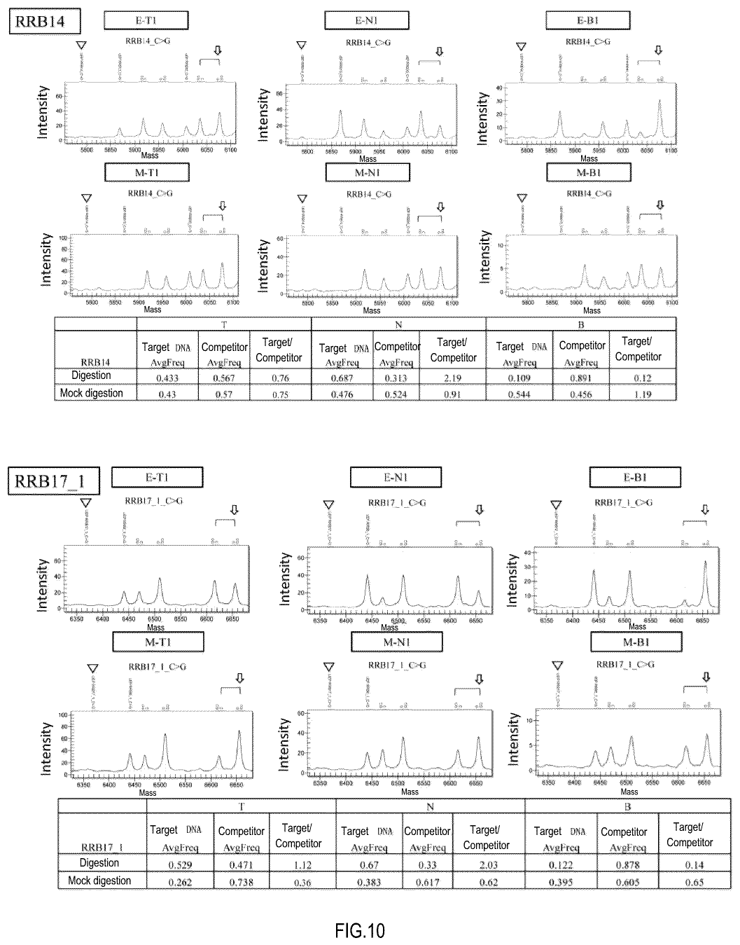

(1) As shown in FIG. 10 (top half), the candidate methylation marker RRB14 was analyzed in tumor tissue DNA, normal tissue DNA and buffy coat DNA with or without enzymatic digestion. In the three mock digestions (M-T1, M-N1 and M-B1), the mass spectrometry peak intensity ratios for the competitor and the target gene were close to 1:1, suggesting good PCR efficiency and similar input copy numbers for undigested DNA and competitor. In the three digestions (E-T1, E-N1 and E-B1), the mass spectrometry peak intensity ratios for the competitor and the target gene were different, with a ratio of 0.76 for tumor DNA E-T1, 2.19 for normal tissue DNA E-N1 and 0.12 for buffy coat DNA E-B1. Accordingly, the calculated methylated target copy numbers were 2508, 144.54 and 7.92 for tumor DNA, normal tissue DNA and buffy coat DNA, respectively, suggesting a substantially higher methylation level in the CRC tumor sample than in the other two samples.

Similarly, in FIG. 10 (bottom half), the methylation marker RRB17_1 was analyzed to have methylated target copy numbers of 3696, 133.98 and 9.24 for tumor DNA, normal tissue DNA and buffy coat DNA, respectively.

Copy numbers of all markers quantified as above are summarized below (copies):

| Correspond- | | | | | | | |
|---|---|---|---|---|---|---|---|
| ing | Marker | | | | | | |
| gene | name | E-T1 | E-N1 | E-B1 | M-T1 | M-N1 | M-B1 |
| FGF12 | RRB10 | 8283 | 324.72 | 357.06 | 8382 | 8910 | 8646 |
| ELOVL2 | RRB13 | 2706 | 27.06 | 0 | 4356 | 8580 | 7458 |
| HSPA1A | RRB14 | 2508 | 144.54 | 7.92 | 4950 | 6006 | 7854 |
| ELOVL2 | RRB16 | 4851 | 84.48 | 35.64 | 5280 | 6996 | 6732 |
| SYNE1 | RRB17_1 | 3696 | 133.98 | 9.24 | 2376 | 4092 | 4290 |
| SYNE1 | RRB17_2 | 4785 | 196.02 | 17.82 | 9240 | 12804 | 13332 |
| SFMBT2 | RRB20 | 6567 | 239.58 | 73.26 | 8052 | 9372 | 10956 |
| FBN1 | RRB26_2 | 1551 | 18.48 | 17.82 | 2640 | 4224 | 4224 |
| AL096828.1 | RRB30 | 2046 | 126.06 | 0 | 2244 | 3168 | 3300 |
| LONRF2 | RRB6_4 | 5907 | 1.98 | 5.28 | 7326 | 10494 | 12276 |

(2) Results for plasma cfDNA (copies):
   CRC Plasma cfDNA Results:

| | Sample type | | | | | | | |
|---|---|---|---|---|---|---|---|---|
| | CRC | CRC | CRC | CRC | CRC | CRC | CRC | CRC |
| | Tumor stage | | | | | | | |
| | 1 | 1 | 1 | 1 | 2 | 2 | 2 | 2 |
| | Assay | | | | | | | |
| | 2463RP | 1451RP | 1501RP | 2064RP | 3569RP | 2477RP | 2932RP | zy1-238 |
| RRB10 | 111.21 | 99 | 69.63 | 17.16 | 41.25 | 119.13 | 181.17 | 112.53 |
| RRB13 | 0 | 0 | 0 | 0 | 2.97 | 0 | 0 | 0 |
| RRB14 | 0 | 4.62 | 0 | 0 | 0 | 5.28 | 2.31 | 5.61 |
| RRB16 | 0 | 6.27 | 3.3 | 11.55 | 0 | 76.89 | >660 | 31.35 |
| RRB17_1 | 4.62 | 0 | 0 | 0 | 0 | 7.26 | 0 | 11.55 |
| RRB17_2 | 0 | 0 | 6.27 | 0 | 0 | 35.31 | 18.48 | 17.49 |
| RRB20 | 12.87 | 57.75 | 9.57 | 0 | 15.84 | 10.89 | 27.06 | 13.2 |
| RRB21_4 | 0 | 15.18 | 3.3 | 0 | 14.52 | 2.64 | 7.59 | 0 |
| RRB26-2 | 0 | 0 | 0 | 0 | 0 | 0 | 0 | 2.64 |
| RRB30 | 0 | 0 | 0 | 0 | 0 | 7.26 | 3.3 | 0 |
| RRB6_4 | 0 | 0 | 0 | 0 | 0 | 0 | 0 | 10.23 |

| | Sample type | | | | | | |
|---|---|---|---|---|---|---|---|
| | CRC | CRC | CRC | CRC | CRC | CRC | CRC |
| | Tumor stage | | | | | | |
| | 3 | 3 | 3 | 3 | 3 | 4 | 4 |
| | Assay | | | | | | |
| | sfg-8-0 | 1722RP | 1834RP | 1816RP | 869R | 584R | 559R |
| RRB10 | 165.66 | 62.7 | 254.1 | 80.85 | 158.86 | 578.11 | 499.26 |
| RRB13 | 0 | 0 | 0 | 5.61 | 61.02 | >660 | >660 |
| RRB14 | 6.6 | 0 | 1.98 | 2.6 | 318.06 | 8.25 | >660 |
| RRB16 | 8.91 | 1.65 | 28.71 | 13.86 | 125.65 | >660 | >660 |
| RRB17_1 | 4.29 | 16.5 | 10.56 | 4.29 | 44.1 | 452.29 | 578.11 |
| RRB17_2 | 13.2 | 9.24 | 14.85 | 0 | >660 | >660 | >660 |
| RRB20 | 17.82 | 55.77 | 5.61 | 49.83 | 168.22 | 4681.29 | >660 |
| RRB21_4 | 17.16 | 0 | 4.95 | 128.04 | 224.81 | >660 | >660 |
| RRB26-2 | 0 | 0 | 0 | 0 | 22.28 | 1908.18 | >660 |
| RRB30 | 9.24 | 0 | 15.51 | 8.91 | 63.77 | >660 | >660 |
| RRB6_4 | 0 | 0 | 15.51 | 96.36 | 318.06 | >660 | >660 |

Results for Plasma cfDNA from Advanced Adenoma and Polyps:

| | Sample type | | | | | | | |
|---|---|---|---|---|---|---|---|---|
| | AA | AA | AA | AA | Polyps | Polyps | Polyps | Polyps |
| | Assay | | | | | | | |
| | 331YP | 367YP | 439YP | 499YP | 592YP | 487YP | ZDW96P | 808YP |
| RRB10 | 129.69 | 49.83 | 246.51 | 3.96 | 67.32 | 55.44 | 126.39 | 20.79 |
| RRB13 | 0.33 | 0 | 0 | 0 | 0 | 1.32 | 0 | 0 |
| RRB14 | 0 | 0 | 0 | 0 | 0 | 0.66 | 1.98 | 0 |
| RRB16 | 0 | 6.6 | 0 | 0 | 0 | 8.25 | 7.26 | 0 |
| RRB17_1 | 0 | 0 | 6.27 | 0 | 0 | 0 | 3.63 | 3.3 |
| RRB17_2 | 11.22 | 19.9 | 18.15 | 14.52 | 0 | 7.26 | 0 | 7.92 |
| RRB20 | 19.47 | 0.33 | 20.13 | 3.96 | 8.58 | 0 | 6.6 | 0 |
| RRB21_4 | 0 | 0 | 16.17 | 0 | 0 | 0 | 0 | 0 |
| RRB26-2 | 0 | 2.97 | 0 | 0 | 0 | 0 | 0.99 | 1.32 |
| RRB30 | 0 | 7.92 | 18.48 | 0 | 0 | 0 | 7.26 | 14.62 |
| RRB6_4 | 0 | 0 | 0 | 0 | 0 | 0 | 20.79 | 0 |

Results for cfDNA from Healthy Controls:

| Sample type | Healthy control | Healthy control | Healthy control | Healthy control | Healthy control |
|---|---|---|---|---|---|
| Assay | 1024YP | 1040YP | 1074YP | 814Y | 826Y |
| RRB10 | 40.92 | 35.31 | 138.93 | 95.91 | 71.76 |
| RRB13 | 0 | 0 | 4.95 | 0 | 3.03 |
| RRB14 | 9.57 | 0 | 0 | 7.24 | 4.8 |
| RRB16 | 0 | 0 | 8.25 | 17.54 | 40.66 |
| RRB17_1 | 0 | 8.58 | 9.24 | 1.74 | 15.39 |
| RRB17_2 | 0 | 21.12 | 3.63 | 45.95 | 39.05 |
| RRB20 | 26.07 | 20.79 | 11.55 | 30.1 | 108.63 |
| RRB21_4 | 0 | 0 | 7.26 | 0 | 9.42 |
| RRB26-2 | 0 | 0 | 0 | 0 | 0 |
| RRB30 | 7.59 | 0 | 12.87 | 25.82 | 21.73 |
| RRB6_4 | 0 | 0 | 0 | 0 | 7.29 |

These results demonstrated overall higher methylation levels (as represented by higher copy numbers in the tables above) in CRC samples than in the other sample types for the methylation markers, with even higher methylation in CRC samples at more advanced stage, demonstrating that the current method can be used to quantify DNA methylation markers in plasma DNA.

### Example 13

### V1 Assay for Blood Sample Detection

### 1. Methods

(1) A total of 300 individuals including patients with CRC, advanced adenoma, benign polyps, healthy controls and asymptomatic volunteers were recruited either at Wenzhou Medical University first affiliated hospital or through public recruitment during 2016 to 2019.
(2) Ten mL of venous blood were collected into EDTA blood collection tubes from each individual.
(3) Plasma samples were collected by a routine two-step centrifugation and stored in nuclease-free Eppendorf tubes at −80° C.
(4) cfDNA was extracted by the Apostle cfDNA extraction kit, with 1 μL of extracted cfDNA quantified by Thermo Fisher Qubit kit.
(5) Preparation of quality control samples: the positive control sample was prepared by mixing HCT15 cell line DNA and buffy coat DNA from a healthy individual at a 1:99 ratio. The negative control sample was buffy coat DNA from a healthy individual. The positive and negative control samples were at 10 ng/μL. Twenty ng of each control sample was used for each reaction as the reference for evaluating the validity of the experiments. Details on the quality control samples are provided in example 3.
(6) cfDNA samples (between 5 to 100 ng), 20 ng positive control sample and 20 ng negative control samples were subject to bisulfite conversion with EZ methylation Gold kit, and eluted in 21 μL DNase/RNase free water.
(7) Multiplex real-time quantitative PCR (V1 assay) was used to quantify the methylation signals for the bisulfite converted DNA, using the primers and probes, reaction system and conditions of the V1 mqMSP assay.
A. The V1 assay analyzes MBSF9, MBSF10, MBSF15, MBSR5, MBSR6, MBSR7, MBSR8, MBSR9, MBSR11, MBSR16, and ACTB, with sequences shown in the table in example 4.
B. The preparation of mixtures of primers and probes are shown in example 5.
C. mqMSP reaction system and condition are shown in example 5.
D. Interpretation of results: On the Bio-rad CFX96 qPCR platform, set FAM threshold at 100.17 and VIC threshold at 33. On the ABI7500 qPCR platform, set FAM threshold at 0.2 and VIC threshold at 0.09. In the table below: + represents Cq values≤45, − represents no amplification signal. ΔCq=VIC(average Cq)−FAM(average Cq).

The validity of the qPCR reactions was first confirmed by the positive and negative control samples:

When the qPCR results for the control samples satisfied the standards in the table below, and the test samples were analyzed in the same batch of qPCR reactions with the control samples, the results for the test samples were considered valid.

| Results for control samples | FAM Cq | VIC Cq | Notes |
|---|---|---|---|
| Valid positive control | FAM(+/+) | VIC(+/+) | ΔCq ≥ −1 |
| Valid negative control | FAM(−/−) or FAM(+/−) or FAM(+/+) | VIC(+/+) | ΔCq < −1 |

Interpretation of qPCR Results for Test Samples:

| | | | Interpretation |
|---|---|---|---|
| VIC | FAM | Algorithm | of results |
| VIC(−/−) | FAM(−/−) | Quality control failed | Not valid |
| | FAM(+/−) | Quality control failed | Not valid |
| | FAM(+/+) | Quality control passed | ΔCq ≥ −1, Positive |
| | | Set VIC(−) as VIC Cq = 45, | result |
| | | ΔCq = VIC(average Cq) − | ΔCq < −1, Negative |
| | | FAM(average Cq) | result |
| VIC(+/−) | FAM(−/−) | Quality control failed | Not valid |
| | FAM(+/−) | Quality control failed | Not valid |
| | FAM(+/+) | Quality control passed | ΔCq ≥ −1, Positive |
| | | Set VIC(−) as VIC Cq = 45, | result |
| | | ΔCq = VIC(average Cq) − | ΔCq < −1, Negative |
| | | FAM(average Cq) | result |
| VIC(+/+) | FAM(−/−) | Quality control passed | ΔCq ≥ −1, Positive |
| | | Set FAM(−) as FAM Cq = | result |
| | | 45, ΔCq = VIC(average | ΔCq < −1, Negative |
| | | Cq) − FAM(average Cq) | result |
| | FAM(+/−) | Quality control passed | ΔCq ≥ −1, Positive |
| | | Set FAM(−) as FAM Cq = | result |
| | | 45, ΔCq = VIC(average | ΔCq < −1, Negative |
| | | Cq) − FAM(average Cq) | result |
| | FAM(+/+) | Quality control passed | ΔCq ≥ −1, Positive |
| | | ΔCq = VIC(average Cq) − | result |
| | | FAM(average Cq) | ΔCq < −1, Negative |
| | | | result |

### 2. Results

The clinical characteristics and detection rates for the enrolled 300 individuals are summarized below:

| | | | | | | Detection |
|---|---|---|---|---|---|---|
| | | Gender | | Median age | Positive | rate |
| Group | Numbers | Male | Female | (range) | number | (%) |
| CRC | | | | | | |
| Total | 58 | 38 | 20 | 69 (47-90) | 50 | 86.2 |
| Stage I | 14 | 7 | 7 | 64 (47-84) | 9 | 64.3 |
| Stage II | 19 | 13 | 6 | 71 (49-88) | 16 | 84.2 |
| Stage III | 10 | 8 | 2 | 72 (60-90) | 10 | 100 |
| Stage IV | 15 | 10 | 5 | 69 (55-79) | 15 | 100 |
| Advanced Adenoma | 48 | 34 | 14 | 56 (40-76) | 11 | 23 |
| Benign Polyps | 30 | 20 | 10 | 56 (41-83) | 12 | 40 |
| Healthy Controls | 48 | 18 | 30 | 57 (42-74) | 8 | 16.7 |
| Asymptomatic volunteers | 116 | 45 | 71 | 53 (45-76) | 16 | 13.8 |
| Total | 300 | 155 | 145 | | | |

The current method has a sensitivity of 86.21% for CRC detection at the specificity of 83.33%, with detection rates of 64.3%, 84.2%, 100% and 100% for stage I to IV, respectively.

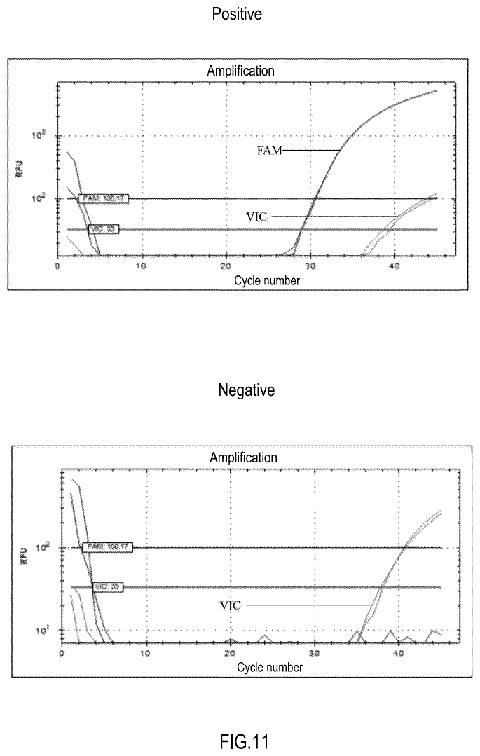

FIG. 11 shows a positive and a negative DNA methylation detection with plasma DNA.

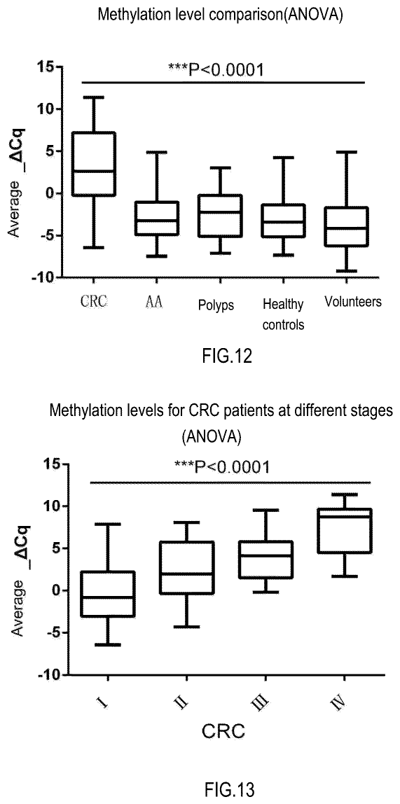

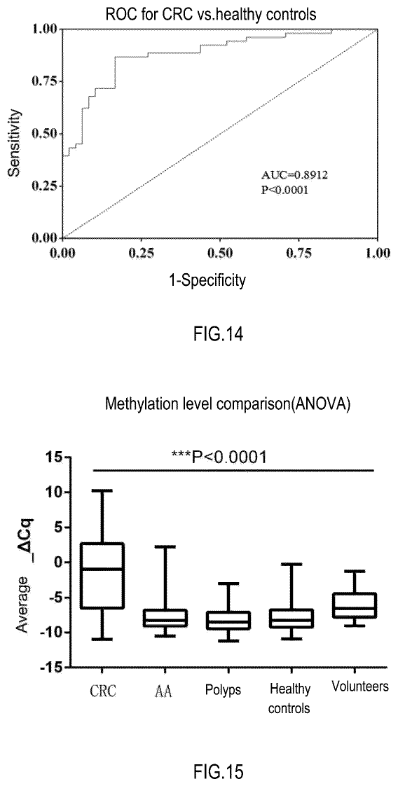

FIG. 12 shows plasma DNA methylation from CRC patients were significantly higher than other groups. FIG. 13 shows plasma DNA methylation levels correlated with tumor stage, with higher methylation levels in later stages. FIG. 14 shows the ROC curve analysis with an AUC value of 0.8912, demonstrating that the current method had good accuracy.

### Example 14

### V2 Assay for Blood Sample Detection

### 1. Methods

(1) A total of 305 individuals including patients with CRC, advanced adenoma, benign polyps, healthy controls and asymptomatic volunteers were recruited either at Wenzhou Medical University first affiliated hospital or through public recruitment during 2016 to 2019.
(2) Ten mL of venous blood were collected into EDTA blood collection tubes from each individual.
(3) Plasma samples were collected by a routine two-step centrifugation and stored in nuclease-free Eppendorf tubes at −80° C.
(4) cfDNA was extracted by the Apostle cfDNA extraction kit, with 1 μL of extracted cfDNA quantified by Thermo Fisher Qubit kit.
(5) Preparation of quality control samples: the positive control sample was prepared by mixing HCT15 cell line DNA and buffy coat DNA from a healthy individual at a 1:99 ratio. The negative control sample was buffy coat DNA from a healthy individual. The positive and negative control samples were at 10 ng/μL. Twenty ng of each control sample was used for each reaction as the reference for evaluating the validity of the experiments. Details on the quality control samples are provided in example 3.
(6) Two hundred nanograms of carrier DNA was added to each cfDNA samples (between 5 to 100 ng), positive control and negative control samples, followed by bisulfite conversion with EZ methylation Gold kit, and eluted in 21 μL DNase/RNase free water.
(7) Multiplex real-time quantitative PCR (V2 assay) was used to quantify the methylation signals for the bisulfite converted DNA, using the primers and probes, reaction system and conditions of the V2 mqMSP assay.
A. The V2 assay analyzes MBSF9, MBSF8, MBSR13, MBSR16, NDRG4, QKI and ACTB, with sequences shown in the table in example 4.
B. The preparation of mixtures of primers and probes are shown in example 7.
C. mqMSP reaction system and condition are shown in example 7.
D. Interpretation of results: On the Bio-rad CFX96 qPCR platform, set FAM threshold at 100.17 and VIC threshold at 33. On the ABI7500 qPCR platform, set FAM threshold at 0.2 and VIC threshold at 0.09. In the table below: + represents Cq values≤45, − represents no amplification signal. ΔCq=VIC(average Cq)−FAM(average Cq).

The validity of the qPCR reactions was first confirmed by the positive and negative control samples:

When the qPCR results for the control samples satisfied the standards in the table below, and the test samples were analyzed in the same batch of qPCR reactions with the control samples, the results for the test samples were considered valid.

| Results for control samples | FAM Cq | VIC Cq |
|---|---|---|
| Valid positive control | FAM(+/+) | VIC(+/+) |
| Valid negative control | FAM(−/−) | VIC(+/+) |

Interpretation of qPCR Results for Test Samples:

| VIC | FAM | Algorithm | Interpretation of results |
|---|---|---|---|
| VIC(−/−) | FAM(−/−) | Quality control failed | Not valid |
| | FAM(+/−) | Quality control failed | Not valid |
| | FAM(+/+) | Quality control passed | Positive result |
| VIC(+/−) | FAM(−/−) | Quality control failed | Not valid |
| | FAM(+/−) | Quality control failed | Not valid |
| | FAM(+/+) | Quality control passed | Positive result |
| VIC(+/+) | FAM(−/−) | Quality control passed | Negative result |
| | FAM(+/−) | Quality control passed | Positive result |
| | FAM(+/+) | Quality control passed | Positive result |

### 2. Results

The clinical characteristics and detection rates for the enrolled 305 individuals are summarized below:

| | | | | | | Detection |
|---|---|---|---|---|---|---|
| | | Gender | | Median age | Positive | rate |
| Group | Numbers | Male | Female | (range) | number | (%) |
| CRC | | | | | | |
| Total | 114 | 74 | 40 | 64 (45-75) | 77 | 67.5 |
| Stage | 31 | 17 | 14 | 64 (53-75) | 13 | 42 |
| Stage II | 28 | 20 | 8 | 69 (45-75) | 21 | 75 |
| Stage III | 31 | 21 | 10 | 61 (47-75) | 21 | 67.7 |
| Stage IV | 24 | 16 | 8 | 62 (45-72) | 22 | 91.7 |
| Advanced Adenoma | 47 | 36 | 11 | 58 (46-75) | 3 | 6.4 |
| Benign Polyps | 45 | 31 | 14 | 59 (49-75) | 5 | 11.1 |
| Healthy Controls | 57 | 21 | 36 | 54 (46-74) | 1 | 1.8 |
| Volunteers | 42 | 16 | 26 | 54 (45-66) | 3 | 7.1 |
| Total | 305 | 178 | 127 | | | |

The current method has a sensitivity of 67.54% for CRC detection at the specificity of 98.25%, with detection rates of 42%, 75%, 67.7% and 91.7% for stage I to IV, respectively.

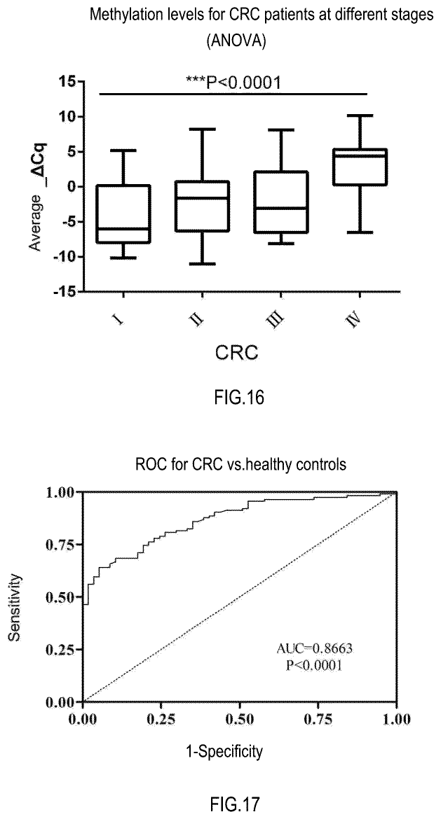

FIG. 15 shows plasma DNA methylation from CRC patients were significantly higher than other groups. FIG. 16 shows plasma DNA methylation levels correlated with tumor stage, with higher methylation levels in later stages. FIG. 17 shows the ROC curve analysis with an AUC value of 0.8663, demonstrating that the current method had good accuracy.

### Example 15

### V4 Assay for Blood Sample Detection

### 1. Methods

(1) A total of 194 individuals including patients with CRC, advanced adenoma, benign polyps, gastroenteritis, esophageal carcinoma, lung cancer and healthy controls were recruited either at Wenzhou Medical University first affiliated hospital or through public recruitment during 2016 to 2019.
(2) Ten mL of venous blood were collected into EDTA blood collection tubes from each individual.
(3) Plasma samples were collected by a routine two-step centrifugation and stored in nuclease-free Eppendorf tubes at −80° C. The samples were randomly re-labeled by a third party for blinded testing. The person who performed the V4 assay had no knowledge of sample types and performed the following steps.
(4) cfDNA was extracted by the Apostle cfDNA extraction kit, with 1 μL of extracted cfDNA quantified by Thermo Fisher Qubit kit.
(5) Preparation of quality control samples: the positive control sample was prepared by mixing HCT15 cell line DNA and buffy coat DNA from a healthy individual at a 1:99 ratio. The negative control sample was buffy coat DNA from a healthy individual. The positive and negative control samples were at 10 ng/μL. Twenty ng of each control sample was used for each reaction as the reference for evaluating the validity of the experiments. Details on the quality control samples are provided in example 3.
(6) Two hundred nanograms of carrier DNA was added to each cfDNA samples (between 5 to 100 ng), positive control and negative control samples, followed by bisulfite conversion with EZ methylation Gold kit, and eluted in 21 μL DNase/RNase free water.
(7) Multiplex real-time quantitative PCR (V4 assay) was used to quantify the methylation signals for the bisulfite converted DNA, using the primers and probes, reaction system and conditions of the V4 mqMSP assay.
A. The V4 assay analyzes MBSF9, MBSF8, MBSR13, QKI, RD1 and RD2 (without the RD2_R primer) and ACTB, with sequences shown in the table in example 4.
B. The preparation of mixtures of primers and probes are shown in example 9.
C. mqMSP reaction system and condition are shown in example 9.
D. Interpretation of results: On the Bio-rad CFX96 qPCR platform, set FAM threshold at 100.17 and VIC threshold at 33. On the ABI7500 qPCR platform, set FAM threshold at 0.2 and VIC threshold at 0.09. In the table below: + represents Cq values≤45, − represents no amplification signal. ΔCq=VIC(average Cq)−FAM(average Cq).

The validity of the qPCR reactions was first confirmed by the positive and negative control samples:

When the qPCR results for the control samples satisfied the standards in the table below, and the test samples were analyzed in the same batch of qPCR reactions with the control samples, the results for the test samples were considered valid.

| Results for control samples | FAM Cq | VIC Cq |
|---|---|---|
| Valid positive control | FAM(+/+) | VIC(+/+) |
| Valid negative control | FAM(−/−) | VIC(+/+) |

Interpretation of qPCR Results for Test Samples:

| VIC | FAM | Algorithm | Interpretation of results |
|---|---|---|---|
| VIC(−/− ) | FAM(−/−) | Quality control failed | Not valid |
| | FAM(+/−) | Quality control failed | Not valid |
| | FAM(+/+) | Quality control passed | Positive result |
| VIC(+/−) | FAM(−/−) | Quality control failed | Not valid |
| | FAM(+/−) | Quality control failed | Not valid |
| | FAM(+/+) | Quality control passed | Positive result |
| VIC(+/+) | FAM(−/−) | If VIC(average Cq) > 40, | Not valid or Negative |
| | | Quality control failed; | result |
| | | If VIC(average Cq ≤ 40, | |
| | | Quality control passed | |
| | FAM(+/−) | Quality control passed | Positive result |
| | FAM(+/+) | Quality control passed | Positive result |

### 2. Results

The clinical characteristics and detection rates for the enrolled 194 individuals are summarized below:

| | | | | | | Detection |
|---|---|---|---|---|---|---|
| | | Gender | | Median age | Positive | rate |
| Group | Numbers | Male | Female | (range) | number | (%) |
| CRC | | | | | | |
| Total | 137 | 95 | 42 | | 110 | 80.3 |
| Stage I | 43 | 30 | 13 | 64 (46-75) | 32 | 74.4 |
| Stage II | 54 | 35 | 19 | 66 (45-75) | 40 | 74.1 |
| Stage III | 20 | 15 | 5 | 66 (55-75) | 19 | 95 |
| Stage IV | 20 | 15 | 5 | 62 (45-71) | 19 | 95 |
| Advanced Adenoma | 11 | 6 | 5 | 60 (45-67) | 2 | 18.2 |
| Benign Polyps | 11 | 7 | 4 | 55 (47-67) | 5 | 45.5 |
| Gastroenteritis | 12 | 5 | 7 | 55 (45-61) | 2 | 16.7 |
| Esophageal carcinoma | 6 | 5 | 1 | 64 (50-72) | 6 | 100 |
| Lung cancer | 2 | 1 | 1 | 71 (65-76) | 1 | 50 |
| Healthy Controls | 15 | 8 | 7 | 51 (45-63) | 3 | 20 |
| Total | 194 | 127 | 67 | 63 (45-76) | | |

The current method has a sensitivity of 80.3% for CRC detection at the specificity of 80%, with detection rates of 74.4%, 74.1%, 95% and 95% for stage I to IV, respectively.

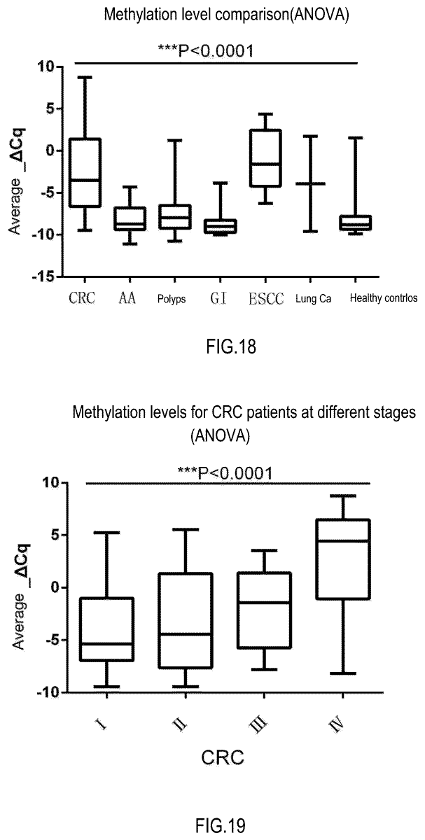

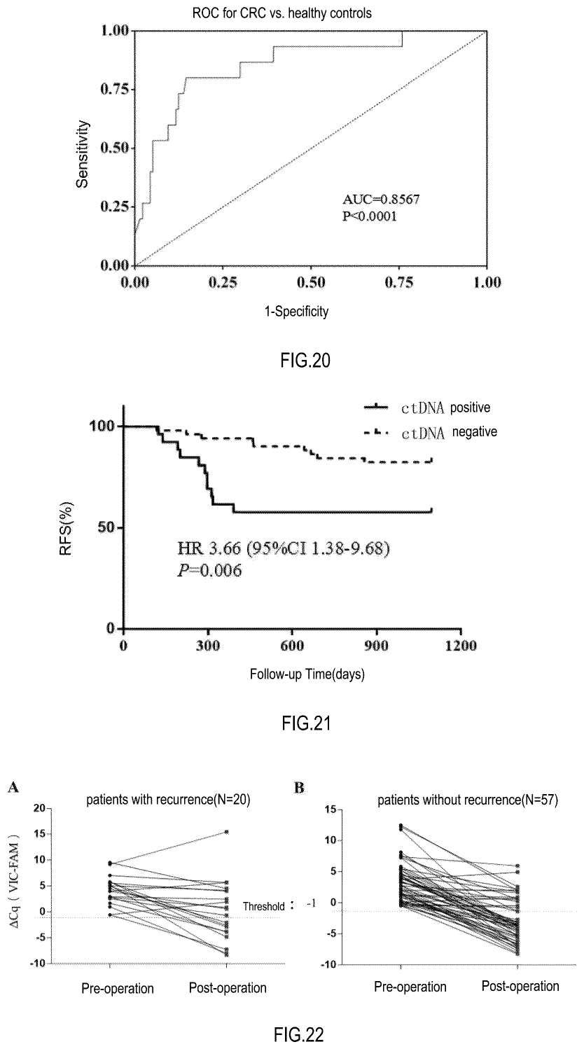

FIG. 18 shows plasma DNA methylation from CRC patients were significantly higher than other groups. FIG. 19 shows plasma DNA methylation levels correlated with tumor stage, with higher methylation levels in later stages. FIG. 20 shows the ROC curve analysis with an AUC value of 0.8567, demonstrating that the current method had good accuracy.

### Example 16

### DNA Methylation Markers for CRC Prognosis and Recurrence Prediction

### 1. Methods

(1) A total of 86 CRC patients with CRC receiving surgical resection at Wenzhou Medical University first affiliated hospital were enrolled during 2016 to 2019. Blood samples were collected before and after surgery and at subsequent follow-ups.
(2) Blood processing and quality control sample preparation were the same as in example 13.
(3) Multiplex real-time quantitative PCR (V1 assay) was used to quantify the methylation signals for the bisulfite converted DNA, using the primers and probes, reaction system and conditions of the V1 mqMSP assay, as described in example 13.

### 2. Results

Out of the 86 CRC patients, 77 were positive for pre-operative ctDNA (positive detection rate: 89.5%) while 9 were negative. The 77 patients with positive pre-operative ctDNA were further tested using the post-operative and follow-up blood ctDNA to evaluate prognosis and recurrence, together with patient clinical information and other monitoring markers.

Out of the 77 patients, 20 patients relapsed while others did not during follow-up. Out of the 20 recurred patients, 11 were positive for post-operative ctDNA while 9 were negative. For the 51 non-recurred patients, 15 were positive for post-operative ctDNA while 36 were negative. Survival analysis (FIG. 21) based on post-operative ctDNA positivity and patient relapse showed a significantly shorter (P=0.006) recurrence free survival (RFS) for patients with positive post-operative ctDNA. For the patients with positive pre-operative ctDNA, ctDNA methylation levels dropped to a variable extent after surgery in vast majority of the patients (FIG. 22).

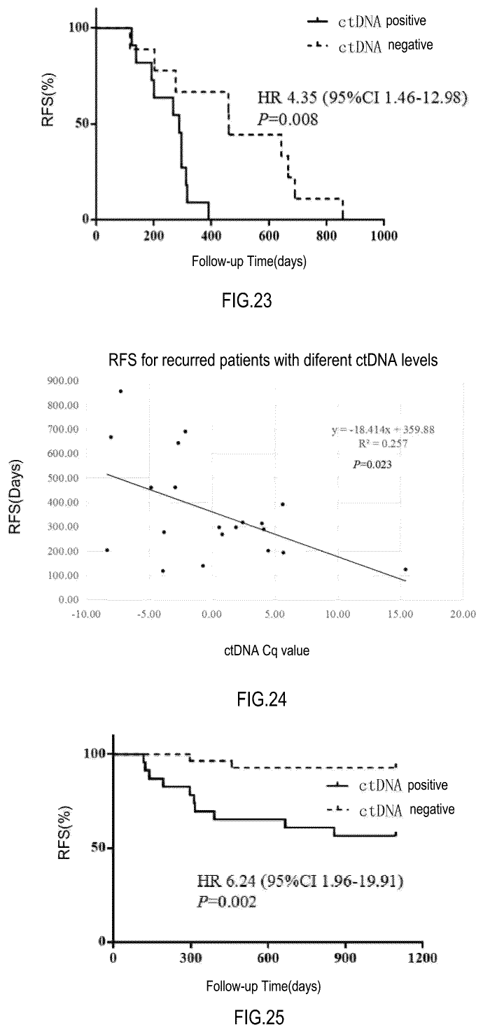

Additionally, for the 20 patients with recurrence, RFS was significantly shorter for post-operative ctDNA positive patients than negative patients (median RFS 288 vs 460 days, P=0.008, FIG. 23), demonstrating that patients with positive post-operative ctDNA recurred earlier. Quantitative analysis of patients with positive post-operative ctDNA showed that ctDNA methylation level was negatively correlated with RFS, with higher levels of ctDNA methylation suggesting poorer prognosis (FIG. 24). These data demonstrated that post-operative ctDNA detection can be used to evaluate patient prognosis after surgery.

Out of the 77 patients, 51 patients had at least one follow-up blood sample collected one month or later after surgery. We used these follow-up blood samples for ctDNA detection to further evaluate patient recurrence. Among the 4 recurred patients with negative post-operative ctDNA, three patients were positive for ctDNA collected at follow-up or at relapse while the other one patient remained negative for follow-up ctDNA. For the non-recurred patients with positive post-operative ctDNA, five patients were negative for follow-up blood. RFS survival curve analysis for these 51 patients with follow-up blood ctDNA detection revealed that patients with positive ctDNA had significantly shorter RFS (P=0.002) (FIG. 25), demonstrating that ctDNA detection during follow-up can effectively assess patient recurrence, with positive detection pointing to disease relapse and metastasis risk.

### Example 17

DNA methylation markers for dynamic monitoring of the entire life cycle of CRC management and treatment including assessing neoadjuvant treatment efficacy, post-surgery evaluation, and post-surgery monitoring.

### 1. Methods

(1) A rectal cancer patient who received neoadjuvant therapy and surgery at Wenzhou Medical University first affiliated hospital was enrolled for collection of 12 blood samples at various time points including prior to neoadjuvant therapy, during neoadjuvant therapy, prior to surgery, post-surgery and during dynamic follow-up time points.
(2) Blood processing and quality control sample preparation were the same as in example 13.
(3) Multiplex real-time quantitative PCR (V1 assay) was used to quantify the methylation signals for the bisulfite converted DNA, using the primers and probes, reaction system and conditions of the V1 mqMSP assay, as described in example 13.

### 2. Results

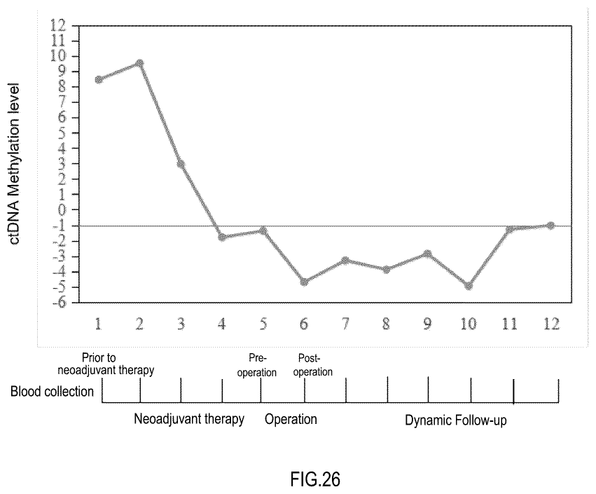

FIG. 26 shows the dynamic changes of ctDNA levels for the patient during treatments and follow-ups. The patient had a positive ctDNA detection prior to neoadjuvant therapy. CtDNA detection turned negative after the patient finished the neoadjuvant therapy, and remained negative pre-operation, post-operation and during multiple follow-up time points. Imaging analysis showed no evidence of recurrence. The patient had good prognosis. These results demonstrated the clinical utility of the current method for assessing neoadjuvant treatment efficacy, post-surgery evaluation, and post-surgery monitoring.

### References

[1] Bray F, Ferlay J, Soerjomataram I, et al. Global cancer statistics 2018: GLOBOCAN estimates of incidence and mortality worldwide for 36 cancers in 185 countries[J]. CA: A Cancer Journal for Clinicians, 2018, 68(6).
[2] Chen W, Zheng R, Baade P D, et al. Cancer statistics in China, 2015[J]. CA: A Cancer Journal for Clinicians, 2016, 66(2):115-132.
[3] Siegel R L, Miller K D, Fedewa S A, et al. Colorectal cancer statistics, 2017[J]. CA: A Cancer Journal for Clinicians, 2017, 67(3): 177-193.
[4] Carethers J M, Jung B H. Genetics and Genetic Biomarkers in Sporadic Colorectal Cancer[J]. Gastroenterology, 2015, 149(5): 1177-1190.e3.
[5] Guinney J, Dienstmann R, Wang X, et al. The consensus molecular subtypes of colorectal cancer[J]. Nature medicine, 2015.
[6] Phipps A I, Limburg P J, Baron J A, et al. Association Between Molecular Subtypes of Colorectal Cancer and Patient Survival[J]. Gastroenterology, 2015, 148(1):77-87.e2.
[7] Portela A, Esteller M. Epigenetic modifications and human disease[J]. Nature Biotechnology, 2010, 28(10): 1057-1068.
[8] Lao V V, Grady W M. Epigenetics and colorectal cancer[J]. Nature Reviews Gastroenterology & Hepatology, 2011, 8(12): 686-700.
[9] Choi Y, Sateia H F. Peairs K S, et al. Screening for colorectal cancer[J]. Seminars in Oncology, 2017, 44(1): 34-44.
[10] Sargent D, Sobrero A, Grothey A, et al. Evidence for Cure by Adjuvant Therapy in Colon Cancer: Observations Based on Individual Patient Data From 20,898 Patients on 18 Randomized Trials[J]. Journal of Clinical Oncology, 2009, 27(6): 872-877.
[11] Pahlman L A, Hohenberger W M, Matzel K, et al. Should the Benefit of Adjuvant Chemotherapy in Colon Cancer Be Re-Evaluated?[J]. Journal of Clinical Oncology, 2016:JCO.2015.65.3048.
[12] BÖCkelman C, Engelmann B E, Kaprio T, et al. Risk of recurrence in patients with colon cancer stage II and III: A systematic review and meta-analysis of recent literature[J]. Acta Oncologica, 2015, 54(1):5-16.
[13] Snyder R A, Hu C-Y. Cuddy A, et al. Association Between Intensity of Posttreatment Surveillance Testing and Detection of Recurrence in Patients With Colorectal Cancer[J]. Jama, 2018, 319(20): 2104.
[14] Elferink M a G, De Jong K P, Klaase J M, et al. Metachronous metastases from colorectal cancer: a population-based study in North-East Netherlands[J]. International Journal of Colorectal Disease, 2014, 30(2): 205-212.
[15] colon_NCCN GUIDELINES FOR TREATMENT OF CANCER BY SITE.2018.
[16] Chao M, Gibbs P. Caution Is Required Before Recommending Routine Carcinoembryonic Antigen and Imaging Follow-Up for Patients With Early-Stage Colon Cancer[J]. Journal of Clinical Oncology, 2009, 27(36): e279-e280.
[17] rectal_NCCN GUIDELINES FOR TREATMENT OF CANCER BY SITE.2018.
[18] Wang J, Chang S, Li G, et al. Application of liquid biopsy in precision medicine: opportunities and challenges[J]. Frontiers of Medicine, 2017, 11(4).
[19] Ellen, Heitzer, Imran, et al. Current and future perspectives of liquid biopsies in genomics-driven oncology.[J]. Nature reviews. Genetics, 2018.
[20] Normanno N, Cervantes A, Ciardiello F, et al. The liquid biopsy in the management of colorectal cancer patients: Current applications and future scenarios. Cancer Treat Rev. 2018; 70:1-8.
[21] Wan J C M, Massie C, Garcia-Corbacho J, et al. Liquid biopsies come of age: towards implementation of circulating tumour DNA[J]. Nature Reviews Cancer, 2017, 17(4):223-238.
[22] Cottrell S E, Distler J, Goodman N S, et al. A real-time PCR assay for DNA-methylation using methylation-specific blockers. Nucleic Acids Res. 2004; 32(1):e10
[23] Epi proColon instructions.pdf.
[24] Church T R, Wandell M, Lofton-Day C, et al. Prospective evaluation of methylatedSEPT9in plasma for detection of asymptomatic colorectal cancer[J]. Gut, 2014, 63(2): 317-325.
[25] Sun J, Fei F, Zhang M, et al. The role of mSEPT9 in screening, diagnosis, and recurrence monitoring of colorectal cancer[J]. BMC Cancer, 2019, 19(1).
[26] Lee H S, Hwang S M, Kim T S, et al. Circulating Methylated Septin 9 Nucleic Acid in the Plasma of Patients with Gastrointestinal Cancer in the Stomach and Colon[J]. Translational Oncology, 2013, 6(3):290-IN4.
[27] Guo S, Diep D, Plongthongkum N, et al. Identification of methylation haplotype blocks aids in deconvolution of heterogeneous tissue samples and tumor tissue-of-origin mapping from plasma DNA[J]. Nature Genetics, 2017, 49(4):635-642.
[28] Guo H, Zhu P, Wu X, et al. Single-cell methylome landscapes of mouse embryonic stem cells and early embryos analyzed using reduced representation bisulfite sequencing[J]. Genome Research, 2013, 23(12):2126-2135.
[29] Liu M C, Oxnard G R, Klein E A, et al. Sensitive and specific multi-cancer detection and localization using methylation signatures in cell-free DNA[J]. Annals of Oncology, 2020.
[30] Imperiale T F, Ransohoff D F, Itzkowitz S H, et al. Multitarget Stool DNA Testing for Colorectal-Cancer Screening[J]. New England Journal of Medicine, 2014, 370(14): 1287-1297.
[31] Rasmussen S L, Krarup H B, Sunesen K G, et al. Hypermethylated DNA as a biomarker for colorectal cancer: a systematic review[J]. Colorectal Disease, 2016, 18(6): 549-561.
[32] Lofton-Day C, Model F, Devos T, et al. DNA Methylation Biomarkers for Blood-Based Colorectal Cancer Screening[J]. Clinical Chemistry, 2008, 54(2): 414-423.
[33] Wasserkort R, Kalmar A, Valcz G, et al. Aberrant septin 9 DNA methylation in colorectal cancer is restricted to a single, CpG island.[J]. BMC Cancer, 2013, 13(1):1-11.

1. A method for diagnosis of presence of colorectal cancer in a subject, prognosis of a colorectal cancer patient after surgery, predicting recurrence for a colorectal cancer patient after surgery, and assessing treatment efficacy for a colorectal cancer patient, comprising determining methylation level of cell-free DNA by detecting methylation marker(s) in the said cell-free DNA, wherein when the methylation level in the test subject is higher than the levels in control samples, the subject may suffer from colorectal cancer, the subject with colorectal cancer may have poor prognosis after surgery, may be more likely to recur, or may have poor treatment efficacy, the said methylation marker(s) may be one or more markers selected from Table 2, Table 3 and Table 8.

2. The method of claim 1, wherein the detection of DNA methylation markers is performed by multiplex quantitative methylation specific PCR, with methylation markers selected from any of the following:
1) one or more markers selected from MBSF9, MBSF10, MBSF15, MBSR5, MBSR6, MBSR7, MBSR8, MBSR9, MBSR11 and MBSR16;
2) one or more markers selected from MBSF9, MBSF8, MBSR13, MBSR16, NDRG4 and QKI;
3) one or more markers selected from MBSF9, MBSF8, MBSR13, NDRG4, QKI, RD1 and RD2;
4) one or more markers selected from MBSF9, MBSF8, MBSR13, QKI, RD1 and RD2,
optionally, the said multiplex quantitative methylation specific PCR comprises the detection of ACTB as an internal control.

3. The method of claim 2, wherein the said multiplex quantitative methylation specific PCR uses primers and probes for the DNA methylation markers in claim 2 and primers and probe for the internal control ACTB, wherein the said primers and probes comprise the sequences listed in Table 4, or sequences with at least 80% identity to those listed in Table 4.

4. The method of claim 3, wherein when the DNA methylation marker(s) are one or more markers selected from MBSF9, MBSF8, MBSR13, QKI, RD1 and RD2, with the RD2_F primer not used in multiplex quantitative methylation specific PCR.

5. The method of claim 1, wherein the detection of DNA methylation markers is by multiplex quantitative methylation specific PCR with DNA methylation markers dividing into two or more groups, with probes labeled with different fluorescence for each group and the internal control.

6. The method of claim 5, wherein the methylation markers are divided into two groups, wherein Group 1 comprises MBSF9, MBSR16, MBSF8, MBSR13, NDRG4, NPY and QKI, with primers and probes comprising the sequences listed in Table 5, or sequences with at least 80% identity to those listed in Table 5, and Group 2 comprises MBSF15, MBSR5, MBSR6, MBSR7, MBSR8 and MBSR9, with primers and probes comprising the sequences listed in Table 6, or sequences with at least 80% identity to those listed in Table 6, and additionally, ACTB is used as the internal control, with primers and probe comprising the sequences listed in Table 7, or sequences with at least 80% identity to those listed in Table 7.

7. The method of claim 1, wherein MALDI-TOF mass spectrometry is used for detecting one or more methylation markers selected from RRB10, RRB13, RRB14, RRB16, RRB17_1, RRB17_2, RRB20, RRB21_4, RRB26_2, RRB2, RRB30, RRB6_1, RRB6_4 and RRB6_5, optionally, the said MALDI-TOF mass spectrometry method also detects the internal control ACTB gene.

8. The method of claim 7, wherein the methylation markers and the internal control gene are co-amplified with their respective competitors at known copy numbers, using PCR primers and extension primers, wherein the copy numbers of the DNA methylation markers are calculated based on the signal ratios between the methylation markers and their respective competitors,
wherein the PCR primers for the methylation markers and the internal control gene comprise the sequences listed in Table 9, or sequences with at least 80% identity to those listed in Table 9, wherein the extension primers for the methylation markers and the internal control gene comprise the sequences listed in Table 10, or sequences with at least 80% identity to those listed in Table 10, and wherein the competitors for the methylation markers and the internal control gene comprise the sequences listed in Table 11, or sequences with at least 80% identity to those listed in Table 11.

9. The method of claim 1, wherein the said sample is selected from body fluids, blood, serum, plasma, urine, saliva, sweat, sputum, semen, mucus, tear, lymphatic fluid, amniotic fluid, interstitial fluid, pulmonary lavage fluid, cerebrospinal fluid, stool and tissues.

10. DNA methylation marker(s) for diagnosis of presence of colorectal cancer in a subject, prognosis of a colorectal cancer patient after surgery, predicting recurrence for a colorectal cancer patient after surgery, and assessing treatment efficacy for a colorectal cancer patient, wherein the said methylation marker(s) are one or more markers selected from Table 2, Table 3 and Table 8.

11. DNA methylation markers of claim 10, selected from the group comprising MBSF9, MBSF10, MBSF15, MBSR5, MBSR6, MBSR7, MBSR8, MBSR9, MBSR11, MBSR16, MBSF8, MBSR13, RD1, RD2, NPY, NDRG4, QKI, RRB10, RRB13, RRB14, RRB16, RRB17_1, RRB17_2, RRB20, RRB21_4, RRB26_2, RRB2, RRB30, RRB6_1, RRB6_4 and RRB6_5.

12. A diagnostic kit for diagnosis of presence of colorectal cancer in a subject, prognosis of a colorectal cancer patient after surgery, predicting recurrence for a colorectal cancer patient after surgery, and assessing treatment efficacy for a colorectal cancer patient, comprising reagents for detecting DNA methylation markers, wherein the said marker(s) are one or more markers selected from Table 2, Table 3 and Table 8.

13. A diagnostic kit of claim 12, wherein the said methylation marker(s) are one or more markers selected from the group comprising MBSF9, MBSF10, MBSF15, MBSR5, MBSR6, MBSR7, MBSR8, MBSR9, MBSR11, MBSR16, MBSF8, MBSR13, RD1, RD2, NPY, NDRG4 and QKI, optionally, the kit also contains reagents for detecting the internal control ACTB.

14. A diagnostic kit of claim 13, wherein the reagents for detecting the methylation markers and the internal control gene comprise the sequences listed in Table 4.

15. A diagnostic kit of claim 12, wherein the said methylation marker(s) are one or more markers selected from the group comprising RRB10, RRB13, RRB14, RRB16, RRB17_1, RRB17_2, RRB20, RRB21_4, RRB26_2, RRB2, RRB30, RRB6_1, RRB6_4 and RRB6_5, optionally, the kit also contains reagents for detecting the internal control ACTB.

16. A diagnostic kit of claim 15, wherein the reagents for detecting the methylation markers and the internal control gene comprise the sequences listed in Table 9, Table 10 and Table 11, or sequences with at least 80% identity to those listed in Table 9, Table 10 and Table 11.

17. A diagnostic kit of claim 13, wherein the said methylation marker(s) are selected from any of the following:
1) one or more markers selected from the group comprising MBSF9, MBSF10, MBSF15, MBSR5, MBSR6, MBSR7, MBSR8, MBSR9, MBSR11 and MBSR16;
2) one or more markers selected from the group comprising MBSF9, MBSF8, MBSR13, MBSR16, NDRG4 and QKI;
3) one or more markers selected from the group comprising MBSF9, MBSF8, MBSR13, NDRG4, QKI, RD1 and RD2;
4) one or more markers selected from the group comprising MBSF9, MBSF8, MBSR13, QKI, RD1 and RD2.

18. Polynucleotides with sequences with at least 80% identity to those selected from SEQ ID NOs:1, 2 and 9-120.
